(19) United States
(12) Patent Application Publication
(10) Pub. No.: US 2008/0214455 A1
(43) Pub. Date: Sep. 4, 2008

(54) Novel Chemical Compounds

(75) Inventors: Kevin Duffy, Collegeville, PA (US); Deping Chai, Collegeville, PA (US); Mirela Colon, Collegeville, PA (US); Duke M. Fitch, Collegeville, PA (US); Sarah Rae King, Rural Retreat, VA (US); Antony N. Shaw, Collegeville, PA (US); Rosanna Tedesco, Collegeville, PA (US); Kenneth Wiggall, Collegeville, PA (US); Michael N. Zimmerman, Collegeville, PA (US); Neil W. Johnson, Collegeville, PA (US); Jiri Kasparec, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 11/912,969

(22) PCT Filed: May 3, 2006
(86) PCT No.: PCT/US2006/017142

§ 371 Date: Apr. 3, 2008

Related U.S. Application Data
(60) Provisional application No. 60/677,284, filed on May 3, 2005.

(51) Int. Cl. C07D 417/02; C07D 417/14; A61K 31/5377; A61K 31/498; A61K 31/496; A61K 31/4709; A61K 38/22; A61P 7/00

(52) U.S. Cl. 514/12; 544/116; 514/234.8; 514/249; 544/353; 546/152; 514/314; 544/363; 514/253.06; 514/235.2; 544/128

(57) ABSTRACT

This invention relates to newly identified compounds for inhibiting hYAK3 proteins and methods for treating diseases associated with hYAK3 activity.

### FIELD OF THE INVENTION

This invention relates to newly identified compounds for inhibiting hYAK3 proteins and methods for treating diseases associated with hYAK3 activity.

### BACKGROUND OF THE INVENTION

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein serine/threonine kinases (PSTK) and phosphatases. Serine/threonine phosphorylation is a major mediator of signal transduction in multicellular organisms. Receptor-bound, membrane-bound and intracellular PSTKs regulate cell proliferation, cell differentiation and signalling processes in many cell types.

Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are potential targets for drug design.

A subset of PSTKs are involved in regulation of cell cycling. These are the cyclin-dependent kinases or CDKs (Peter and Herskowitz, Cell 1994: 79,181-184). CDKs are activated by binding to regulatory proteins called cyclins and control passage of the cell through specific cell cycle checkpoints. For example, CDK2 complexed with cyclin E allows cells to progress through the G1 to S phase transition. The complexes of CDKs and cyclins are subject to inhibition by low molecular weight proteins such as p16 (Serrano et al, Nature 1993: 366, 704), which binds to and inhibits CDK4. Deletions or mutations in p16 have been implicated in a variety of tumors (Kamb et al, Science 1994: 264, 436-440). Therefore, the proliferative state of cells and diseases associated with this state are dependent on the activity of CDKs and their associated regulatory molecules. In diseases such as cancer where inhibition of proliferation is desired, compounds that inhibit CDKs may be useful therapeutic agents. Conversely, activators of CDKs may be useful where enhancement of proliferation is needed, such as in the treatment of immunodeficiency.

YAK1, a PSTK with sequence homology to CDKs, was originally identified in yeast as a mediator of cell cycle arrest caused by inactivation of the cAMP-dependent protein kinase PKA (Garrett et al, Mol Cell Biol. 1991: 11-6045-4052). YAK1 kinase activity is low in cycling yeast but increases dramatically when the cells are arrested prior to the S-G2 transition. Increased expression of YAK1 causes growth arrest in yeast cells deficient in PKA. Therefore, YAK1 can act as a cell cycle suppressor in yeast.

Our U.S. Pat. No. 6,323,318 describes two novel human homologs of yeast YAK1 termed hYAK3-2, one protein longer than the other by 20 amino acids. hYAK3-2 proteins (otherwise reported as REDK-L and REDK-S in Blood, 1 May 2000, Vol 95, No. 9, pp2838) are primarily localized in the nucleus. hYAK-2 proteins (hereinafter simply referred as hYAK3 or hYAK3 proteins) are present in hematopoietic tissues, such as bone marrow and fetal liver, but the RNA is expressed at significant levels only in erythroid or erthropoietin (EPO)-responsive cells. Two forms of REDK cDNAs appear to be alternative splice products. Antisense REDK oligonucleotides promote erythroid colony formation by human bone marrow cells, without affecting colony-forming unit (CFU)-GM, CFU-G, or CFU-GEMM numbers. Maximal numbers of CFU-E and burst-forming unit-erythroid were increased, and CFU-E displayed increased sensitivity to suboptimal EPO concentrations. The data indicate that REDK acts as a brake to retard erythropoiesis. Thus inhibitors of hYAK3 proteins are expected to stimulate proliferation of cells in which it is expressed. More particularly, inhibitors of hYAK3 proteins are useful to treat or prevent diseases of the erythroid and hematopoietic systems associated with hYAK3 activity, including but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia.

### SUMMARY OF THE INVENTION

This invention relates to novel compounds of Formula (I):

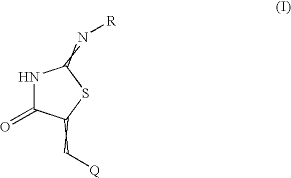

in which

R is selected form: aryl and substituted aryl; and

### Q is

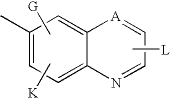

wherein

A is selected from CR⁵⁰ and N,
where R⁵⁰, G, K and L are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, —C(O)OR¹⁰, —C(O)NR¹¹R¹², oxo and cyano,
   where, R¹⁰ is selected form hydrogen, C₁-C₄alkyl, aryl and
   trifluoromethyl, and R¹¹ and R¹² are independently selected form hydrogen, C₁-C₄alkyl, aryl and trifluoromethyl,
   provided that at least one of G, K, L and R⁵⁰, when R⁵⁰ is present, is not hydrogen;
      and/or pharmaceutically acceptable salts, hydrates, solvates and pro-drugs thereof.

This invention relates a method of inhibiting hYAK3 in a mammal; comprising, administering to the mammal a therapeutically effective amount of a compound of the formula (I).

This invention relates to a method of treating or preventing diseases of the erythroid and hematopoietic systems, caused by the hYAK3 imbalance or inappropriate activity including, but not limited to, anemias due to renal insufficiency or to chronic disease, such as autoimmunity, HIV, or cancer, and drug-induced anemias, myelodysplastic syndrome, aplastic anemia and myelosuppression, and cytopenia; comprising administering to a mammal a therapeutically effective amount of a compound of formula (I).

In a further aspect of the invention there is provided novel processes and novel intermediates useful in preparing the presently invented hYAK3 inhibiting compounds.

Included in the present invention are pharmaceutical compositions that comprise a pharmaceutical carrier and compounds useful in the methods of the invention.

Also included in the present invention are methods of co-administering the presently invented hYAK3 inhibiting compounds with further active ingredients.

### DETAILED DESCRIPTION

This invention relates to compounds of Formula (I) as described above.

The presently invented compounds of Formula (I) inhibit hYAK3 activity.

Included among the presently invented compounds of Formula I are those in which A is nitrogen.

Included among the presently invented compounds of Formula (I) are those having Formula (II):

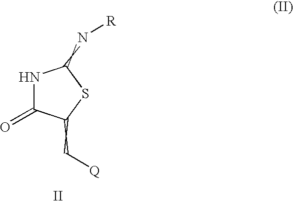

in which

R is selected form: C₁-C₁₂aryl and substituted C₁-C₁₂aryl; and

### Q is

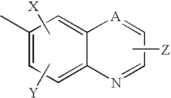

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
   where R¹⁰ is selected form hydrogen, C₁-C₄alkyl, aryl and
   trifluoromethyl, and R¹¹ and R¹² are independently selected form hydrogen, C₁-C₄alkyl, aryl and trifluoromethyl,
   provided that at least one of X, Y, Z and R⁵¹, when R⁵¹ is present, is not hydrogen;
      and/or pharmaceutically acceptable salts, hydrates, solvates and pro-drugs thereof.

Included among the presently invented compounds of Formula II are those in which A is nitrogen.

Included among the presently invented compounds of Formulas (I) and (II) are those in which:

### R is

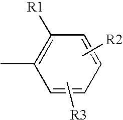 [0034] in which R¹ is hydrogen, halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —OCF₃, or —CO₂C₁₋₆alkyl; and
R² and R³ are independently hydrogen, halogen, —C₁₋₆ alkyl, substituted —C₁₋₆alkyl, C₁-C₁₂aryl, cycloalkyl, cycloalkyl containing from 1 to 4 heteroatoms, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —CO₂C₁₋₆alkyl, —NH₂, alkylamino, dialkylamino, —OCH₂(C═O)OH, —OCH₂CH₂OCH₃, —SO₂NH₂,
—S(O)₂NR⁴⁰R³⁰, where R³⁰ is selected from alkyl, cycloalkyl, substituted
cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹ C(O)R³¹, where R³¹ is selected from aryl, —Oalkyl, —Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl,
substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl, —NH(C═NH)CH₃; and

### Q is

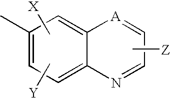

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
   where R¹⁰ is selected form hydrogen, C₁-C₄alkyl, aryl and
   trifluoromethyl, and R¹¹ and R¹² are independently selected form hydrogen, C₁-C₄alkyl, aryl and trifluoromethyl,
   provided that at least one of X, Y, Z and R⁵¹, when R⁵¹ is present, is not hydrogen;
      and/or pharmaceutically acceptable salts, hydrates, solvates and pro-drugs thereof.

Included among the presently invented compounds of Formula I and II, described immediately above, are those in which A is nitrogen.

Included among the presently invented compounds of Formulas (I) and (II) are those in which:

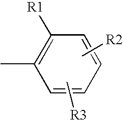

in which R1 is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl; and R2 and R3 are independently hydrogen, halogen, —C₁₋₆ alkyl, substituted —C₁₋₆ alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹ C(O)R³¹, where R³¹ is selected from aryl, —Oalkyl, —Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl,
substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl, —CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl, —CO₂C₁₋₆alkyl, —NH₂, alkylamino, dialkylamino or —NH(C═NH)CH₃;
and

### Q is

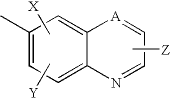

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
   where R¹⁰ is selected form hydrogen, C₁-C₄alkyl, aryl and
   trifluoromethyl, and R¹¹ and R¹² are independently selected form hydrogen, C₁-C₄alkyl, aryl and trifluoromethyl,
   provided that at least one of X, Y, Z and R⁵¹, when R⁵¹ is present, is not hydrogen;
      and/or pharmaceutically acceptable salts, hydrates, solvates and pro-drugs thereof.

Included among the presently invented compounds of Formula I and II, described immediately above, are those in which A is nitrogen.

Included among the presently invented compounds of Formulas (I) and (II) are those in which:

### R is

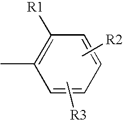 [0064] in which R1 is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl; and R2 and R3 are independently hydrogen, halogen, —C₁₋₆ alkyl,
substituted —C₁₋₆ alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H,
—S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, —Oalkyl, —Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—CO₂C₁₋₆alkyl, —NH₂, alkylamino or —NH(C═NH)CH₃;
and

### Q is

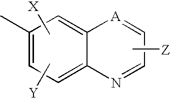

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
   where R¹⁰ is selected form hydrogen, C₁-C₄alkyl, aryl and
   trifluoromethyl, and R¹¹ and R¹² are independently selected form hydrogen, C₁-C₄alkyl, aryl and trifluoromethyl,
   provided that at least one of X, Y, Z and R⁵¹, when R⁵¹ is present, is not hydrogen;
      and/or pharmaceutically acceptable salts, hydrates, solvates and pro-drugs thereof.

Included among the presently invented compounds of Formula I and II, described immediately above, are those in which A is nitrogen.

Included among the presently invented compounds of Formulas (I) and (II) are those in which:
R is

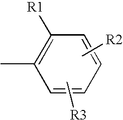

in which R1 is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl; and R2 and R3 are independently hydrogen, halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —OH, —CF₃, —CN, —CO₂H,
—S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—N R⁴¹C(O)R³¹, where R³¹ is selected from aryl, —Oalkyl, —Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR³⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—CO₂C₁₋₆alkyl, —NH₂, alkylamino, or —NH(C═NH)CH₃;
and
Q is

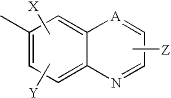

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
   where R¹⁰ is selected form hydrogen, C₁-C₄alkyl, aryl and
   trifluoromethyl, and R¹¹ and R¹² are independently selected form hydrogen, C₁-C₄alkyl, aryl and trifluoromethyl,
   provided that at least one of X, Y, Z and R⁵¹, when R⁵¹ is present, is not hydrogen;
      and/or pharmaceutically acceptable salts, hydrates, solvates and pro-drugs thereof.

Included among the presently invented compounds of Formula I and II, described immediately above, are those in which A is nitrogen.

Included among the novel compounds useful in the present invention are:

(5Z)-2-[(2,6-Dichlorophenyl)amino]-5-{[3-(4-morpholinyl)-6-quinoxalinyl]methylidene}-1,3-thiazol-4(5H)-one;

7-{(Z)-[2-[(2,6-Dichlorophenyl)amino]-4-oxo-1,3-thiazol-5(4H)-ylidene]methyl}-2(1H)-quinoxalinone;

(5Z)-2-[(2,6-Dichlorophenyl)amino]-5-{[2-(methylamino)-6-quinolinyl]methylidene}-1,3-thiazol-4(5H)-one;

(5Z)-2-[(2,6-Dichlorophenyl)amino]-5-{[4-(dimethylamino)-6-quinolinyl]methylidene}-1,3-thiazol-4(5H)-one;

(5Z)-2-[(2,6-Dichlorophenyl)amino]-5-{[4-(4-methyl-1-piperazinyl)-6-quinolinyl]methylidene}-1,3-thiazol-4(5H)-one;

(5Z)-5-[(2-Chloro-6-quinolinyl)methylidene]-2-[(2,6-dichlorophenyl)amino]-1,3-thiazol-4(5H)-one;

(5Z)-2-[(2,6-dichlorophenyl)amino]-5-[(2-methyl-6-quinolinyl)methylidene]-1,3-thiazol-4(5H)-one;

(5Z)-5-[(7-Chloro-6-quinolinyl)methylidene]-2-[(2,6-dichlorophenyl)amino]-1,3-thiazol-4(5H)-one trifluoroacetate;

(5Z)-5-[(8-Chloro-6-quinolinyl)methylidene]-2-[(2,6-dichlorophenyl)amino]-1,3-thiazol-4(5H)-one, trifluoroacetate salt;

(5Z)-2-[(2-Chlorophenyl)amino]-5-[(8-methyl-6-quinolinyl)methylidene]-1,3-thiazol-4(5H)-one;

(5Z)-5-[(5-Chloro-6-quinolinyl)methylidene]-2-[(2,6-dichlorophenyl)amino]-1,3-thiazol-4(5H)-one;

(5Z)-2-[(2,6-Dichlorophenyl)amino]-5-[(2-pentyl-6-quinolinyl)methylidene]-1,3-thiazol-4(5H)-one;

6-{(Z)-[2-[(2,6-Dichlorophenyl)amino]-4-oxo-1,3-thiazol-5(4H)-ylidene]methyl}-2(1H)-quinolinone;

(5Z)-2-[(2,6-Dichlorophenyl)amino]-5-[(2-ethyl-6-quinolinyl)methylidene]-1,3-thiazol-4(5H)-one;

(5Z)-2-[(2,6-Dichlorophenyl)amino]-5-{[2-(methyloxy)-6-quinolinyl]methylidene}-1,3-thiazol-4(5H)-one;

(5Z)-2-[(2,6-Dichlorophenyl)amino]-5-{[2-(dimethylamino)-6-quinolinyl]methylidene}-1,3-thiazol-4(5H)-one;

(5Z)-2-[(2,6-Dichlorophenyl)amino]-5-[(4-hydroxy-6-quinolinyl)methylidene]-1,3-thiazol-4(5H)-one;

(5Z)-2-[(2,6-Dichlorophenyl)amino]-5-[(3-methyl-6-quinolinyl)methylidene]-1,3-thiazol-4(5H)-one;

(5Z)-2-[(2,6-Dichlorophenyl)amino]-5-{[3-(1-methylethyl)-6-quinolinyl]methylidene}-1,3-thiazol-4(5H)-one;

(5Z)-2-[(2,6-Dichlorophenyl)amino]-5-{[4-(methylamino)-6-quinolinyl]methylidene}-1,3-thiazol-4(5H)-one;

Ethyl-4-chloro-6-{(Z)-[2-[(2,6-dichlorophenyl)amino]-4-oxo-1,3-thiazol-5(4H)-ylidene]methyl}-3-quinolinecarboxylate;

(5Z)-5-[(4-Chloro-6-quinolinyl)methylidene]-2-[(2,6-dichlorophenyl)amino]-1,3-thiazol-4(5H)-one;

(5Z)-2-[(2,6-Dichlorophenyl)amino]-5-{[4-(methyloxy)-6-quinolinyl]methylidene}-1,3-thiazol-4(5H)-one;

(5Z)-2-[(2,6-Dichlorophenyl)amino]-5-{[4-(1-piperidinyl)-6-quinolinyl]methylidene}-1,3-thiazol-4(5H)-one trifluoroacetate;

(5Z)-2-[(2,6-Dichlorophenyl)amino]-5-{[4-(4-morpholinyl)-6-quinolinyl]methylidene}-1,3-thiazol-4(5H)-one;

(5Z)-2-[(2,6-Dichlorophenyl)amino]-5-[(8-fluoro-6-quinolinyl)methylidene]-1,3-thiazol-4(5H)-one;

(5Z)-2-[(2,6-Dichlorophenyl)amino]-5-({4-[(3,3-dimethylbutyl)amino]-6-quinolinyl}methylidene)-1,3-thiazol-4(5H)-one trifluoroacetate;

Ethyl-6-{(Z)-[2-[(2,6-dichlorophenyl)amino]-4-oxo-1,3-thiazol-5(4H)-ylidene]methyl}-3-quinolinecarboxylate;

6-{(Z)-[2-[(2,6-Dichlorophenyl)amino]-4-oxo-1,3-thiazol-5(4H)-ylidene]methyl}-3-quinolinecarboxylic acid;

6-{(Z)-[2-[(2,6-Dichlorophenyl)amino]-4-oxo-1,3-thiazol-5(4H)-ylidene]methyl}-3-quinolinecarboxamide;

(5Z)-5-({4-[(2-Cyclopropylethyl)amino]-6-quinolinyl}methylidene)-2-[(2,6-dichlorophenyl)amino]-1,3-thiazol-4(5H)-one trifluoroacetate;

(5Z)-2-[(2,6-Dichlorophenyl)amino]-5-{[4-(1-pyrrolidinyl)-6-quinolinyl]methylidene}-1,3-thiazol-4(5H)-one;

(5Z)-2-[(2,6-Dichlorophenyl)amino]-5-{[3-(hydroxymethyl)-6-quinolinyl]methylidene}-1,3-thiazol-4(5H)-one;

6-{(Z)-[2-[(2,6-Dichlorophenyl)amino]-4-oxo-1,3-thiazol-5(4H)-ylidene]methyl}-N-methyl-3-quinolinecarboxamide hydrochloride;

6-{(Z)-[2-[(2,6-Dichlorophenyl)amino]-4-oxo-1,3-thiazol-5(4H)-ylidene]methyl}-N,N-dimethyl-3-quinolinecarboxamide;

(5Z)-2-[(2,6-Dichlorophenyl)amino]-5-[(4-phenyl-6-quinolinyl)methylidene]-1,3-thiazol-4(5H)-one trifluoroacetate;

(5Z)-2-[(3-Chloro-2-biphenylyl)amino]-5-[(4-phenyl-6-quinolinyl)methylidene]-1,3-thiazol-4(5H)-one trifluoroacetate;

(5Z)-2-[(2,6-Dichlorophenyl)amino]-5-[(4-methyl-6-quinolinyl)methylidene]-1,3-thiazol-4(5H)-one;

(5Z)-2-[(2,6-Dichlorophenyl)amino]-5-{[4-(1-methylethyl)-6-quinolinyl]methylidene}-1,3-thiazol-4(5H)-one;

(5Z)-2-[(2,6-Dichlorophenyl)amino]-5-[(4-ethyl-6-quinolinyl)methylidene]-1,3-thiazol-4(5H)-one;

6-{(Z)-[2-[(2,6-Dichlorophenyl)amino]-4-oxo-1,3-thiazol-5(4H)-ylidene]methyl}-4-quinolinecarbonitrile;

(5Z)-2-[(2,6-Dichlorophenyl)amino]-5-{[4-(4-pyridinyl)-6-quinolinyl]methylidene}-1,3-thiazol-4(5H)-one;

(5Z)-2-[(2,6-Dichlorophenyl)amino]-5-{[4-(3-pyridinyl)-6-quinolinyl]methylidene}-1,3-thiazol-4(5H)-one;

Ethyl-6-{(Z)-[2-[(2,6-dichlorophenyl)amino]-4-oxo-1,3-thiazol-5(4H)-ylidene]methyl}-4-(4-morpholinyl)-3-quinolinecarboxylate;

Ethyl-6-{(Z)-[2-[(2,6-dichlorophenyl)amino]-4-oxo-1,3-thiazol-5(4H)-ylidene]methyl}-4-(4-methyl-1-piperazinyl)-3-quinolinecarboxylate;

6-{(Z)-[2-[(2,6-Dichlorophenyl)amino]-4-oxo-1,3-thiazol-5(4H)-ylidene]methyl}-4-(4-morpholinyl)-3-quinolinecarboxamide trifluoroacetate;

N-{4-Chloro-3-[((5Z)-5-{[4-(4-morpholinyl)-6-quinolinyl]methylidene}-4-oxo-4,5-dihydro-1,3-thiazol-2-yl)amino]phenyl}cyclobutanecarboxamide;

N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}cyclobutanecarboxamide;

6-{(Z)-[2-[(2,6-Dichlorophenyl)amino]-4-oxo-1,3-thiazol-5(4H)-ylidene]methyl}-4-(4-methyl-1-piperazinyl)-3-quinolinecarboxamide trifluoroacetate;

(5Z)-2-[(2,6-Dichlorophenyl)amino]-5-{[4-(1-piperazinyl)-6-quinolinyl]methylidene}-1,3-thiazol-4(5H)-one;

N-{4-Chloro-3-[((5Z)-4-oxo-5-{[4-(1-piperazinyl)-6-quinolinyl]methylidene}-4,5-dihydro-1,3-thiazol-2-yl)amino]phenyl}cyclobutanecarboxamide;

Ethyl-6-{(Z)-[2-[(2,6-dichlorophenyl)amino]-4-oxo-1,3-thiazol-5(4H)-ylidene]methyl}-4-(dimethylamino)-3-quinolinecarboxylate;

Ethyl-6-{(Z)-[2-[(2,6-dichlorophenyl)amino]-4-oxo-1,3-thiazol-5(4H)-ylidene]methyl}-4-(methylamino)-3-quinolinecarboxylate;

Ethyl-6-{(Z)-[2-({2-chloro-5-[(cyclobutylcarbonyl)amino]phenyl}amino)-4-oxo-1,3-thiazol-5(4H)-ylidene]methyl}-4-(dimethylamino)-3-quinolinecarboxylate trifluoroacetate;

Ethyl-6-{(Z)-[2-({2-chloro-5-[(cyclobutylcarbonyl)amino]phenyl}amino)-4-oxo-1,3-thiazol-5(4H)-ylidene]methyl}-4-(methylamino)-3-quinolinecarboxylate;

Ethyl-6-{(Z)-[2-({2-chloro-5-[(cyclobutylcarbonyl)amino]phenyl}amino)-4-oxo-1,3-thiazol-5(4H)-ylidene]methyl}-4-(4-morpholinyl)-3-quinolinecarboxylate trifluoroacetate;

Ethyl-6-{(Z)-[2-[(2,6-dichlorophenyl)amino]-4-oxo-1,3-thiazol-5(4H)-ylidene]methyl}-4-(4-pyridinyl)-3-quinolinecarboxylate trifluoroacetate;

Ethyl-6-{(Z)-[2-({2-chloro-5-[(cyclobutylcarbonyl)amino]phenyl}amino)-4-oxo-1,3-thiazol-5(4H)-ylidene]methyl}-4-(4-pyridinyl)-3-quinolinecarboxylate trifluoroacetate;

6-{(Z)-[2-[(2,6-Dichlorophenyl)amino]-4-oxo-1,3-thiazol-5(4H)-ylidene]methyl}-4-(4-pyridinyl)-3-quinolinecarboxamide trifluoroacetate;

6-{(Z)-[2-[(2,6-Dichlorophenyl)amino]-4-oxo-1,3-thiazol-5(4H)-ylidene]methyl}-4-quinolinecarboxylic acid trifluoroacetate;

6-{(Z)-[2-[(2,6-Dichlorophenyl)amino]-4-oxo-1,3-thiazol-5(4H)-ylidene]methyl}-4-quinolinecarboxamide trifluoroacetate;

Methyl-6-{(Z)-[2-[(2,6-dichlorophenyl)amino]-4-oxo-1,3-thiazol-5(4H)-ylidene]methyl}-4-quinolinecarboxylate trifluoroacetate;

6-{(Z)-[2-[(2,6-Dichlorophenyl)amino]-4-oxo-1,3-thiazol-5(4H)-ylidene]methyl}-N-methyl-4-quinolinecarboxamide trifluoroacetate;

6-{(Z)-[2-[(2,6-Dichlorophenyl)amino]-4-oxo-1,3-thiazol-5(4H)-ylidene]methyl}-N,N-dimethyl-4-quinolinecarboxamide trifluoroacetate;

(5Z)-2-[(2,6-Dichlorophenyl)amino]-5-{[4-(4-morpholinylcarbonyl)-6-quinolinyl]methylidene}-1,3-thiazol-4(5H)-one trifluoroacetate;

(5Z)-2-[(2,6-Dichlorophenyl)amino]-5-({4-[(4-methyl-1-piperazinyl)carbonyl]-6-quinolinyl}methylidene)-1,3-thiazol-4(5H)-one trifluoroacetate;

(5Z)-2-[(2,6-Dichlorophenyl)amino]-5-{[3-(dimethylamino)-6-quinoxalinyl]methylidene}-1,3-thiazol-4(5H)-one;

(5Z)-2-[(2,6-Dichlorophenyl)amino]-5-({3-[2-(dimethylamino)ethyl]-6-quinoxalinyl}methylidene)-1,3-thiazol-4(5H)-one;

Ethyl-6-{(Z)-[2-({2-chloro-5-[(2-methylpropanoyl)amino]phenyl}amino)-4-oxo-1,3-thiazol-5(4H)-ylidene]methyl}-4-(4-pyridinyl)-3-quinolinecarboxylate;

(5Z)-2-[(2,6-Dichlorophenyl)amino]-5-{[3-(methyloxy)-6-quinoxalinyl]methylidene}-1,3-thiazol-4(5H)-one;

(5Z)-2-[(2,6-Dichlorophenyl)amino]-5-{[3-(4-methyl-1-piperazinyl)-6-quinoxalinyl]methylidene}-1,3-thiazol-4(5H)-one;

(5Z)-2-[(2,6-Dichlorophenyl)amino]-5-[(3-methyl-6-quinoxalinyl)methylidene]-1,3-thiazol-4(5H)-one;

(5Z)-2-[(2,6-Dichlorophenyl)amino]-5-[(3-phenyl-6-quinoxalinyl)methylidene]-1,3-thiazol-4(5H)-one;

(5Z)-2-[(2,6-Dichlorophenyl)amino]-5-({3-[(2-hydroxyethyl)(methyl)amino]-6-quinoxalinyl}methylidene)-1,3-thiazol-4(5H)-one;

(5Z)-2-[(2,6-Dichlorophenyl)amino]-5-({3-[[2-(dimethylamino)ethyl](methyl)amino]-6-quinoxalinyl}methylidene)-1,3-thiazol-4(5H)-one;

(5Z)-2-[(2,6-Dichlorophenyl)amino]-5-{[3-(phenylamino)-6-quinoxalinyl]methylidene}-1,3-thiazol-4(5H)-one;

N-{4-Chloro-3-[((5Z)-5-{[3-(4-morpholinyl)-6-quinoxalinyl]methylidene}-4-oxo-4,5-dihydro-1,3-thiazol-2-yl)amino]phenyl}cyclobutanecarboxamide;

(5Z)-5-[(3-Amino-6-quinoxalinyl)methylidene]-2-[(2,6-dichlorophenyl)amino]-1,3-thiazol-4(5H)-one;

N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}-2-methylpropanamide trifluoroacetate;

N-{4-Chloro-3-[((5Z)-5-{[4-(4-morpholinyl)-6-quinolinyl]methylidene}-4-oxo-4,5-dihydro-1,3-thiazol-2-yl)amino]phenyl}-2-methylpropanamide trifluoroacetate;

N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}ethanesulfonamide trifluoroacetate;

N-{4-Chloro-3-[((5Z)-5-{[4-(4-morpholinyl)-6-quinolinyl]methylidene}-4-oxo-4,5-dihydro-1,3-thiazol-2-yl)amino]phenyl}ethanesulfonamide trifluoroacetate;

Ethyl-6-{(Z)-[2-({2-chloro-5-[(2-methylpropanoyl)amino]phenyl}amino)-4-oxo-1,3-thiazol-5(4H)-ylidene]methyl}-4-(4-morpholinyl)-3-quinolinecarboxylate trifluoroacetate;

Ethyl-6-{(Z)-[2-({2-chloro-5-[(2-methylpropanoyl)amino]phenyl}amino)-4-oxo-1,3-thiazol-5(4H)-ylidene]methyl}-3-quinolinecarboxylate trifluoroacetate;

Ethyl-6-{(Z)-[2-({2-chloro-5-[(2-methylpropanoyl)amino]phenyl}amino)-4-oxo-1,3-thiazol-5(4H)-ylidene]methyl}-4-(4-methyl-1-piperazinyl)-3-quinolinecarboxylate trifluoroacetate; and

Ethyl-6-{(Z)-[2-({2-chloro-5-[(cyclobutylcarbonyl)amino]phenyl}amino)-4-oxo-1,3-thiazol-5(4H)-ylidene]methyl}-3-quinolinecarboxylate trifluoroacetate.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention and used in the methods of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazol, benzimidazol, benzothiohpene, tetrahydrobenzothiohpene and tetrazole.

The term “substituted” as used herein, unless otherwise defined, is meant that the subject chemical moiety has one or more substituents selected from the group consisting of: aryl, aryl substituted with one or more subsititents selected from alkyl, hydroxy, alkoxy, oxo, C₁-C₁₂aryl optionally substituted with one or more substituents selected from hydroxy, alkoxy oxo, cyano, amino, alkylamino, dialkylamino, alkyl and alkoxy, trifluoromethyl, —SO₂NR²¹R²², N-acylamino, —CO₂R²⁰, and halogen, cycloalkyl substituted with one or more subsititents selected from alkyl, hydroxy, alkoxy, trifluoromethyl, —SO₂NR²¹R²², amino, —CO₂R²⁰, N-acylamino and halogen, cycloalkyl containing from 1 to 4 heteroatoms substituted with one or more subsititents selected from alkyl, hydroxy, alkoxy, —SO₂NR²¹R²², amino, —CO₂R²⁰, trifluoromethyl, N-acylamino and halogen, alkoxy substituted with one or more substituents selected form alkyl, —CO₂H, hydroxyl, C₁-C₁₂aryl, alkoxy, amino and halogen, cycloalkyl, cycloalkyl containing from 1 to 4 heteroatoms, C₁-C₄alkylcycloalkyl containing from 1 to 3 heteroatomsC₁-C₄alkyl, —C(O)NHS(O)₂R²⁰, —(CH₂)_{g}NR²³S(O)₂R²⁰, hydroxyalkyl, alkoxy, —(CH₂)_{g}NR²¹R²²,—C(O)NR²¹R²², —S(O)₂NR²¹R²², —(CH₂)_{g}N(R²⁰)C(O)ₙR²⁰, —(CH₂)_{g}N═C(H)R²⁰, —C(O)R²⁰, acyloxy, —SC₁-C₆alkyl, alkyl, —OCF₃, amino, hydroxy, alkylamino, acetamide, aminoalkyl, aminoalkoxy, alkylaminoalkoxy, dialkylaminoalkoxy, alkoxyalkylamide, alkoxyC₁-C₁₂aryl, C₁-C₁₂aryl, C₁-C₁₂arylalkyl, dialkylamino, N-acylamino, aminoalkylN-acylamino, —(CH₂)_{g}C(O)OR²⁰, —(CH₂)_{g}S(O)ₙR²³, nitro, cyano, oxo, halogen, trifluoromethyloxy and trifluoromethyl;

where g is 0 to 6, n is 0 to 2, R²³ is hydrogen or alkyl, each R²⁰ is independently selected form hydrogen, alkyl, C₁-C₆alkyloxyC₁-C₆alkyl, C₁-C₄alkylC(O)OC₁-C₄alkyl, amino, alkylamino, dialkylamino, aminoC₁-C₆alkyl, alkylaminoC₁-C₆alkyl, dialkylaminoC₁-C₆alkyl, —C(O)OH, alkoxy, aryloxy, arylamino, diarylamino, arylalkylamino, aryl, aryl substituted with one or more substituents selected from oxo, hydroxyl and alkyl, arylC₁-C₄alkyl optionally substituted with one or more substituents selected from oxo, hydroxy, halogen, alkoxy and alkyl, —CH₂C(O)cycloalkyl containing from 1 to 4 heteroatoms, cycloalkylC₁-C₄alkyl, C₁-C₄alkyl substituted with cycloalkyl containing from 1 to 4 heteroatoms, cycloalkyl, cycloalkyl substituted with one or more substituents selected from oxo, hydroxyl and alkyl, cycloalkyl containing from 1 to 4 heteroatoms, cycloalkyl containing from 1 to 4 heteroatoms substituted with one or more substituents selected from oxo, hydroxyl and alkyl, and trifluoromethyl, and R²¹ and R²² are independently selected form hydrogen, alkyl, C₁-C₆alkyl substituted with one of more substituents selected from hydroxy, amino, ═NH, and ≡N, —S(O)₂aryl, —S(O)₂alkyl, C₁-C₁₂aryl, cycloalkyl containing from 1 to 4 heteroatoms, cycloalkyl containing from 1 to 4 heteroatoms substituted with one or more substituents selected from oxo, hydroxy, and alkyl, cycloalkyl, cycloalkyl substituted with one or more substituents selected from oxo, hydroxy, and alkyl, arylC₁-C₆alkyl optionally substituted with one or more substituents selected from oxo, hydroxy, and alkyl, cycloalkyl containing from 1 to 4 heteroatoms optionally substituted with one or more substituents selected from oxo, hydroxyl and alkyl, C₁-C₆alkoxy, C₁-C₄alkyloxyC₁-C₄alkyl, aryl and trifluoromethyl.

Suitably, the term “substituted” whenever used herein means that the subject chemical moiety has from one to five of the indicated substituents. Suitably, the term “substituted” whenever used herein means that the subject chemical moiety has from one to three of the indicated substituents. Suitably, the term “substituted” whenever used herein means that the subject chemical moiety has one or two of the indicated substituents.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, aminocyclohexyl, cyclobutyl, aminocyclobutyl, 4-hydroxy-cyclohexyl, 2-ethylcyclohexyl, propyl4-methoxycyclohexyl, 4-methoxycyclohexyl, 4-carboxycyclohexyl, cyclopropyl, aminocyclopentyl, and cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidine, piperazine, pyrrolidine, 3-methylaminopyrrolidine, piperazinly, tetrazole, hexahydrodiazepine and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifuloromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms.

Examples of alkyl and substituted alkyl substituents as used herein include: —CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and—C≡C—CH₃.

By the term “treating” and derivatives thereof as used herein, is meant prophylatic and therapeutic therapy.

As used herein, the crisscrossed double bond indicated by the symbol

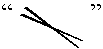

denotes Z and/or E stereochemistry around the double bond. In other words a compound of formula I or II can be either in the Z or E stereochemistry around this double bond, or a compound of formula I or II can also be in a mixture of Z and E stereochemistry around the double bond. However, in formulas I and II, the preferred compounds have Z stereochemistry around the double bond to which radical Q is attached.

The compounds of Formulas I and II naturally may exist in one tautomeric form or in a mixture of tautomeric forms. For example, for sake simplicity, compounds of formula I and II are expressed in one tautomeric form, usually as an exo form, i.e.

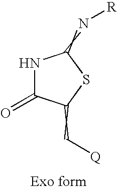

However, a person of ordinary skill can readily appreciate, the compounds of formulas I and II can also exist in endo forms.

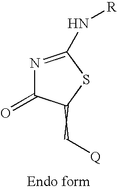

The present invention contemplates all possible tautomeric forms.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by formula I or II above as well as any wholly or partially equilibrated mixtures thereof. The present invention also covers the individual isomers of the compounds represented by the formulas above as mixtures with isomers thereof in which one or more chiral centers are inverted. Further, an example of a possible tautomer is an oxo substituent in place of a hydroxy substituent. Also, as stated above, it is understood that all tautomers and mixtures of tautomers are included within the scope of the compounds of Formula I or II.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The novel compounds of Formulas I and II are prepared as shown in Schemes I to V below, or by analogous methods, wherein the ‘Q’ and ‘R’ substituents are as defined in Formulas I and II respectively and provided that the ‘Q’ and ‘R’ substituents do not include any such substituents that render inoperative the processes of Schemes I to V. All of the starting materials are commercially available or are readily made from commercially available starting materials by those of skill in the art.

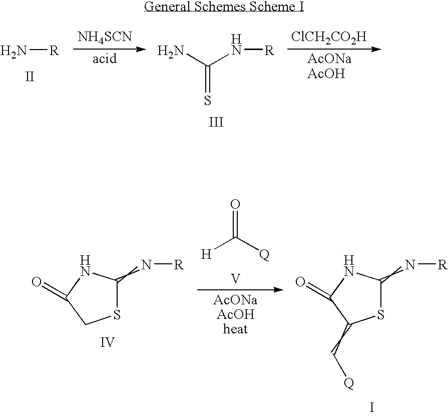

Briefly in Scheme 1, a mixture of aniline derivative of formula II (1 equivalent) and NH4SCN (about 1.3 equivalent) in an acid (typically 4N-HCl) is heated to reflux at about 110° C. for 6 hours. After cooling, the mixture is treated with H₂O, which process usually forms a solid, followed by desiccation in vacuo to give a compound of formula III.

A mixture of formula III compound, ClCH₂CO₂H (1 equivalent), and AcONa (1 equivalent) in AcOH is heated to reflux at around 110° C. for about 4 h. The mixture is poured onto water thereby a solid is typically formed, which is isolated by filtration. The solid is washed with a solvent such as MeOH to afford a compound of formula IV.

A mixture of formula IV compound, an aldehyde of formula V (1 equivalent), AcONa (3 equivalent) in AcOH is heated to reflux at about 110° C. for about 10 to 48 hours. After cooling, a small portion of water is added until the solid forms. The solid is filtered and washed with a solvent such as MeOH, followed by desiccation in vacuo to afford a target product of Formula I.

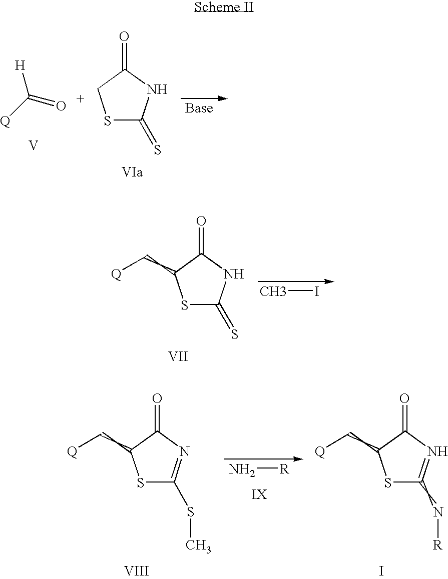

Briefly in Scheme 2, a mixture of an aldehyde of formula V (1 equivalent), Rhodanine (1 equivalent), sodium acetate (about 3 equivalents), and acetic acid is heated at around 110° C. for about 48 h. The reaction mixture is cooled to room temperature to afford a product of formula VII.

Then, to a room temperature suspension of VII (1 equivalent) in a suitable solvent such as ethanol is added Hunig's base (about 2 equivalents) followed by iodomethane (about 5 equivalents). The resultant suspension is stirred at room temperature for 3.5 h to yield a compound of VIII.

To a mixture of VIII (1 equivalent) and MS4A powder is added an amine of formula IX (1˜2 equivalent) and ethanol (dehydrated). The mixture is heated by microwave (SmithSynthesizer-Personal Chemistry) at about 110° C. for about 1200 seconds. The desired product of Formula I is obtained in about 20˜90% yield after purification.

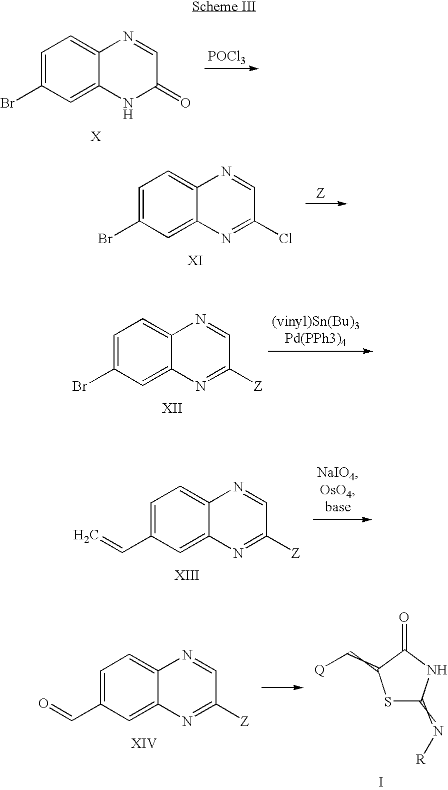

Briefly in Scheme III, compound X can be treated with a chlorinating agent such as phosphorous oxychloride or thionyl chloride to afford chloro compound XI which can be treated with a nucleophile such as an amine, alcohol or thiol to afford compound XII. Coupling of compound XII with a vinyl stannane such as tributlyvinyltin in the presence of a palladium catalyst such as tetrakistriphenylphosphinopalladium gives compound XIII which can be treated with a dihydroxylating reagent such as sodium periodate and an oxidative cleavage reagent such as osmium tetraoxide in the presence of a base such as 2,6-lutidine to afford aldehyde XIV. Condensation of compound XIV with an appropriately substutiuted thiazolidin-4-one in the presence of a base such as piperidine, sodium acetate or morpholine in a solvent such as ethanol or acetic acid with heating at a temperature such as 110° C. or 150° C. in a microwave reactor or in a sealed vessel affords compounds of Example I.

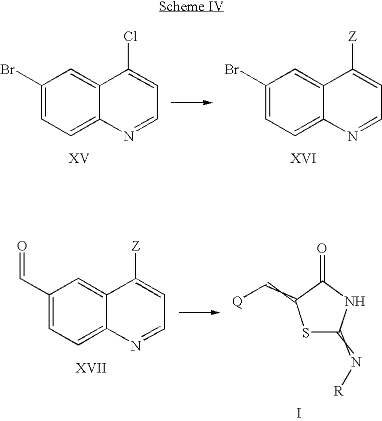

Briefly in Scheme IV, compound XV can be treated with a nucleophile such as an amine, alcohol or thiol to afford compound XVI. Treatment of compound XVI with an organolithium reagent such as butyl lithium followed by addition of a tertiary formamide such as N,N-dimethylformamide affords compound XVII. Condensation of compound XVII with an appropriately substutiuted thiazolidin-4-one in the presence of a base such as piperidine, sodium acetate or morpholine in a solvent such as ethanol or acetic acid with heating at a temperature such as 110° C. or 150° C. in a microwave reactor or in a sealed vessel affords compounds of Example I.

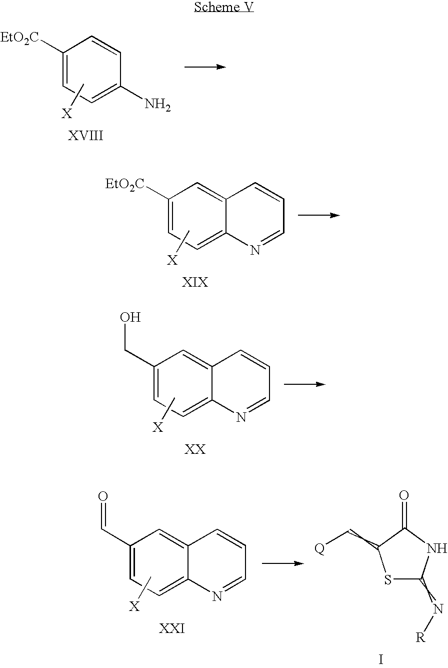

Briefly in Scheme V, compound XVIII is heated with a mixture of glycerol, sulphuric acid and sodium 3-nitrobenzenesulphonate to afford compound XIX which is reduced with a hydride source such as lithium aluminum hydride in a solvent such as THF or diethyl ether to afford compound XX. Compound XX is then oxidized using an oxidizing agent such as manganese dioxide or pyridinium dichromate in a asuitable solvent such as acetonitrile or dichloromethane to afford compound XXI. Condensation of compound XXI with an appropriately substutiuted thiazolidin-4-one in the presence of a base such as piperidine, sodium acetate or morpholine in a solvent such as ethanol or acetic acid with heating at a temperature such as 110° C. or 150° C. in a microwave reactor or in a sealed vessel affords compounds of Example I.

In Schemes I to V, the meaning of R, X, Z and Q are as defined in Formula I.

In other embodiments, additional compounds of the invention can also be synthesized whereby a compound of Formula I is first made by a process of Scheme 1 or 2 (or a variant thereof), and Q and R radicals in compounds of Formula I thus made are further converted by routine organic reaction techniques into different Q and R groups.

By the term “co-administering” and derivatives thereof as used herein is meant either simultaneous administration or any manner of separate sequential administration of a hYAK3 inhibiting compound, as described herein, and a further active ingredient or ingredients, known to be useful in treating diseases of the hematopoietic system, particularly anemias, including EPO or a derivative thereof. The term further active ingredient or ingredients, as used herein, includes any compound or therapeutic agent known to or that demonstrates advantageous properties when administered to a patient in need of treatment for diseases of the hematopoietic system, particularly anemias, and any compound or therapeutic agent known to or that demonstrates advantageous properties when administered in combination with a hYAK3 inhibiting compound. Preferably, if the administration is not simultaneous, the compounds are administered in a close time proximity to each other. Furthermore, it does not matter if the compounds are administered in the same dosage form, e.g. one compound may be administered topically and another compound may be administered orally.

Because the pharmaceutically active compounds of the present invention are active as hYAK3 inhibitors they exhibit therapeutic utility in treating diseases of the hematopoietic system, particularly anemias.

The pharmaceutically active compounds within the scope of this invention are useful as hYAK inhibitors in mammals, particularly humans, in need thereof.

The present invention therefore provides a method of treating diseases of the hematopoietic system, particularly anemias and other conditions requiring hYAK inhibition, which comprises administering an effective compound of Formula (I) or a pharmaceutically acceptable salt, hydrate, solvate or pro-drug thereof. The compounds of Formula (I) also provide for a method of treating the above indicated disease states because of their ability to act as hYAK inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a hYAK inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular hYAK inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing hYAK inhibitory activity in mammals, including humans, comprises administering to a subject in need of such activity an effective hYAK inhibiting amount of a pharmaceutically active compound of the present invention.

The invention also provides for the use of a compound of Formula (I) in the manufacture of a medicament for use as a hYAK inhibitor.

The invention also provides for the use of a compound of Formula (I) in the manufacture of a medicament for use in therapy.

The invention also provides for the use of a compound of Formula (I) in the manufacture of a medicament for use in treating diseases of the hematopoietic system, particularly anemias.

The invention also provides for a pharmaceutical composition for use as a hYAK inhibitor which comprises a compound of Formula (I) and a pharmaceutically acceptable carrier.

The invention also provides for a pharmaceutical composition for use in the treatment of diseases of the hematopoietic system, particularly anemias which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat diseases of the hematopoietic system, particularly anemias, or compounds known to have utility when used in combination with a hYAK inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

For ease of illustration, the regiochemistry around the double bonds in the chemical formulas in the Examples are drawn as fixed for ease of representation; however, a skilled in the art will readily appreciate that the compounds will naturally assume more thermodynamically stable structure around the C═N (the imine) double bond if it exits as exo form. Further compounds can also exit in endo form. As stated before, the invention contemplates both endo and exo forms as well as both regioisomers around the exo imine bond. Further it is intended that both E and Z isomers are encompassed around the C═C double bond.

### Experimental Details

The compounds of Examples 1 to 86 are readily made according to Schemes I to V or by analogous methods.

### EXAMPLE 1

### (5Z)-2-[(2,6-Dichlorophenyl)amino]-5-{[3-(4-morpholinyl)-6-quinoxalinyl]methylidene}-1,3-thiazol-4(5H)-one

a) 7-Bromo-2-chloroquinoxaline. A solution of 7-bromo-2(1H)-quinoxalinone (prepared by the method of Linda, P; Marino, G. Ric. Sci., Rend. Sez A. [2] 1963, 3, 225-228) (0.900 g, 4.00 mmol) was treated with phosphorus oxychloride (5.0 mL). After heating to reflux overnight, the reaction mixture was poured into ice water, extracted thrice with ethyl acetate, dried over magnesium sulfate, filtered, and concentrated in vacuo to give the title compound as an off-white solid (0.905 g, 93%). 1H NMR (400 MHz, DMSO-d₆) δ ppm 9.05 (s, 1 H) 8.34 (d, J=2.0 Hz, 1 H) 8.11 (d, J=9.0 Hz, 1 H) 8.05 (dd, J=8.9, 2.0 Hz, 1 H). MS(ES+) m/e 243/245 [M+H]⁺.
b) 7-Bromo-2-(4-morpholinyl)quinoxaline. A solution of the compound from Example 1a) (5.00 g; 20.5 mmol) and morpholine (1.80 mL; 20.5 mmol) in methanol (4.0 mL) was stirred and heated to 150° C. for 20 min. in a Biotage Initiator microwave synthesizer. Concentration in vacuo and recrystallization from CH₂Cl₂ provided the title compound as a light yellow crystalline solid (5.75 g, 95%). 1H NMR (400 MHz, CHLOROFORM-d) δ ppm 8.45 (s, 1 H) 7.76 (d, J=2.0 Hz, 1 H) 7.64 (d, J=8.6 Hz, 1 H) 7.38 (dd, J=8.7, 2.1 Hz, 1 H) 3.76-3.84 (m, 4 H) 3.67-3.71 (m, 4 H). MS(ES+) m/e 294/296 [M+H]⁺.
c) 7-Ethenyl-2-(4-morpholinyl)quinoxaline. A solution of the compound from Example 1b) (0.362 g; 1.23 mmol), tributyl(vinyl)tin (0.395 mL; 1.35 mmol), and tetrakis(triphenylphosphine)palladium(0) (0.014 g; 0.012 mmol) in dioxane (2.0 mL) was stirred and heated to 150° C. for 20 min. in a Biotage Initiator microwave synthesizer. Concentration in vacuo and purification via flash column chromatography (10-50% ethyl acetate in hexanes) provided the title compound as a yellow solid (0.276 g, 93%). 1H NMR (400 MHz, CHLOROFORM-d) δ ppm 8.51 (s, 1 H) 7.84 (d, J=8.6 Hz, 1 H) 7.66 (d, J=1.5 Hz, 1 H) 7.55 (dd, J=8.5, 1.9 Hz, 1 H) 6.86 (dd, J=17.4, 10.9 Hz, 1 H) 5.92 (d, J=17.7 Hz, 1 H) 5.39 (d, J=11.1 Hz, 1 H) 3.83-4.01 (m, 4 H) 3.69-3.83 (m, 4 H). MS(ES+) m/e 242 [M+H]⁺.
d) 3-(4-Morpholinyl)-6-quinoxalinecarbaldehyde. A solution of the compound from Example 1c) (0.464 g; 1.92 mmol) in dioxane (6.0 mL) and water (2.0 mL) was treated with osmium tetroxide (0.482 mL; 2.5% soln. in t-BuOH; 0.038 mmol), 2,6-lutidine (0.448 mL; 3.85 mmol), and sodium periodate (0.825 g; 3.85 mmol). After stirring 30 min. at ambient temperature, the reaction was quenched with water, extracted thrice with CH₂Cl₂, dried over magnesium sulfate, filtered, and concentrated in vacuo. Purification via flash column chromatography (10-60% ethyl acetate in hexanes) provided the title compound as a light yellow solid (0.425 g, 91%). 1H NMR (400 MHz, CHLOROFORM-d) δ ppm 10.16 (s, 1 H) 8.65 (s, 1 H) 8.16 (d, J=1.5 Hz, 1 H) 7.98 (d, J=8.4 Hz, 1 H) 7.89 (dd, J=8.4, 1.8 Hz, 1 H) 3.85-3.93 (m, 4 H) 3.78-3.85 (m, 4 H). MS(ES+) m/e 244 [M+H]⁺.
e) (5Z)-2-[(2,6-Dichlorophenyl)amino]-5-{[3-(4-morpholinyl)-6-quinoxalinyl]methylidene}-1,3-thiazol-4(5H)-one. A solution of the compound from Example 1d) (0.263 g; 1.01 mmol), 2-[(2,6-dichlorophenyl)amino]-1,3-thiazol-4(5H)-one (0.245 g; 1.01 mmol) and piperidine (0.100 mL; 1.01 mmol) in ethanol (2.0 mL) was stirred and heated at 150° C. for 30 min. in a Biotage Initiator microwave synthesizer. The reaction mixture was then cooled, poured into 1M aqueous hydrochloric acid (5.0 mL), filtered, and washed with water to give the title compound as a brown solid (0.421 g; 86%). 1H NMR (400 MHz, CHLOROFORM-d) δ ppm 8.55 (s, 1 H) 7.88 (d, J=8.6 Hz, 1 H) 7.77 (s, 1 H) 7.72 (d, J=0.8 Hz, 1 H) 7.47 (dd, J=8.6, 1.3 Hz, 1 H) 7.39 (d, J=8.1 Hz, 2 H) 7.07 (t, J=8.1 Hz, 1 H) 4.78 (s, 1 H) 3.82-3.97 (m, 4 H) 3.74-3.81 (m, 4 H). MS(ES+) m/e 486 [M+H]⁺.

### EXAMPLE 2

### 7-{(Z)-[2-[(2,6-Dichlorophenyl)amino]-4-oxo-1,3-thiazol-5(4H)-ylidene]methyl}-2(1H)-quinoxalinone

a) 7-Ethenyl-2(1H)-quinoxalinone. A solution of 7-bromo-2(1H)-quinoxalinone (prepared by the method of Linda, P; Marino, G. Ric. Sci., Rend. Sez. A. [2] 1963, 3, 225-228)(1.04 g; 6.22 mmol), tributyl(vinyl)tin (2.0 mL; 6.84 mmol), and tetrakis(triphenylphosphine)palladium(0) (0.072 g; 0.062 mmol) in dioxane (10.0 mL) was stirred and heated to 150° C. for 20 min. in a Biotage Initiator microwave synthesizer. Concentration in vacuo and purification via flash column chromatography (20-50% ethyl acetate in hexanes) provided the title compound as a yellow solid (1.05 g, 98%). 1H NMR (400 MHz, DMSO-d₆) δ ppm 12.43 (s, 1 H) 8.12 (s, 1 H) 7.74 (d, J=8.3 Hz, 1 H) 7.48 (dd, J=8.3, 1.8 Hz, 1 H) 7.28 (d, J=1.8 Hz, 1 H) 6.84 (dd, J=17.7, 10.9 Hz, 1 H) 5.94 (d, J=17.7 Hz, 1 H) 5.43 (d, J=11.4 Hz, 1 H). MS(ES+) m/e 173 [M+H]⁺.
b) 3-Oxo-3,4-dihydro-6-quinoxalinecarbaldehyde. A solution of the compound from Example 2a) (0.075 g; 0.436 mmol) in dioxane (3.0 mL) and water (1.0 mL) was treated with osmium tetroxide (0.109 mL; 2.5% soln. in t-BuOH; 0.009 mmol), 2,6-lutidine (0.101 mL; 0.871 mmol), and sodium periodate (0.186 g; 0.871 mmol). After stirring 30 min. at ambient temperature, the reaction was quenched with water, extracted thrice with CH₂Cl₂, dried over magnesium sulfate, filtered, and concentrated in vacuo to give the title compound as a light yellow solid (0.074 g, 98%). 1H NMR (400 MHz, DMSO-d₆) δ ppm 12.70 (s, 1 H) 10.09 (s, 1 H) 8.31 (d, J=2.3 Hz, 1 H) 7.98 (d, J=8.1 Hz, 1 H) 7.80 (dd, J=8.1, 1.5 Hz, 1 H) 7.79 (s, 1 H). MS(ES+) m/e 175 [M+H]⁺.
c) 7-{(Z)-[2-[(2,6-Dichlorophenyl)amino]-4-oxo-1,3-thiazol-5(4H)-ylidene]methyl}-2 (1H)-quinoxalinone. A solution of the compound from Example 2b) (0.067 g; 0.383 mmol), 2-[(2,6-dichlorophenyl)amino]-1,3-thiazol-4(5H)-one (0.100 g; 0.383 mmol) and piperidine (0.038 mL; 0.383 mmol) in ethanol (2.0 mL) was stirred and heated at 150° C. for 30 min. in a Biotage Initiator microwave synthesizer. The reaction mixture was then cooled, poured into 1M aqueous hydrochloric acid (5.0 mL), filtered, and washed with water to give the title compound as an orange solid (0.129 g; 81%). 1H NMR (400 MHz, DMSO-d₆) δ ppm 13.05 (s, 1 H) 12.45 (s, 1 H) 8.18 (d, J=2.3 Hz, 1 H) 7.83 (d, J=8.3 Hz, 1 H) 7.76 (s, 1 H) 7.58 (d, J=8.1 Hz, 2 H) 7.47 (dd, J=8.3, 1.5 Hz, 1 H) 7.37 (d, J=0.8 Hz, 1 H) 7.24 (t, J=8.1 Hz, 1 H). MS(ES+) m/e 417 [M+H]⁺.

### EXAMPLE 3

### (5Z)-2-[(2,6-Dichlorophenyl)amino]-5-{[2-(methylamino)-6-quinolinyl]methylidene}-1,3-thiazol-4(5H)-one

a) (5Z)-5-[(2-Chloro-6-quinolinyl)methylidene]-2-[(2,6-dichlorophenyl)amino]-1,3-thiazol-4(5H)-one. A solution of 2-chloro-6-quinolinecarbaldehyde (S. Inglis et. al., J. Med. Chem., 2004, 47(22), 5405; 20 mg, 0.1 mmol), 2-[(2,6-dichlorophenyl)amino]-1,3-thiazol-4(5H)-one (26 mg, 0.1 mmol) and piperazine (0.010 mL, 0.1 mmol) in ethanol (2.0 mL) was stirred and heated at 150° C. for 30 min. in a Biotage Initiator microwave synthesizer. The reaction mixture was then cooled, poured into 1M aqueous hydrochloric acid (5.0 mL), filtered, and washed with water to give the title compound as a yellow solid (18 mg, 40%). MS(ES+) m/e 433[M+H]⁺. 1H NMR (400 MHz, CHLOROFORM-d) δ ppm 8.15 (d, J=8.84 Hz, 1 H) 8.04 (d, J=8.84 Hz, 1 H) 7.89 (s, 1 H) 7.87 (s, 1 H) 7.76 (dd, J=8.84, 1.77 Hz, 1 H) 7.38-7.46 (m, 1 H) 7.12 (t, J=8.08 Hz, 1 H).
b) (5Z-2-[(2,6-Dichlorophenyl)amino]-5-{[2-(methylamino)-6-quinolinyl]methylidene}-1,3-thiazol-4(5H)-one. To a solution of compound from Example 3a) (10 mg, 0.023 mmol) in MeOH (2 mL) was added CH₃NH₂ (2.0 M in MeOH, 2 mL, 4 mmol). The mixture was refluxed for 5 days, cooled and purified by HPLC (YMC HPLC column 75×30 mml.D. 0-100% acetonitrile (0.1% TFA) in water (0.1% TFA)) to give the title compound as yellow solid (4 mg, 41%). MS(ES+) m/e 430 [M+H]⁺. 1H NMR (400 MHz, DMSO-d₆) δ ppm 13.01 (s, 1 H) 9.82 (s, 1 H) 8.29 (d, J=9.35 Hz, 1 H) 7.95-8.07 (m, 2 H) 7.83 (d, J=8.59 Hz, 1 H) 7.80 (s, 1 H) 7.57 (d, J=8.08 Hz, 2 H) 7.24 (t, J=8.21 Hz, 1 H) 7.05 (d, J=9.09 Hz, 1 H) 3.10 (d, J=4.29 Hz, 3 H).

### EXAMPLE 4

### (5Z)-2-[(2,6-Dichlorophenyl)amino]-5-{[4-(dimethylamino)-6-quinolinyl]methylidene}-1,3 -thiazol-4(5H)-one

a) 6-Bromo-N,N-dimethyl-4-quinolinamine. To a solution of 6-bromo-4-chloroquinoline (200 mg, 0.41 mmol) (prepared by the method of Ai Jeng Lin, J. Med. Chem, 1978, 21, 268) in dry MeOH (2.0 mL) was added dimethylamine hydrochloride (67 mg, 0.82 mmol) and N,N-diisopropylethylamine (0.14 mL, 0.82 mmol). The mixture was heated at 150° C. in a Biotage Initiator microwave synthesizer for 20 minutes, then cooled and quenched with H₂O. The resulting mixture was extracted by CH₂Cl₂. The extact was dried over MgSO₄, filtered, concentrated in vacuo and purified by flash chromatography (0-10% MeOH in CH₂Cl₂) to afford colorless oil (130 mg, 62%). MS(ES+) m/e 252[M+H]⁺. 1H NMR (400 MHz, CHLOROFORM-d) δ ppm 8.61 (d, J=4.29 Hz, 1 H) 8.17 (d, J=2.02 Hz, 1 H) 7.87 (d, J=8.84 Hz, 1 H) 7.66 (dd, J=8.97, 2.15 Hz, 1 H) 6.71 (d, J=5.31 Hz, 1 H) 2.93-3.02 (m, 6 H).
b) 4-(Dimethylamino)-6-quinolinecarbaldehyde. To a solution of compound from Example 4a) (130 mg, 0.52 mmol) in dry THF at −78° C. was added n-BuLi (1.6 M in hexane, 0.32 mL, 0.52 mmol), then DMF (0.080 mL, 1.04 mmol). The mixture was warmed up to the room temperature, quenched by saturated NH₄Cl and extracted by ethyl acetate. The extract was dried over MgSO₄, filtered and concentrated in vacue. The residue was purified by flash chromatography (0-100% ethyl acetate in hexanes) to afford brown solid (40 mg, 40%). MS(ES+) m/e 201 [M+H]⁺.
c) (5Z)-2-[(2,6-dichlorophenyl)amino]-5-{[4-(dimethylamino)-6-quinolinyl]methylidene}-1,3-thiazol-4(5H)-one. To a solution of compound from Example 4b) (37.2 mg, 0.2 mmol) in ethanol (2 mL) was added 2-[(2,6-dichlorophenyl)amino]-1,3-thiazol-4(5H)-one (52 mg, 0.2 mmol) and piperidine ( 0.040 mL, 0.4 mmol). The mixture was stirred and heated at 150° C. for 30 min. in a Biotage Initiator microwave synthesizer. The solvent was removed in vacue. The residue was purified by flash chromatography (0-10% MeOH in CH₂Cl₂) to afford the title compound as yellow solid (25 mg, 38%). MS(ES+) m/e 443 [M+H]⁺. 1H NMR (400 MHz, DMSO-d₆) δ ppm 8.54 (d, J=5.81 Hz, 1 H) 8.29-8.38 (m, 1 H) 7.90-8.02 (m, 2 H) 7.78 (d, J=8.59 Hz, 1 H) 7.58 (d, J=8.08 Hz, 2 H) 7.24 (t, J=8.08 Hz, 1 H) 6.90 (d, J=5.81 Hz, 1 H) 3.15 (s, 6 H).

### EXAMPLE 5

### (5Z)-2-[(2,6-Dichlorophenyl)amino]-5-{[4-(4-methyl-1-piperazinyl)-6-quinolinyl]methylidene}-1,3-thiazol-4(5H)-one

a) 4-Chloro-6-ethenylquinoline. A solution of the 6-bromo-4-chloroquinoline (4 g; 16.5 mmol), tributyl(vinyl)tin (4.8 ml, 16.5 mmol), and tetrakis(triphenylphosphine)palladium(0) (0.190g; 0.16 mmol) in dioxane (15.0 mL) was stirred and heated to 150° C. for 20 min. a Biotage Initiator microwave synthesizer. Concentration in vacuo and purification via flash column chromatography (0-100% ethyl acetate in hexanes) provided the title compound as a yellow solid (2.5 g,80%). MS(ES+) m/e 190 [M+H]⁺. 1H NMR (400 MHz, CHLOROFORM-d) δ ppm 8.72 (d, J=4.55 Hz, 1 H) 8.02-8.13 (m, 2 H) 7.91 (dd, J=8.72, 1.89 Hz, 1 H) 7.47 (d, J=4.80 Hz, 1 H) 6.93 (dd, J=17.68, 10.86 Hz, 1 H). 5.95 (d, J=17.43 Hz, 1 H) 5.45 (d, J=11.12 Hz, 1 H).
b) 4-Chloro-6-quinolinecarbaldehyde. A solution of the compound from Example 5a) (5.36 g, 28.35 mmol) in dioxane (15.0 mL) and water (5.0 mL) was treated with osmium tetroxide (7.1 mL, 2.5% soln. in t-BuOH, 0.57 mmol), 2, 6-lutidine (6.6 mL, 56.7 mmol), and sodium periodate (24.3 g, 113.4 mmol). After stirring 2 hours at ambient temperature, the reaction was quenched with water, extracted twice with CH₂Cl₂, dried over magnesium sulfate, filtered, and concentrated in vacuo. Purification via flash column chromatography (0-100% ethyl acetate in hexanes) provided the title compound as a white solid (4.5 g, 84%). MS(ES+) m/e 192 [M+H]⁺. 1H NMR (400 MHz, CHLOROFORM-D) δ ppm 10.27 (s, 1 H) 8.93 (d, J=4.55 Hz, 1 H) 8.77 (s, 1 H) 8.19-8.34 (m, 2 H) 7.62 (d, J=4.80 Hz, 1 H).
c) (5Z)-2-[(2,6-Dichlorophenyl)amino]-5-{[4-(4-methyl-1-piperazinyl)-6-quinolinyl]methylidene}-1,3-thiazol-4(5H)-one hydrochloride. A solution of the compound from Example 5b) (200 mg, 1.05 mmol), 2-[(2,6-dichlorophenyl)amino]-1,3-thiazol-4(5H)-one (272 mg, 1.05 mmol) and 1-methylpiperazine (0.23 mL, 2.1 mmol) in ethanol (2.0 mL) was stirred and heated at 150° C. for 30 min. in a Biotage Initiator microwave synthesizer. The reaction mixture was then cooled, poured into 1M aqueous hydrochloric acid (5.0 mL), filtered, and washed with water to give the title compound as a yellow solid (0.34 g; 65%). MS(ES+) m/e 498 [M+H]⁺. 1H NMR (400 MHz, DMSO-d₆) δ ppm 13.10 (s, 1 H) 12.00 (s, 1 H) 8.80 (s, 1 H) 8.31 (s,1 H) 8.22 (s, 1 H) 8.05 (s, 1 H) 7.97 (s, 1 H) 7.55 (d, J=1.52 Hz, 2 H) 7.35 (s, 1 H) 7.21 (s, 1 H) 4.24 (s, 2 H) 3.86 (s, 2 H) 3.16-3.54 (m, 4 H) 2.79 (s, 3 H).

### EXAMPLE 6

### (5Z)-5-[(2-Chloro-6-quinolinyl)methylidene]-2-[(2,6-dichlorophenyl)amino]-1,3-thiazol-4(5H)-one

a) (2E)-N-(4-Bromophenyl)-3-(ethyloxy)-2-propenamide. (2E)-3-(Ethyloxy)-2-propenoyl chloride (5.0 g, 41 mmol) was added dropwise to a solution of 4-bromoaniline (5.8 g, 34 mmol) and pyridine (11.3 g, 123 mmol) in CH₂Cl₂ (50 mL). The mixture was kept stirring at ambient temperature overnight, quenched with ice water and extracted with ethyl acetate. The extract was dried over magnesium sulfate, concentrated in vacuo, and purified via flash column chromatography (50-100% ethyl acetate in hexanes) to give the title compound as a light yellow solid (6.0 g, 65%). MS(ES+) m/e 271 [M+H]⁺.
b) 6-Bromo-2(1H-quinolinone. The solution of the compound from Example 6a) (6.0 g, 22.2 mmol) in concentrated H₂SO₄ (30 mL) was heated in 100° C. overnight. The mixture was cooled to ambient temperature and quenched with ice water. The precipitate was filtered, washed with water and dried in vacuo to afford the title compound as a white solid (4.8 g, 97%). MS(ES+) m/e 225 [M+H]⁺.
c) 6-Bromo-2-chloroquinoline. The solution of the compound from Example 6b) (3.0 g, 13 mmol) in POCl₃ (25 mL) was refluxed for an hour. The mixture was cooled to the ambient temperature and quenched with ice water. The precipitate was filtered, washed with water and dried in vacuo to afford the title compound as a yellow solid. (2.7 g, 85%). 1H NMR (400 MHz, CHLOROFORM-d) δ ppm 8.06 (d, J=8.34 Hz, 1 H) 8.02 (d, J=2.02 Hz, 1 H) 7.93 (d, J=9.09 Hz, 1 H) 7.84 (dd, J=9.09, 2.27 Hz, 1 H) 7.44 (d, J=8.59 Hz, 1 H).
d) 2-Chloro-6-quinolinecarbaldehyde. n-Butyllithium (1.6 M in hexane, 0.5 mL, 0.8 mmol) was added the solution of the compound from Example 6c) (200 mg, 0.8 mmol) in dry THF (5 mL) at −78° C. The mixture was kept stirring at −78° C. for an hour, then DMF (76 μL, 0.98 mmol) was added. The product was let to raise the ambient temperature, quenched by saturated NH₄Cl solution, extracted with ethyl acetate. The extract was dried over magnesium sulfate, concentrated in vacuo and purified via flash column chromatography (0-100% ethyl acetate in hexanes) to give the title compound as a white solid (50 mg, 31%). 1H NMR (400 MHz, CHLOROFORM-d) δ ppm 10.22 (s, 1 H) 8.38 (d, J=1.77 Hz, 1 H) 8.30 (d, J=8.59 Hz, 1 H) 8.25 (dd, J=8.59, 1.77 Hz, 1 H) 8.16 (d, J=9.35 Hz, 1 H)7.54 (d, J=8.59 Hz, 1 H).
e) 2-[(2,6-Dichlorophenyl)amino]-1,3-thiazol-4(5H)-one. A suspension of N-(2,6-dichlorophenyl)thiourea (103.7 g; 0.469 mol.) and chloroacetic acid (48.8 g; 0.516 mol.) in glacial acetic acid (600 mL) was stirred and heated under reflux for 2 h. The stirred mixture was allowed to cool to 40° C. then treated dropwise with water (1 L) during which a pale-yellow precipitate formwed. The suspension was then filtered and the precipitate washed with water (1 L) to afford the title compound (94.0 g; 79%) as a yellow solid. ¹H NMR (400 MHz, DMSO-d₆) δ ppm 4.10 (s, 2 H) 7.14 (t, J=8.08 Hz, 1 H) 7.49 (d, J=8.08 Hz, 2 H) 12.23 (s, 1 H).
f) (5Z)-5-[(2-Chloro-6-quinolinyl)methylidene]-2-[(2,6-dichlorophenyl)amino]-1,3-thiazol-4(5H)-one. The mixture of the compound from Example 6d) (20 mg, 0.1 mmol), 2-[(2,6-dichlorophenyl)amino]-1,3-thiazol-4(5H)-one (26 mg, 0.1 mmol), piperidine (10 μL, 0.1 mmol) in ethanol (2 mL) was heated to 150° C. for 20 minutes in a Biotage Initiator microwave synthesizer. The product was cooled to the ambient temperature, concentrated in vacuo and purified via flash column chromatography (0-10% methanol in methylene chloride) to give the title compound as a yellow solid (18 mg, 40%). MS(ES+) m/e 433 [M+H]⁺. 1H NMR (400 MHz, CHLOROFORM-d) δ ppm 8.15 (d, J=8.84 Hz, 1 H) 8.04 (d, J=8.84 Hz, 1 H) 7.89 (s, 1 H) 7.87 (s, 1 H) 7.76 (dd, J=8.84, 1.77 Hz, 1 H) 7.38-7.46 (m, 1 H) 7.12 (t, J=8.08 Hz, 1 H)

### EXAMPLE 7

### (5Z)-2-[(2,6-dichlorophenyl)amino]-5-[(2-methyl-6-quinolinyl)methylidene]-1,3-thiazol-4(5H)-one.

a) 6-Bromo-2-methylquinoline. A mixture of 4-bromoaniline (1 g, 5.8 mmol), (2E)-2-butenal (412 mg, 5.8 mmol), toluene (10 mL) and 6 N HCl (20 mL) was refluxed overnight. The aqueous layer was separated, neutralized to PH=7, extracted with ethyl acetate. The extract was dried over magnesium sulfate, concentrated in vacuo and purified via flash column chromatography (0-100% ethyl acetate in hexanes) to give the title compound as a yellow solid (850 mg, 66%). 1H NMR (400 MHz, CHLOROFORM-d) δ ppm 8.00 (d, J=8.59 Hz, 1 H) 7.92-7.98 (m, 2 H) 7.77 (dd, J=8.97, 2.15 Hz, 1 H) 7.34 (d, J=8.34 Hz, 1 H) 2.77 (s, 3 H).
b) 2-Methyl-6-quinolinecarbaldehyde. The title compound was made by following the method in Example 6d) as a yellow solid (20%). The compound from Example 7a) was used in place of the compound from Example 6c). 1H NMR (400 MHz, CHLOROFORM-d) δ ppm 10.20 (s, 1 H) 8.33 (s, 1 H) 8.25 (d, J=8.34 Hz, 1 H) 8.17-8.20 (m, 2 H) 7.44 (d, J=8.34 Hz, 1 H) 2.84 (s, 3 H).
c) (5Z)-2-[(2,6-Dichlorophenyl)amino]-5-[(2-methyl-6-quinolinyl)methylidene]-1,3-thiazol-4(5H)-one. The title compound was made by following the procedure in Example 6f) as a yellow solid (13%). The compound from Example 7b) was used in place of the compound from Example 6d). 1H NMR (400 MHz, DMSO-d₆) δ ppm 8.26 (s, 1 H) 7.80-8.05 (m, 3 H) 7.27-7.49 (m, 4 H) 6.99 (s, 1 H) 2.65 (s, 3 H)

### EXAMPLE 8

### (5Z)-5-[(7-Chloro-6-quinolinyl)methylidene]-2-[(2,6-dichlorophenyl)amino]-1,3-thiazol-4(5H)-one trifluoroacetate

a) Ethyl 7-chloro-6-quinolinecarboxylate and ethyl 5-chloro-6-quinolinecarboxylate. A solution of 4-amino-2-chlorobenzoic acid (17.1 g; 0.1 mol.), glycerol (41.4 g; 0.45 mol.), sodium 3-nitrobenzenesulfonate (45.0 g; 0.2 mol.) and 75% aq. sulfuric acid (250 mL) was stirred and heated at 100° C. for 3 h then at 140° C. for 1 h. The mixture was allowed to cool to 60° then ethanol was added and the solution stirred and heated at 60° overnight. The solution was evaporated and the residue poured into a ice-water mixture then basified with sat. aq. ammonium hydroxide. The mixture was then diluted with ethyl acetate (1 L), filtered to remove charred material, then the organic layer separated, dried and evaporated. The residue was purified by chromatography (silica gel, 40% ethyl acetate in hexanes) to give a mixture of the two isomeric title compounds as an orange oil (12.6 g; 53%). Ethyl 5-chloro-6-quinolinecarboxylate: 1H NMR (400 MHz, DMSO-d₆) □ 1.37 (t, J=7.07 Hz, 3 H) 4.42 (q, J=7.24 Hz, 2 H) 7.79 (dd, J=8.59, 4.29 Hz, 1 H) 8.04 (d, J=8.59 Hz, 1 H) 8.11 (d, J=8.84 Hz, 1 H) 8.74 (m, 1 H) 9.09 (dd, J=4.29, 1.52 Hz, 1 H). Ethyl 7-chloro-6-quinolinecarboxylate: 1H NMR (400 MHz, DMSO-d₆) δ ppm 1.37 (t, J=7.20 Hz, 3 H) 4.40 (q, J=7.07 Hz, 2 H) 7.66 (dd, J=8.34, 4.29 Hz, 1 H) 8.19 (s, 1 H) 8.53-8.58 (m, 2 H) 9.04 (dd, J=4.17, 1.64 Hz, 1 H).
b) (7-Chloro-6-quinolinyl)methanol and (5-chloro-6-quinolinyl)methanol. A solution of the compounds from Example 8a) (12.5 g; 0.053 mol.) in anhydrous THF (200 mL) was cooled to 5° C. then treated portionwise with lithium aluminum hydride (2.0 g; 0.053 mol.). The suspension was stirred at 5° C. for 1.5 h then quenched by the sequential addition of ethyl acetate (50.0 mL) and water (5.0 mL). The suspension was filtered and the filtrate dried and evaporated and the residue purified by chromatography (silica gel, gradient 10-50% ethyl acetate in hexanes) to afford a 3:1 mixture of (7-chloro-6-quinolinyl)methanol and (5-chloro-6-quinolinyl)methanol (0.67 g; 7%) as a yellow oil. Further elution afforded 1:5 mixture of (7-chloro-6-quinolinyl)methanol and (5-chloro-6-quinolinyl)methanol (3.3 g; 33%). (7-Chloro-6-quinolinyl)methanol: 1H NMR (400 MHz, DMSO-d₆) δ ppm 4.74 (d, J=5.05 Hz, 1 H) 5.69 (t, J=5.43 Hz, 1 H) 7.51 (dd, J=8.34, 4.29 Hz,1 H) 8.03 (s, 1 H) 8.10 (s, 1 H) 8.39 (d, J=8.08 Hz, 1 H) 8.88 (dd, J=4.17, 1.39 Hz, 1 H). 5-Chloro-6-quinolinyl)methanol: 1H NMR (400 MHz, DMSO-d₆) δ ppm 4.81 (d, J=5.81 Hz, 2 H) 5.65 (t, J=5.68 Hz, 1 H) 7.69 (dd, J=8.59, 4.04 Hz, 7 H) 7.99 (d, J=8.84 Hz, 1 H) 8.07 (d, J=8.59 Hz, 1 H) 8.61 (s, 1 H) 8.98 (dd, J=4.17, 1.64 Hz, 1 H)
c) 7-Chloro-6-quinolinecarbaldehyde. A solution of the compounds from Example 8b) (3:1 mixture of 7/5-isomers) (0.67 g; 3.5 mmol.) in acetonitrile (50.0 mL) was treated with manganese dioxide (1 g) and stirred at room temperature overnight. The black suspension was filtered through a pad of celite then evaporated to afford the title compound (0.454 g; 68%) as a grey powder. 1H NMR (400 MHz, DMSO-d₆) δ ppm 7.69 (dd, J=8.34, 4.04 Hz, 1 H) 8.22 (s, 1 H) 8.66 (s, 2 H) 9.08 (d, J=2.78 Hz, 1 H) 10.46 (s, 1 H).
d) (5Z)-5-[(7-Chloro-6-quinolinyl)methylidene]-2-[(2,6-dichlorophenyl)amino]-1,3-thiazol-4(5H)-one, trifluoroacetate salt. A suspension of 2-[(2,6-dichlorophenyl)amino]-1,3-thiazol-4(5H)-one (261 mg; 1.0 mmol.), the compound from Example 8c) (178 mg; 1.0 mmol.) and piperidine (0.12 mL) in ethanol (2.0 mL) was stirred and heated in a microwave reactor at 150° C. for 20 min. The mixture was cooled and filtered and the filtrate was evaporated and purified by chromatography (silica gel, gradient 10-50% ethyl acetate in hexanes) then further purified by chromatography (ODS silica gel, 10-100% acetonitrile in water (0.01% TFA)) to afford the title compound (16.8 mg; 4%) as a cream solid. 1H NMR (400 MHz, DMSO-d₆) δ ppm 7.22 (t, J=8.08 Hz, 1 H) 7.51-7.61 (m, 3 H) 8.00 (s, 1 H) 8.06 (s, 1 H) 8.24 (s, 1 H) 8.59 (d, J=8.08 Hz, 1 H) 8.96 (dd, J=4.04, 1.26 Hz, 1 H) 13.16 (s, 1 H).

### EXAMPLE 9

### (5Z)-5-[(8-Chloro-6-quinolinyl)methylidene]-2-[(2,6-dichlorophenyl)amino]-1,3-thiazol-4(5H)-one, trifluoroacetate salt

a) Ethyl 8-chloro-6-quinolinecarboxylate. A solution of 4-amino-3-chlorobenzoic acid (12.8 g; 0.069 mol.), glycerol (29.1 g; 0.316 mol.), sodium 3-nitrobenzenesulfonate (29.8 g; 0.132 mol.) and 75% aq. sulfuric acid (110 mL) was stirred and heated at 100° C. for 3 h then at 140° C. for 1 h. The mixture was allowed to cool to 60° then ethanol was added and the solution stirred and heated at 60° overnight. The solution was evaporated and the residue poured into a ice-water mixture then basified with sat. aq. ammonium hydroxide. The mixture was then diluted with ethyl acetate (1 L), filtered to remove charred material, then the organic layer separated, dried and evaporated. The residue was purified by chromatography (silica gel, 50% ethyl acetate in hexanes) to give the title compound (5.7 g; 35%) as a yellow solid. 1H NMR (400 MHz, DMSO-d₆) δ ppm 1.38 (t, J=7.07 Hz, 3 H) 4.40 (q, J=7.07 Hz, 2 H) 7.75 (dd, J=8.34, 4.29 Hz, 1 H) 8.27 (d, J=1.77 Hz, 1 H) 8.66 (d, J=1.77 Hz, 1 H) 8.68 (dd, J=8.46,1.64 Hz, 1 H) 9.13 (dd, J=4.04, 1.77 Hz, 1 H).
b) (8-Chloro-6-quinolinyl)methanol. A solution of the compounds from Example 9a) (4.7 g; 0.02 mol.) in anhydrous THF (100 mL) was cooled to 0° C. then treated portionwise with lithium aluminum hydride (0.76 g; 0.02 mol.). The suspension was stirred at 0° C. for 1 h then quenched by the sequential addition of ethyl acetate (35.0 mL) and water (2.0 mL). The suspension was filtered and the filtrate was dried and evaporated to afford the title compound (2.65 g; 68%) as a yellow solid. 1H NMR (400 MHz, DMSO-d₆) δ ppm 4.70 (s, 2 H) 5.27 (s, 1 H) 7.64 (dd, J=8.21, 4.17 Hz, 1 H) 7.85-7.94 (m, 2 H) 8.45 (dd, J=8.34, 1.77 Hz, 1 H) 8.98 (dd, J=4.29, 1.77 Hz, 1 H).
c) 8-Chloro-6-quinolinecarbaldehyde. A solution of the compounds from Example 9b) (2.5 g; 12.9 mmol.) in acetonitrile (100 mL) was treated with manganese dioxide (3.7 g) and stirred at room temperature overnight. The black suspension was filtered through a pad of celite then treated with further manganese dioxide (3.7 g) and stirred again overnight. The black suspension was filtered through a pad of celite then evaporated and the residue purified by chromatography (silica gel, 50% ethyl acetate in hexanes) to afford the title compound (1.2 g; 52%) as a colorless powder. 1H NMR (400 MHz, DMSO-d₆) δ ppm 7.81 (dd, J=8.21, 4.17 Hz, 1 H) 8.28 (d, J=1.77 Hz, 1 H) 8.68 (d, J=1.77 Hz, 1 H) 8.72 (dd, J=8.34, 1.77 Hz, 1 H) 9.18 (dd, J=4.17, 1.64 Hz, 1 H) 10.16 (s, 1 H). C₁₀H₆ClNO requires: % C, 62.68; % H, 3.16; % N, 7.31; found: % C, 62.40; % H, 2.90; % N, 7.00.
d) (5Z)-5-[(8-Chloro-6-quinolinyl)methylidene]-2-[(2,6-dichlorophenyl)amino]-1,3-thiazol-4(5H)-one, 0.75 trifluoroacetate salt. A suspension of 2-[(2,6-dichlorophenyl)amino]-1,3-thiazol-4(5H)-one (261 mg; 1.0 mmol.), the compound from Example 9c) (178.5 mg; 0.93 mmol.) and piperidine (0.11 mL) in ethanol (2.0 mL) was stirred and heated in a microwave reactor at 150° C. for 20 min. The mixture was cooled and directly purified by chromatography (ODS silica gel, 10-90% acetonitrile in water (0.01% TFA)) to afford the title compound (145 mg; 33%). 1H NMR (400 MHz, DMSO-d₆) δ ppm 7.25 (t, J=8.08 Hz, 1 H) 7.58 (d, J=8.08 Hz, 2 H) 7.67 (dd, J=8.08, 4.29 Hz, 1 H) 7.89 (s, 1 H) 8.06 (s, 1 H) 8.13 (d, J=1.26 Hz, 1 H) 8.62 (dd, J=8.34, 1.26 Hz, 1 H) 9.05 (dd, J=3.92, 1.39 Hz, 1 H) 13.08 (s, 1 H). C₁₉H₁₀Cl₃N₃OS.0.75 CF₃CO₂H requires: % C, 47.33; % H, 2.08; % N, 8.08; found: % C, 47.60; % H, 2.01; % N, 8.04.

### EXAMPLE 10

### (5Z)-2-[(2-Chlorophenyl)amino]-5-[(8-methyl-6-quinolinyl)methylidene]-1,3-thiazol-4(5H)-one

a) Ethyl 8-methyl-6-quinolinecarboxylate. A solution of 4-amino-3-methylbenzoic acid (10.0 g; 0.0606 mol.), glycerol (27.0 g; 0.12 mol.), sodium 3-nitrobenzenesulfonate (25.1 g; 0.272 mol.) and 75% aq. sulfuric acid (100 mL) was stirred and heated at 100° C. for 3 h then at 140° C. for 1 h. The mixture was allowed to cool to 60° then ethanol was added and the solution stirred and heated at 60° overnight. The solution was evaporated and the residue poured into a ice (500 g) then basified with sat. aq. ammonium hydroxide. The mixture was then diluted with dichloromethane (1 L), filtered to remove charred material, then the organic layer separated, dried and evaporated. The residue was purified by chromatography (silica gel, 20% ethyl acetate in hexanes) to give the title compound (7.7 g; 59%) as a cream solid. 1H NMR (400 MHz, DMSO-d₆) δ ppm 1.38 (t, J=7.07 Hz, 3 H) 2.76 (s, 3 H) 4.38 (q, J=7.07 Hz, 2 H) 7.64 (dd, J=8.21, 4.17 Hz, 1 H) 8.07 (dd, J=2.02, 1.01 Hz, 1 H) 8.52 (d, J=1.77 Hz, 1 H) 8.56 (dd, J=8.46, 1.89 Hz, 1 H) 9.04 (dd, J=4.17, 1.89 Hz, 1 H).
b) (8-Methyl-6-quinolinyl)methanol. A solution of the compounds from Example 10a) (7.0 g; 32.5 mmol.) in anhydrous THF (150 mL) was cooled to 5° C. then treated portionwise with lithium aluminum hydride (0.76 g; 0.02 mol.). The suspension was stirred at 5° C. for 1 h then quenched by the sequential addition of ethyl acetate (50.0 mL) and water (3.5 mL). The suspension was filtered and the filtrate was dried and evaporated and the residue triturated with a little diethyl ether to afford the title compound (3.7 g; 66%) as a colorless solid. 1H NMR (400 MHz, DMSO-d₆) δ ppm 2.72 (s, 3 H) 4.66 (d, J=5.56 Hz, 2 H) 5.39 (t, J=5.68 Hz, 1 H) 7.51 (dd, J=8.21, 4.17 Hz, 1 H) 7.57 (s, 1 H) 7.71 (s, 1 H) 8.31 (dd, J=8.08, 1.77 Hz, 1 H) 8.88 (dd, J=4.29, 1.77 Hz, 1 H).
c) 8-Methyl-6-quinolinecarbaldehyde. A solution of the compounds from Example 10b) (3.5 g; 0.020 mmol.) in acetonitrile (150 mL) was treated with manganese dioxide (5.0 g) and stirred at room temperature for 4h. The black suspension was filtered through a pad of celite then evaporated to afford the title compound (3.0 g; 88%) as a colorless powder. 1H NMR (400 MHz, DMSO-d₆) δ ppm 2.77 (s, 3 H) 7.68 (dd, J=8.21, 4.17 Hz, 1 H) 8.00 (d, J=1.01 Hz, 1 H) 8.49 (d, J=1.77 Hz, 1 H) 8.59 (dd, J=8.34, 1.77 Hz, 1 H) 9.08 (dd, J=4.17, 1.89 Hz, 1 H) 10.15 (s, 1 H).
d) (5Z)-2-[(2-Chlorophenyl)amino]-5-[(8-methyl-6-quinolinyl)methylidene]-1,3-thiazol-4(5H)-one, 0.75 acetate salt. A suspension of 2-[(2-chlorophenyl)amino]-1,3-thiazol-4(5H)-one (227 mg; 1.0 mmol.), the compound from Example 10c) (171 mg; 1.0 mmol.) and piperidine (0.109 mL) in ethanol (2.0 mL) was stirred and heated in a microwave reactor at 150° C. for 20 min. The mixture was cooled, poured into water and filtered to give a yellow solid which was crystallized from acetic acid to afford the title compound (37.3 mg; 9%). 1H NMR (400 MHz, DMSO-d₆) δ ppm 2.71 (s, 3 H) 7.19 (d, J=8.34 Hz, 1 H) 7.23 (td, J=7.58,1.52 Hz, 1 H) 7.39 (td, J=7.71, 1.26 Hz, 1 H) 7.56 (dt, J=8.15, 1.86 Hz, 2 H) 7.72 (s, 1 H) 7.80 (s, 1 H) 7.94 (d, J=1.26 Hz, 1 H) 8.44 (dd, J=8.34, 1.52 Hz, 1 H) 8.96 (dd, J=4.04, 1.77 Hz, 1 H) 12.75 (s, 1 H). C₂₀H₁₃Cl₂N₃OS. 0.75 C₂H₄O₂ requires: % C, 60.77; % H, 4.03; % N, 9.89; found; % C, 60.71; % H, 3.61; % N, 9.54.

### EXAMPLE 11

### (5Z)-5-[(5-Chloro-6-quinolinyl)methylidene]-2-[(2,6-dichlorophenyl)amino]-1,3-thiazol-4(5H)-one

a) 5-Chloro-6-quinolinecarbaldehyde. A solution of the compounds from Example 8b) (5:1 mixture of 5/7-isomers) (1.1 g; 5.7 mmol.) in acetonitrile (50.0 mL) was treated with manganese dioxide (1 g) and stirred at room temperature overnight. The black suspension was filtered through a pad of celite then evaporated to afford the title compound (0.85 g; 78%) as a colorless powder. 1H NMR (400 MHz, DMSO-d₆) δ ppm 7.81 (dd, J=8.59, 4.29 Hz, 1 H) 8.10 (s, 2 H) 8.80 (dd, J=8.59, 1.52 Hz, 1 H) 9.13 (dd, J=4.29, 1.52 Hz, 1 H) 10.59 (s, 1 H).
b) (5Z)-5-[(5-Chloro-6-quinolinyl)methylidene]-2-[(2,6-dichlorophenyl)amino]-1,3-thiazol-4(5H)-one. A suspension of 2-[(2,6-dichlorophenyl)amino]-1,3-thiazol-4(5H)-one (261 mg; 1.0 mmol.), the compound from Example 11a) (171 mg; 1.0 mmol.) and piperidine (0.12 mL) in ethanol (2.0 mL) was stirred and heated in a microwave reactor at 150° C. for 20 min. The mixture was cooled and filtered to give a yellow solid which was treated with warm acetic acid (5.0 mL) with sonication and filtered to afford the title compound (40.2 mg; 9%). 1H NMR (400 MHz, DMSO-d₆) δ ppm 7.23 (t, J=8.21 Hz, 1 H) 7.57 (d, J=8.34 Hz, 2 H) 7.69-7.88 (m, 2 H) 8.00-8.24 (m, 2 H) 8.69 (d, J=8.08 Hz, 1 H) 9.04 (dd, J=4.17, 1.39 Hz, 1 H) 13.18 (s, 1 H). C₁₉H₁₀Cl₃N₃OS requires: % C, 52.49; % H, 2.32; % N, 9.67; found: % C, 52.20; % H, 2.12; % N, 9.55.

### EXAMPLE 12

### (5Z)-2-[(2,6-Dichlorophenyl)amino]-5-[(2-pentyl-6-quinolinyl)methylidene]-1,3-thiazol-4(5H)-one

The title compound was made by following the method in Example 6f) as a yellow solid (68%). 2-pentyl-6-quinolinecarbaldehyde was used in place of 2-chloro-6-quinolinecarbaldehyde. 1H NMR (400 MHz, DMSO-d₆) δ ppm 13.00 (s, 1 H) 8.35 (d, J=8.59 Hz, 1 H) 8.09 (d, J=1.26 Hz, 1 H) 7.99 (d, J=8.59 Hz, 1 H) 7.88 (s, 1 H) 7.77 (dd, J=8.84, 1.77 Hz, 1 H) 7.58 (d, J=8.08 Hz, 2 H) 7.46 (d, J=8.59 Hz, 1 H) 7.24 (t, J=8.08 Hz, 1 H) 2.90 (t, J=8.00 Hz, 2 H) 1.68-1.81 (m, 2 H) 1.24-1.38 (m, 4 H) 0.81-0.90 (m, 3 H)

### EXAMPLE 13

### 6-{(Z)-[2-[(2,6-Dichlorophenyl)amino]-4-oxo-1,3-thiazol-5(4H)-ylidene]methyl}-2(1H)-quinolinone

a) 2-Oxo-1,2-dihydro-6-quinolinecarbaldehyde. The title compound was made by following the method described in Example 6d) as yellow oil (77%). The compound from Example 6b) was used in place of the compound from Example 6c). MS(ES+) m/e 174 [M+H]⁺.
b) 6-((Z)-{2-[(2,6-Dichlorophenyl)methylidene]-4-oxo-1,3-thiazolidin-5-ylidene}methyl)-2(1H)-quinolinone. The title compound was made by following the procedure described in Example 6f) as a yellow solid (40%). The compound from Example 13a) was used in place of the compound Example 6d). 1H NMR (400 MHz, DMSO-d₆) δ ppm 11.86 (s, 1 H) 7.85-8.06 (m, 1 H) 7.51-7.82 (m, J=8.72, 2.40 Hz, 2 H) 7.35 (s, 3 H) 7.21-7.30 (m, 1 H) 6.98 (s, 1 H) 6.50 (s, 1 H)

### EXAMPLE 14

### (5Z)-2-[(2,6-Dichlorophenyl)amino]-5-[(2-ethyl-6-quinolinyl)methylidene]-1,3-thiazol-4(5H)-one

a) 2-Ethyl-6-quinolinecarbaldehyde. The title compound was made by following the method described in Example 6d) as a brown solid (31%). 6-bromo-2-ethylquinoline was used in place of the compound from Example 6c). 1H NMR (400 MHz, CHLOROFORM-d) δ ppm 10.22 (s, 1 H) 8.38 (d, J=1.77 Hz, 1 H) 8.30 (d, J=8.59 Hz, 1 H) 8.22-8.27 (m, 1 H) 8.11-8.19 (m, 1 H) 7.54 (d, J=8.59 Hz, 1 H) 4.14 (q, J=7.07 Hz, 2 H) 1.28 (t, J=7.07 Hz, 3 H).
b) (5Z-2-[(2,6-Dichlorophenyl)amino]-5-[(2-ethyl-6-quinolinyl)methylidene]-1,3-thiazol-4(5H)-one. The title compound was made by following the procedure described in Example 6f) as a yellow solid (43%). The compound from Example 14a) was used in place of the compound from Example 6d). 1H NMR (400 MHz, DMSO-d₆) δ ppm 13.02 (s, 1 H) 8.85 (d, J=2.02 Hz, 1 H) 8.26 (s, 1 H) 8.01-8.11 (m, 2 H) 7.90 (s, 1 H) 7.78 (dd, J=8.72, 1.89 Hz, 1 H) 7.59 (d, J=8.08 Hz, 2 H) 7.25 (t, J=8.08 Hz, 1 H) 2.81 (q, J=7.58 Hz, 2 H) 1.28 (t, J=7.58 Hz, 3 H)

### EXAMPLE 15

### (5Z)-2-[(2,6-Dichlorophenyl)amino]-5-{[2-(methyloxy)-6-quinolinyl]methylidene}-1,3-thiazol-4(5H)-one

a) 6-Bromo-2-(methyloxy)quinoline. To a solution of Example 6c) (100 mg, 0.82 mmol) in dry MeOH (2 mL) was added sodium methoxide (25% in methanol, 187 μL, 0.82 mmol). The mixture was heated to 100° C. for 30 minutes in a Biotage Initiator microwave synthesizer, then cooled to the ambient temperature and quenched with ice water. The precipitate was filtered, washed with water and dried in vacuo to afford the title compound as a white solid (30 mg, 31%) MS(ES+) m/e 239 [M+H]⁺.
b) 2-(Methyloxy)-6-quinolinecarbaldehyde. The title compound was made by following the procedure in Example 6d) as a yellow solid (30%). The compound from Example 15a) was used in place of the compound from Example 6c). MS(ES+) m/e 188 [M+H]⁺.
c) (5Z)-2-[(2,6-Dichlorophenyl)amino]-5-{[2-(methyloxy)-6-quinolinyl]methylidene}-1,3-thiazol-4(5H)-one. The title compound was made by following the method in Example 6f) as a yellow solid (12%). The compound from Example 15b) was used in place of the compound from Example 6d). 1H NMR (400 MHz, CHLOROFORM-d) δ ppm 8.10 (d, J=9.09 Hz, 1 H) 7.93-7.99 (m, 2 H) 7.83 (d, J=1.52 Hz, 1 H) 7.69 (dd, J=8.72, 1.89 Hz, 1 H) 7.47 (d, J=8.08 Hz, 2 H) 7.22 (t, J=8.21 Hz, 1 H) 7.01 (d, J=9.09 Hz, 1 H) 4.16 (s, 3 H)

### EXAMPLE 16

### (5Z)-2-[(2,6-Dichlorophenyl)amino]-5-{[2-(dimethylamino)-6-quinolinyl]methylidene}-1,3-thiazol-4(5H)-one

The title compound was made by following the method described in Example 6f) as a yellow solid (48%). Dimethylamine was used in place of piperidine. 1H NMR (400 MHz, DMSO-d₆) δ ppm 8.39 (d, J=9.60 Hz, 1 H) 8.01 (s, 1 H) 7.95 (br. s., 1 H) 7.82 (s, 1 H) 7.80 (s, 1 H) 7.58 (d, J=8.08 Hz, 2 H) 7.39 (d, J=9.35 Hz, 1 H) 7.24 (t, J=8.08 Hz, 1 H) 3.35 (s, 6 H)

### EXAMPLE 17

### (5Z)-2-[(2,6-Dichlorophenyl)amino]-5-[(4-hydroxy-6-quinolinyl)methylidene]-1,3-thiazol-4(5H)-one

a) Ethyl 6-bromo-4-hydroxy-3-quinolinecarboxylate. The mixture of 4-bromoaniline (5.0 g, 29.6 mmol) and diethyl propanedioate-(ethyloxy)ethene (5.8 mL, 29.6 mmol) in phenyl ether (50 mL) was refluxed overnight. The product was cooled, then petroleum ether (200 mL) was added. The precipitate was filtered, washed with petroleum ether and dried in vacuo to afford the title compound as a grey solid (5.3 g, 65%). 1H NMR (400 MHz, DMSO-d₆) δ ppm 10.66 (d, J=1 3.89 Hz, 1 H) 8.36 (d, J=13.64 Hz, 1 H) 7.56 (d, J=8.84 Hz, 1 H) 7.37 (d, J=9.09 Hz, 1 H) 4.13 (q, J=7.07 Hz, 1 H) 1.24 (t, J=7.07 Hz, 3 H).
b) 6-Bromo-4-quinolinol. The mixture of the compound from Example 17a) (2 g, 6.8 mmol) in NaOH ( 6 N, 100 mL) was refluxed overnight, cooled and filtered. The filtrate was acidified. The precipitate was collected and refluxed in phenyl ether for two hours. The product was cooled, then petroleum ether was added until precipitate appeared. The precipitate was collected, washed with petroleum ether and dried over vacuo to afford the title compound as a white solid (1.2 g, 85%). 1H NMR (400 MHz, DMSO-d₆) δ ppm 12.06 (br. s.,1 H) 8.16 (d, J=2.27 Hz, 1 H) 7.90-8.02 (m, 1 H) 7.79 (dd, J=8.84, 2.53 Hz, 1 H) 7.55 (d, J=8.84 Hz, 1 H) 6.08 (d, J=7.33 Hz, 1 H).
c) 4-Hydroxy-6-quinolinecarbaldehyde. Sodium hydride (60% dispersion in mineral oil, 36 mg, 0.89 mmol) was added to the solution of the compound from Example 17b) (200 mg, 0.89 mmol) in THF (5 mL) at 0° C. The mixture was kept stirring at 0° C. for half hour and then cooled to −78° C. N-butyl lithium (1.6 M in hexane, 0.55 mL, 0.89 mmol) was added dropwise. After half hour, DMF (69 μL, 0.89 mmol) was added. The product was let to raise the ambient temperature, quenched by saturated NH₄Cl solution and extracted with ethyl acetate. The extract was dried over magnesium sulfate, concentrated in vacuo and purified via flash column chromatography (0-100% ethyl acetate in hexanes) to give the title compound as a white solid (40 mg, 28%). MS(ES+) m/e 174 [M+H]⁺.
d) (5Z)-2-[(2,6-Dichlorophenyl)amino]-5-[(4-hydroxy-6-quinolinyl)methylidene]-1,3-thiazol-4(5H)-one. The title compound was made by following the method described in Example 6f) as yellow solid (10%). The compound from Example 17c) was used in place of the compound from Example 6d). 1H NMR (400 MHz, DMSO-d₆) δ ppm 8.12 (d, J=1.77 Hz, 1 H) 7.98 (d, J=7.33 Hz, 1 H) 7.77-7.87 (m, 2 H) 7.66 (d, J=8.84 Hz, 1 H) 7.58 (d, J=8.08 Hz, 2 H) 7.25 (t, J=8.08 Hz, 1 H) 6.14 (d, J=7.33 Hz, 1 H)

### EXAMPLE 18

### (5Z)-2-[(2,6-Dichlorophenyl)amino]-5-[(3-methyl-6-quinolinyl)methylidene]-1,3-thiazol-4(5H)-one

a) 6-Bromo-3-methylquinoline. The title compound was made by following the procedure described in Example 7a) as a yellow solid (23%). 2-methyl-2-propenal was used in place of (2E)-2-butenal. 1H NMR (400 MHz, CHLOROFORM-d) δ ppm 8.79 (d, J=2.02 Hz, 1 H) 7.98 (d, J=8.84 Hz, 1 H) 7.93 (d, J=2.02 Hz, 1 H) 7.86 (s, 1 H) 7.73 (dd, J=8.97, 2.15 Hz, 1 H) 2.55 (s, 3 H)
b) 3-Methyl-6-quinolinecarbaldehyde. The title compound was made by following the procedure described in Example 6d) as a white solid (68%). The compound from Example 18a) was used in place of the compound from Example 6c). MS(ES+) m/e 172 [M+H]⁺.
c) (5Z)-2-[(2,6-Dichlorophenyl)amino]-5-[(3-methyl-6-quinolinyl)methylidene]-1,3-thiazol-4(5H)-one. The title compound was made by following the procedure described in Example 6f) as a yellow solid (10%). The compound from Example 18b) was used in place of the compound from Example 6d). 1H NMR (400 MHz, DMSO-d₆) δ ppm 13.01 (s, 1 H) 8.80 (d, J=1.77 Hz, 1 H) 8.21 (s, 1 H) 8.03 (dd, J=4.80, 3.79 Hz, 2 H) 7.89 (s, 1 H) 7.70-7.80 (m, 1 H) 7.54-7.62 (m, 2 H) 7.24 (t, J=8.21 Hz, 1 H) 2.46 (s, 3 H)

### EXAMPLE 19

### (5Z)-2-[(2,6-Dichlorophenyl)amino]-5-{[3-(1-methylethyl)-6-quinolinyl]methylidene}-1,3-thiazol-4(5H)-one

a) 3-(1-Methylethyl)-6-quinolinecarbaldehyde. The title compound was made by following the procedure described in Example 6d) as a white solid (50%). 6-(bromomethyl)-3-(1-methylethyl)quinoline was used in place of the compound from Example 6c). MS(ES+) m/e 200 [M+H]⁺.
b) (5Z)-2-[(2,6-Dichlorophenyl)amino]-5-{[3-(1-methylethyl)-6-quinolinyl]methylidene}-1,3-thiazol-4(5H)-one. The title compound was made by following the procedure described in Example 24 as a yellow solid (39%). The compound from Example 19a) was used in place of the compound from Example 22d). 1H NMR (400 MHz, DMSO-d₆) δ ppm 13.02 (br. s., 1 H) 8.89 (d, J=2.02 Hz, 1 H) 8.26 (d, J=1.52 Hz, 1 H) 7.97-8.11 (m, 2 H) 7.88 (s, 1 H) 7.77 (dd, J=8.84, 1.77 Hz, 1 H) 7.58 (d, J=8.08 Hz, 2 H) 7.24 (t, J=8.08 Hz, 1 H) 3.03-3.23 (m, 1 H) 1.31 (d, J=7.07 Hz, 6 H)

### EXAMPLE 20

### (5Z)-2-[(2,6-Dichlorophenyl)amino]-5-{[4-(methylamino)-6-quinolinyl]methylidene}-1,3-thiazol-4(5H)-one

a) 6-Bromo-N-methyl-4-quinolinamine. The mixture of the compound from Example 22a) (100 mg, 0.41 mmol) and methylamine (2.0 M in methanol, 2 mL, 4 mmol) was heated to 120° C. for 3 hours in a Biotage Initiator microwave synthesizer. The product was cooled, concentrated and purified via flash chromatography (0-10% methanol in methylene chloride) to afford the title compound as a white solid (40 mg, 41%). 1H NMR (400 MHz, CHLOROFORM-d) δ ppm 8.39 (d, J=5.81 Hz, 1 H) 8.04 (d, J=1.77 Hz, 1 H) 7.80 (d, J=9.09 Hz, 1 H) 7.67 (dd, J=8.84, 2.02 Hz, 1 H) 6.37 (d, J=5.81 Hz, 1 H) 3.02 (s, 3 H).
b) 4-(Methylamino)-6-quinolinecarbaldehyde. The title compound was made by following the procedure described in Example 6d) as a white solid (66%). The compound from Example 20a) was used in place of 6-bromo-4-chloro quinoline. MS(ES+) m/e 187 [M+H]⁺.
c) (5Z)-2-[(2,6-Dichlorophenyl)amino]-5-{[4-(methylamino)-6-quinolinyl]methylidene}-1,3-thiazol-4(5H)-one. The title compound was made following the procedure described in Example 24 as a yellow solid (41%). The compound from Example 20a) was used in place of the compound from Example 22). 1H NMR (400 MHz, DMSO-d₆) δ ppm 8.37-8.56 (m, 2 H) 7.97 (br s,1 H) 7.86 (d, J=8.84 Hz, 1 H) 7.64-7.72 (m, 2 H) 7.55 (d, J=8.08 Hz, 2 H) 7.20 (t, J=7.71 Hz, 1 H) 6.53 (d, J=4.80 Hz, 1 H) 2.96 (s, 3 H)

### EXAMPLE 21

### Ethyl 4-chloro-6-{(Z)-[2-[(2,6-dichlorophenyl)amino]-4-oxo-1,3-thiazol-5(4H)-ylidene]methyl}-3-quinolinecarboxylate

a) Ethyl 6-bromo-4-chloro-3-quinolinecarboxylate. The mixture of the compound from Example 17a) (2.0 g, 6.8 mmol) in POCl₃ (20 mL) was refluxed overnight. The product was cooled, quenched with ice water, diluted with ethyl acetate, washed with NaHCO₃ and brine. The organic layer was separated, dried over magnesium sulfate, filtered, concentrated under vacuo and purified via flash chromatography (0-100% ethyl acetate in hexane) to afford the title compound as a white solid (1.5 g, 72%). 1H NMR (400 MHz, CHLOROFORM-d) δ ppm 9.22 (s, 1 H) 8.59 (d, J=2.02 Hz, 1 H) 8.03 (d, J=8.84 Hz, 1 H) 7.93 (dd, J=8.97, 2.15 Hz, 1 H) 4.53 (q, J=7.24 Hz, 2 H) 1.49 (t, J=7.20 Hz, 3 H).
b) Ethyl 4-chloro-6-ethenyl-3-quinolinecarboxylate. The mixture of the compound from Example 21a) (500 mg, 1.6 mmol), K₂CO₃ (219 mg, 1.6 mmol), tetrakis(triphenylphosphine)palladium (184 mg, 0.16 mmol) and triethenyl borate pyridine (191 mg, 0.8 mmol in 10 mL of ethylene glycol dimethyl ether/water=3/1 was heated at 85° C. for an hour. The product was cooled and quenched by water, extracted by ethyl acetate. The extract was dried over MgSO₄, filtered and purified via flash chromatography (0-100% ethyl acetate in hexane) to afford the title compound as a white solid (281 mg, 68%). 1H NMR (400 MHz, CHLOROFORM-d) δ ppm 8.29 (d, J=1.77 Hz, 1 H) 8.10 (d, J=8.59 Hz, 1 H) 8.00 (dd, J=8.72, 1.89 Hz, 1 H) 6.96 (dd, J=17.68, 10.86 Hz, 1 H) 6.00 (d, J=17.68 Hz, 1 H) 5.51 (d, J=10.86 Hz, 1 H) 4.52 (q, J=7.24 Hz, 2 H) 1.48 (t, J=7.07 Hz, 3 H).
c) Ethyl 4-chloro-6-formyl-3-quinolinecarboxylate. The title compound was made by following the procedure described in Example 22c) as a yellow solid (80%). Example 21a) was used in place of Example 22b). 1H NMR (400 MHz, CHLOROFORM-d) δ ppm 10.21 (s, 1 H) 9.25 (s, 1 H) 8.82 (d, J=1.26 Hz, 1 H) 8.24 (dd, J=8.59, 1.77 Hz, 1 H) 8.17 (d, J=8.59 Hz, 1 H) 4.49 (q, J=7.24 Hz, 2 H) 1.45 (t, J=7.07 Hz, 3 H).
d) Ethyl 4-chloro-6-{(Z)-[2-[(2,6-dichlorophenyl)amino]-4-oxo-1,3-thiazol-5(4H)-ylidene]methyl}-3-quinolinecarboxylate. The title compound was made by following the procedure described in Example 22d) as a yellow solid (80%). Example 21b) was used in place of Example 22c). 1H NMR (400 MHz, DMSO-d₆) δ ppm 13.06 (br. s., 1 H) 9.16 (s, 1 H) 8.52 (s, 1 H) 8.20 (d, J=8.84 Hz, 1 H) 7.92-8.05 (m, 2 H) 7.57 (dd, J=7.83, 3.03 Hz, 2 H) 7.17-7.29 (m, 1 H) 4.43 (q, J=7.07 Hz, 2 H) 1.38 (t, J=7.07 Hz, 3 H)

### EXAMPLE 22

### (5Z)-5-[(4-Chloro-6-quinolinyl)methylidene]-2-[(2,6-dichlorophenyl)amino]-1,3-thiazol-4(5H)-one

a) 6-Bromo-4-chloroquinoline. The solution of the compound from Example 17b (2 g, 8.9 mmol) in POCl₃ (10 mL) was refluxed overnight, cooled and quenched by ice water. The precipitate was collected, washed with H₂O and dried in vacuo to afford the title compound as a grey solid (1.8 g, 84%). 1H NMR (400 MHz, DMSO-d₆) δ ppm 8.90 (d, J=4.80 Hz, 1 H) 8.37 (d, J=2.02 Hz, 1 H) 8.07 (d, J=8.59 Hz, 1 H) 8.03 (dd, J=8.84, 2.02 Hz, 1 H) 7.86 (d, J=4.55 Hz, 1 H).
b) 4-Chloro-6-ethenylquinoline. The mixture of the compound from Example 22a) (4.0 g, 16.5 mmol), tetrakis(triphenylphosphine)palladium (190 mg, 0.16 mmol) and tributyl(vinyl)tin (4.8 mL, 16.5 mmol) in dioxane (10 mL) was heated to 150° C. for 40 minutes in a Biotage Initiator microwave synthesizer. The product was concentrated under vacuo and purified via flash chromatography (0-10% methanol in methylene chloride) to afford the title compound as a yellow solid (2.5 g, 80%). 1H NMR (400 MHz, CHLOROFORM-d) δ ppm 8.74 (d, J=4.80 Hz, 1 H) 8.13 (d, J=2.02 Hz, 1 H) 8.10 (d, J=8.84 Hz, 1 H) 7.94 (dd, J=8.84, 2.02 Hz, 1 H) 7.49 (d, J=4.55 Hz, 1 H) 6.95 (dd, J=17.68, 10.86 Hz, 1 H) 5.98 (d, J=17.43 Hz, 1 H) 5.48 (d, J=10.86 Hz, 1 H).
c) 4-Chloro-6-quinolinecarbaldehyde. Sodium periodate (3.77 g, 17.6 mmo) was added to the solution of the compound from Example 22b) (835 mg, 4.4 mmol), 2,6-dimethylpyridine (1 mL, 8.8 mmol) and osmium tetroxide (2.5% in t-butanol, 1.1 mL, 0.088 mmo) in dioxane/water=3:1 (20 mL). The product was kept stirring for an hour at ambient temperature, quenched with water and extracted with ethyl acetate. The extract was dried over MgSO₄, filtered, concentrated under vacuo and purified via flash chromatography (0-100% ethyl acetate in hexane) to afford the title compound as a white solid (710 mg, 85%). 1H NMR (400 MHz, CHLOROFORM-d) δ ppm 10.28 (s, 1 H) 8.95 (d, J=4.55 Hz, 1 H) 8.78 (s, 1 H) 8.29 (dd, J=8.84, 1.52 Hz, 1 H) 8.26 (d, J=8.84 Hz, 1 H) 7.64 (d, J=4.80 Hz, 1 H).
d) (5Z)-5-[(4-Chloro-6-quinolinyl)methylidene]-2-[(2,6-dichlorophenyl)amino]-1,3-thiazol-4(5H)-one. The title compound was made by following the procedure described in Example 6f) as a yellow solid (32%). The compound from Example 22c) was used in place of the compound from Example 6d). Sodium acetate was used in place of piperidine. 1H NMR (400 MHz, DMSO-d₆) δ ppm 13.08 (s,1 H) 8.88 (d, J=4.80 Hz, 1 H) 8.39 (d, J=1.52 Hz, 1 H) 8.18 (d, J=8.59 Hz, 1 H) 8.03 (s, 1 H) 7.89-7.96 (m, 1 H) 7.83 (d, J=4.80 Hz, 1 H) 7.59 (d, J=8.08 Hz, 2 H) 7.25 (t, J=8.08 Hz, 1 H)

### EXAMPLE 23

### (5Z)-2-[(2,6-Dichlorophenyl)amino]-5-{[4-(methyloxy)-6-quinolinyl]methylidene}-1,3-thiazol-4(5H)-one

The title compound was made by following the method described in Example 24 as a yellow solid (22%). K₂CO₃ is used in place of piperidine. 1H NMR (400 MHz, DMSO-d₆) δ ppm 13.01 (br. s., 1 H) 8.78 (d, J=5.05 Hz, 1 H) 8.29 (s, 1 H) 8.02 (d, J=8.84 Hz, 1 H) 7.93 (s, 1 H) 7.72-7.85 (m, 1 H) 7.59 (d, J=8.34 Hz, 2 H) 7.25 (t, J=8.08 Hz, 1 H) 7.09 (d, J=5.30 Hz, 1 H) 4.03 (s, 3 H)

### EXAMPLE 24

### (5Z)-2-[(2,6-Dichlorophenyl)amino]-5-{[4-(1-piperidinyl)-6-quinolinyl]methylidene}-1,3-thiazol-4(5H)-one trifluoroacetate

The mixture of the compound from Example 22d) (20 mg, 0.046 mmol) and piperidine (46 μL, 0.46 mmol) in methanol (2 mL) was heated to 150° C. for 30 minutes in a Biotage Initiator microwave synthesizer. The product was concentrated and purified via preparative HPLC (YMC 75×30 mm column, 0.1% TFA in water and 0.1% TFA in acetonitrile) to afford the title compound as a yellow solid (11 mg, 49%). 1H NMR (400 MHz, DMSO-d₆) δ ppm 8.64 (d, J=7.07 Hz, 1 H) 8.08-8.13 (m, 1 H) 7.99-8.05 (m, 2 H) 7.94 (s,1 H) 7.60 (d, J=8.08 Hz, 2 H) 7.27 (t, J=8.21 Hz, 1 H) 7.20 (d, J=6.82 Hz, 1 H) 3.65 (br. s., 4 H) 1.55-1.66 (m, 6 H)

### EXAMPLE 25

### (5Z)-2-[(2,6-Dichlorophenyl)amino]-5-{[4-(4-morpholinyl)-6-quinolinyl]methylidene}-1,3-thiazol-4(5H)-one

The title compound was made by following the procedure described in Example 24 as a yellow solid (50%). Morphine is used in place of piperidine. 1H NMR (400 MHz, DMSO-d₆) δ ppm 13.06 (br. s., 1 H) 8.74 (d, J=4.80 Hz, 1 H) 8.06 (s, 1 H) 8.01 (d, J=8.84 Hz, 1 H) 7.87-7.96 (m, 2 H) 7.59 (d, J=8.08 Hz, 2 H) 7.26 (t, J=8.21 Hz, 1 H) 7.04 (d, J=4.80 Hz, 1 H) 3.58 (s, 4 H) 3.09 (s, 4 H)

### EXAMPLE 26

### (5Z)-2-[(2,6-Dichlorophenyl)amino]-5-[(8-fluoro-6-quinolinyl)methylidene]-1,3-thiazol-4(5H)-one

a) 5-Chloro-6-quinolinecarbaldehyde. A solution of the compounds from Example 8b) (5:1 mixture of 5/7-isomers) (1.1 g; 5.7 mmol.) in acetonitrile (50.0 mL) was treated with manganese dioxide (1 g) and stirred at room temperature overnight. The black suspension was filtered through a pad of celite then evaporated to afford the title compound (0.85 g; 78%) as a colorless powder. 1H NMR (400 MHz, DMSO-d₆) δ ppm 7.81 (dd, J=8.59, 4.29 Hz, 1 H) 8.10 (s, 2 H) 8.80 (dd, J=8.59, 1.52 Hz, 1 H) 9.13 (dd, J=4.29, 1.52 Hz, 1 H) 10.59 (s, 1 H).
b) (5Z)-5-[(5-Chloro-6-quinolinyl)methylidene]-2-[(2,6-dichlorophenyl)amino]-1,3-thiazol-4(5H)-one. A suspension of 2-[(2,6-dichlorophenyl)amino]-1,3-thiazol-4(5H)-one (261 mg; 1.0 mmol.), the compound from Example 11a) (171 mg; 1.0 mmol.) and piperidine (0.12 mL) in ethanol (2.0 mL) was stirred and heated in a microwave reactor at 150° C. for 20 min. The mixture was cooled and filtered to give a yellow solid which was treated with warm acetic acid (5.0 mL) with sonication and filtered to afford the title compound (40.2 mg; 9%). 1H NMR (400 MHz, DMSO-d₆) δ ppm 7.23 (t, J=8.21 Hz, 1 H) 7.57 (d, J=8.34 Hz, 2 H) 7.69-7.88 (m, 2 H) 8.00-8.24 (m, 2 H) 8.69 (d, J=8.08 Hz, 1 H) 9.04 (dd, J=4.17, 1.39 Hz, 1 H) 13.18 (s, 1 H). C₁₉H₁₀Cl₃N₃OS requires: % C, 52.49; % H, 2.32; % N, 9.67; found: % C, 52.20; % H, 2.12; % N, 9.55.

### EXAMPLE 27

### (5Z)-2-[(2,6-Dichlorophenyl)amino]-5-({4-[(3,3-dimethylbutyl)amino]-6-quinolinyl}methylidene)-1,3-thiazol-4(5H)-one trifluoroacetate

The title compound was made by following the procedure described in Example 24 as a yellow solid (44%). 3,3-dimethyl-1-butanamine is used in place of piperidine. 1H NMR (400 MHz, DMSO-d₆) δ ppm 13.90 (br. s., 1 H) 13.12 (br. s., 1 H) 9.13 (s, 1 H) 8.74 (s, 1 H) 8.54 (d, J=7.07 Hz, 1 H) 7.95 (q, J=9.01 Hz, 2 H) 7.77 (s, 1 H) 7.58 (d, J=8.08 Hz, 2 H) 7.25 (t, J=8.08 Hz, 1 H) 6.89 (d, J=7.07 Hz, 1 H) 3.48-3.59 (m,2 H) 1.52-1.69 (m, 2 H) 0.97 (s, 9 H)

### EXAMPLE 28

### Ethyl 6-{(Z)-[2-[(2,6-dichlorophenyl)amino]-4-oxo-1,3-thiazol-5(4H)-ylidene]methyl}-3-quinolinecarboxylate

a) Ethyl 6-formyl-3-quinolinecarboxylate. The mixture of the compound from Example 22b) (800 mg, 3.0 mmol), triethylamine (0.64 mL, 4.5 mmol) and palladium on carbon (10% weight, 80mg) in 16 mL of solvent (MeOH/THF=3/1) was conducted hydrogenation with hydrogen in balloon under ambient temperature for 2 hours. The product was filtered, concentrated and purified via flash chromatography (0-100% ethyl acetate in hexane) to afford the title compound as a white solid (450 mg, 65%). MS(ES+) m/e 230 [M+H]⁺.
b) Ethyl 6-{(Z)-[2-[(2,6-dichlorophenyl)amino]-4-oxo-1,3-thiazol-5(4H)-ylidene]methyl}-3-quinolinecarboxylate. The title compound was made by following the procedure described in Example 24 as a yellow solid (35%). The compound from Example 28a) was used in place of the compound from Example 22d). 1H NMR (400 MHz, DMSO-d₆) δ ppm 13.06 (br. s., 1 H) 9.33 (d, J=2.02 Hz, 1 H) 9.13 (d, J=1.52 Hz, 1 H) 8.37 (s, 1 H) 8.17 (d, J=8.84 Hz, 1 H) 8.00 (dd, J=8.97, 1.64 Hz, 1 H) 7.90 (s, 1 H) 7.58 (d, J=8.08 Hz, 2 H) 7.25 (t, J=8.08 Hz, 1 H) 4.42 (q, J=7.24 Hz, 2 H) 1.38 (t, J=7.07 Hz, 3 H)

### EXAMPLE 29

### 6-{(Z)-[2-[(2,6-Dichlorophenyl)amino]-4-oxo-1,3-thiazol-5(4H)-ylidene]methyl}-3-quinolinecarboxylic acid

The mixture of the compound from Example 28b) (20 mg, 0.04 mmol), NaOH (6N, 2 mL) and MeOH (2 mL) was kept stirring overnight. The product was acidified. The precipitate was collected, washed with water and dried under vacuo to afford the title compound as a yellow solid (17 mg, 90%). 1H NMR (400 MHz, DMSO-d₆) δ ppm 13.65 (br. s., 1 H) 13.09 (s, 1 H) 9.31 (d, J=1.77 Hz, 1 H) 9.09 (s, 1 H) 8.37 (s, 1 H) 8.15 (d, J=8.59 Hz, 1 H) 7.97 (dd, J=8.97, 1.64 Hz, 1 H) 7.90 (s, 1 H) 7.58 (d, J=8.08 Hz, 2 H) 7.24 (t, J=8.08 Hz, 1 H)

### EXAMPLE 30

### 6-{(Z)-[2-[(2,6-Dichlorophenyl)amino]-4-oxo-1,3-thiazol-5(4H)-ylidene]methyl}-3-quinolinecarboxamide

Ammonium hydroxide (1 mL) was added to the solution of the compound from Example 28 (20 mg, 0.042 mmol) in methanol (1 mL). The mixture was kept stirring overnight, concentrated under vacuo and acidified. The precipitate was collected, washed with water and dried under vacuo to afford the title compound as a yellow solid (20 mg, 98%). 1H NMR (400 MHz, DMSO-d₆) δ ppm 9.30 (d, J=2.02 Hz, 1 H) 8.91 (d, J=1.52 Hz, 1 H) 8.19 (s, 1 H) 8.13 (d, J=8.59 Hz, 1 H) 7.87-7.99 (m, 2 H) 7.58 (d, J=8.08 Hz, 2 H) 7.24 (t, J=7.96 Hz, 1 H)

### EXAMPLE 31

### (5Z)-5-({4-[(2-Cyclopropylethyl)amino]-6-quinolinyl}methylidene)-2-[(2,6-dichlorophenyl)amino]-1,3-thiazol-4(5H)-one trifluoroacetate

The mixture of the compound from Example 22d) (48 mg, 0.11 mmol), N,N-diisopropylethylamine (58 μL, 0.33 mmol) and 2-cyclopropylethanamine hydrochloride (38 mg, 0.33 mmol) in methanol (5 mL) was heated to 150° C. for an hour in a Biotage Initiator microwave synthesizer. The product was concentrated and purified via preparative HPLC (YMC 75×30 mm column, 0.1% TFA in water and 0.1% TFA in acetonitrile ) to afford the title compound as a yellow solid (12 mg, 43%). 1H NMR (400 MHz, DMSO-d₆) δ ppm 13.96 (br. s., 1 H) 13.12 (br. s., 1 H) 9.16-9.35 (m, 1 H) 8.75 (s, 1 H) 8.52 (d, J=7.07 Hz, 1 H) 7.88-8.05 (m, 2 H) 7.77 (s, 1 H) 7.57 (d, J=8.08 Hz, 2 H) 7.24 (t, J=8.08 Hz, 1 H) 6.94 (d, J=7.07 Hz, 1 H) 3.62 (q, J=6.57 Hz, 2 H) 1.60 (q, J=6.91 Hz, 2 H) 0.70-0.87 (m, 1 H) 0.32-0.47 (m, 2 H) 0.10 (q, J=4.72 Hz, 2 H)

### EXAMPLE 32

### (5Z)-2-[(2,6-Dichlorophenyl)amino]-5-{[4-(1-pyrrolidinyl)-6-quinolinyl]methylidene}-1,3-thiazol-4(5H)-one

The title compound was made by following the procedure described in Example 24 as a yellow solid (72%). Pyrrolidine was used in place of piperidine. 1H NMR (400 MHz, DMSO-d₆) δ ppm 14.00 (br. s., 1 H) 13.10 (br. s, 1 H) 8.62 (s, 1 H) 8.40 (d, J=7.33 Hz, 1 H) 8.01 (s, 1 H) 7.94 (s, 2 H) 7.59 (d, J=8.08 Hz, 2 H) 7.25 (t, J=8.08 Hz, 1 H) 6.77 (d, J=7.33 Hz, 1 H) 3.91 (br. s., 4 H) 2.00 (s, 4 H)

### EXAMPLE 33

### (5Z)-2-[(2,6-Dichlorophenyl)amino]-5-{[3-(hydroxymethyl)-6-quinolinyl]methylidene}-1,3 -thiazol-4(5H)-one

LiAlH₄ (1.0 M in THF, 82 μL, 0.082 mmol) was added to the solution of the compound from Example 28b) (20 mg, 0.041 mmol) in dry THF (5 mL). The mixture was kept stirring for 2 hours, quenched with MeOH, concentrated under vacuo and purified through preparative HPLC (YMC 75×30 mm column, 0.1% TFA in water and 0.1% TFA in acetonitrile ) to afford the title compound as a yellow solid (11 mg, 65%). 1H NMR (400 MHz, DMSO-d₆) δ ppm 8.82 (s, 1 H) 8.22 (s, 1 H) 7.70-8.09 (m, 3 H) 7.26-7.47 (m, 3 H) 6.98 (d, J=14.15 Hz,1 H) 4.71 (s, 2 H)

### EXAMPLE 34

### 6-{(Z)-[2-[(2,6-Dichlorophenyl)amino]-4-oxo-1,3-thiazol-5(4H)-ylidene]methyl}-N-methyl-3-quinolinecarboxamide hydrochloride

Methylamine (2.0 M in methanol, 2 mL, 4 mmol) was added to a solution of the compound from Example 28b) (20 mg, 0.042 mmol) in water (2 mL). The mixture was kept stirring for overnight at ambient temperature, concentrated and acidified. The precipitate was collected, washed with water and dried under vacuo to afford the title compound as a yellow solid (12 mg, 63%). 1H NMR (400 MHz, DMSO-d₆) δ ppm 9.37 (d, J=1.77 Hz, 1 H) 9.05 (s,1 H) 8.98 (d, J=4.29 Hz, 1 H) 8.26 (s, 1 H) 8.20 (d, J=8.84 Hz, 1 H) 8.02 (dd, J=8.84, 1.52 Hz, 1 H) 7.94 (s, 1 H) 7.84 (br. s., 1 H) 7.59 (d, J=8.08 Hz, 2 H) 7.25 (t, J=8.21 Hz, 1 H) 2.85 (d, J=4.55 Hz, 3 H)

### EXAMPLE 35

### 6-{(Z)-[2-[(2,6-Dichlorophenyl)amino]-4-oxo-1,3-thiazol-5(4H)-ylidene]methyl}-N,N-dimethyl-3-quinolinecarboxamide

The mixture of the compound from Example 29 (34 mg, 0.076 mmol), diisopropylethylamine (26 μL, 0.15 mmol), dimethylamine hydrochloride (6.2 μL, 0.076 mmol) and 1-(3-dimethylaminopropyl)-3-ethylcarbodiimide hydrochloride (15 mg, 0.076 mmol) in dry methylene chloride (5 mL) was kept stirring overnight at ambient temperature, then heated to 40° C for 5 hours. The product was diluted with methylene chloride (30 mL), washed with 1 N HCl, saturated NaHCO₃ and brine subsequently. The organic layer was dried over MgSO₄, filtered, concentrated over vacuo and purified via flash chromatography (0-10% methanol in methylene chloride) to afford the title compound as a yellow solid (14 mg, 40%). 1H NMR (400 MHz, DMSO-d₆) δ ppm 8.94 (d, J=2.02 Hz, 1 H) 8.60 (d, J=1.77 Hz, 1 H) 8.19 (s, 1 H) 8.12 (d, J=8.59 Hz, 1 H) 7.89-7.98 (m, 2 H) 7.58 (d, J=8.34 Hz, 2 H) 7.24 (t, J=8.21 Hz, 1 H) 3.05 (s, 3 H) 2.99 (s, 3 H)

### EXAMPLE 36

### (5Z)-2-[(2,6-Dichlorophenyl)amino]-5-[(4-phenyl-6-quinolinyl)methylidene]-1,3-thiazol-4(5H)-one trifluoroacetate

The mixture of the compound from Example 22d) (20 mg, 0.046 mmol), tetrakis(triphenylphosphine)palladium (2.2 mg, 0.0018 mmol), potassium carbonate (38 mg, 0.28 mmol) and phenylboronic acid (5.6 mg, 0.046 mmol) in DME/H₂O=3/1(2 mL) was heated to 120° C. in a Biotage Initiator microwave synthesizer for one hour. The product was concentrated and purified via preparative HPLC (YMC 75×30 mm column, 0.1% TFA in water and 0.1% TFA in acetonitrile ) to afford the title compound as yellow solid (10 mg, 45%). 1H NMR (400 MHz, DMSO-d₆) δ ppm 12.99 (br. s., 1 H) 9.01 (d, J=4.29 Hz, 1 H) 8.17 (d, J=8.84 Hz, 1 H) 8.01 (dd, J=8.84, 2.02 Hz, 1 H) 7.85-7.91 (m, 2 H) 7.68 (d, J=8.08 Hz, 2 H) 7.59-7.66 (m, 2 H) 7.55-7.58 (m, 2 H) 7.48 (d, J=7.07 Hz, 2 H) 7.38-7.43 (m, 1 H) 7.23 (t, J=7.71 Hz, 2 H)

### EXAMPLE 37

### (5Z)-2-[(3-Chloro-2-biphenylyl)amino]-5-[(4-phenyl-6-quinolinyl)methylidene]-1,3-thiazol -4(5H)-one trifluoroacetate

The title compound was made by following the procedure described in Example 36 as a yellow solid (50%). 2 equivalent of phenylboronic acid was used. 1H NMR (400 MHz, DMSO-d₆) δ ppm 12.67 (br. s., 1 H) 9.02 (d, J=4.55 Hz, 1 H) 8.16 (d, J=8.84 Hz, 1 H) 7.99 (dd, J=8.84,2.02 Hz, 1 H) 7.86 (d, J=1.77 Hz, 1 H) 7.76 (s,1 H) 7.69 (dd, J=5.94, 3.66 Hz, 1 H) 7.58 (d, J=4.55 Hz, 1 H) 7.42-7.53 (m, 5 H) 7.22-7.40 (m, 7 H)

### EXAMPLE 38

### (5Z)-2-[(2,6-Dichlorophenyl)amino]-5-[(4-methyl-6-quinolinyl)methylidene]-1,3-thiazol-4(5H)-one

a) 6-Ethenyl-4-methylquinoline. Methylmagnesium bromide (3.0 M in diethyl ether, 0.42 mL, 1.26 mmol) was added dropwise to the mixture of the compound from Example 22b) (100 mg, 0.53 mmol), Fe2(acac)3 (9.3 mg, 0.026 mmol) in THF (2 mL) and NMP (0.2 mL). The mixture was kept stirring for an hour, quenched by 1N HCl and extracted with ethyl acetate. The extract was washed with saturated NaHCO₃ and brine, dried over MgSO₄, filtered, concentrated under vacuo and purified via flash chromatography (0-100% ethyl acetate in hexane) to afford the title compound as a yellow solid (50 mg, 56%). MS(ES+) m/e 170 [M+H]⁺.
b) 4-Methyl-6-quinolinecarbaldehyde. The title compound was made by following the procedure described in Example 22c) as a white solid (79%). The compound from Example 38a) was used in place of the compound from Example 22b). 1H NMR (400 MHz, CHLOROFORM-d) δ ppm 10.22 (s, 1 H) 8.89 (d, J=4.55 Hz, 1 H) 8.53 (d, J=1.26 Hz, 1 H) 8.21 (d, J=8.59 Hz, 1 H) 8.18 (dd, J=8.59, 1.77 Hz, 1 H) 7.35 (d, J=3.54 Hz, 1 H) 2.81 (s, 3 H).
c) (5Z)-2-[(2,6-Dichlorophenyl)amino]-5-[(4-methyl-6-quinolinyl)methylidene]-1,3-thiazol-4(5H)-one. The title compound was made by following the procedure described in Example 24 as a yellow solid (41%). The compound from Example 38b) was used in place of the compound from 22 d. 1H NMR (400 MHz, DMSO-d₆) δ ppm 13.02 (br. s., 1 H) 8.79 (d, J=4.29 Hz, 1 H) 8.38 (s, 1 H) 8.06 (d, J=8.84 Hz, 1 H) 7.99 (s, 1 H) 7.75 (d, J=8.84 Hz, 1 H) 7.58 (d, J=8.08 Hz, 2 H) 7.42 (d, J=4.04 Hz, 1 H) 7.25 (t, J=8.21 Hz, 1 H) 2.66 (s, 3 H)

### EXAMPLE 39

### (5Z)-2-[(2,6-Dichlorophenyl)amino]-5-{[4-(1-methylethyl)-6-quinolinyl]methylidene}-1,3-thiazol-4(5H)-one

The title compound was made by following the method described in Example 38 as a yellow solid (45%). Isopropylmagnesium bromide was used in place of methylmagnesium bromide. 1H NMR (400 MHz, DMSO-d₆) δ ppm 13.02 (br. s., 1 H) 8.87 (d, J=4.55 Hz, 1 H) 8.51 (s, 1 H) 8.09 (d, J=8.84 Hz, 1 H) 8.04 (s, 1 H) 7.77 (dd, J=8.72, 1.14 Hz, 1 H) 7.59 (d, J=8.34 Hz, 2 H) 7.48 (d, J=4.55 Hz, 1 H) 7.25 (t, J=8.08 Hz, 1 H) 3.74 (qq, J=6.82 Hz, 1 H) 1.31 (d, J=6.82 Hz, 6 H)

### EXAMPLE 40

### (5Z)-2-[(2,6-Dichlorophenyl)amino]-5-[(4-ethyl-6-quinolinyl)methylidene]-1,3-thiazol-4(5H)-one

The title compound was made by following the method described in Example 38 as a yellow solid (45%). Ethylmagnesium bromide was used in place of methylmagnesium bromide. 1H NMR (400 MHz, DMSO-d₆) δ ppm 13.02 (br. s., 1 H) 8.83 (d, J=4.04 Hz, 1 H) 8.43 (s, 1 H) 7.95-8.18 (m, 2 H) 7.77 (d, J=8.59 Hz, 1 H) 7.59 (d, J=8.08 Hz, 2 H) 7.43 (d, J=4.04 Hz, 1 H) 7.25 (t, J=7.96 Hz, 1 H) 3.07 (q, J=7.07 Hz, 2 H) 1.26 (t, J=7.07 Hz, 3 H)

### EXAMPLE 41

### 6-{(Z)-[2-[(2,6-Dichlorophenyl)amino]-4-oxo-1,3-thiazol-5(4H)-ylidene]methyl}-4-quinolinecarbonitrile

a) 6-Ethenyl-4-quinolinecarbonitrile. The mixture of the compound from Example 22b) (100 mg, 0.53 mmol), Zn(CN)₂ (37 mg, 0.32 mmol), Pd₂(dba)₃ (19.4 mg, 0.02 mmol), dppf (23.5 mg, 0.04 mmol) and Zn (4 mg, 0.064 mmol) in N,N-dimethylacetamide (2 mL) was heated to 180° C. for 5 minutes in a Biotage Initiator microwave synthesizer. The product was cooled, diluted with ethyl acetate, washed with NH40H and brine. The organic layer was dried over MgSO₄, filtered, concentrated under vacuo and purified via flash chromatography (0-10% methanol in methylene chloride) to afford a white solid (70 mg, 73%). 1H NMR (400 MHz, CHLOROFORM-d) δ ppm 8.98 (d, J=4.29 Hz, 1 H) 8.16 (d, J=8.59 Hz, 1 H) 8.06 (d, J=1.77 Hz, 1 H) 8.01 (dd, J=8.84, 1.77 Hz, 1 H) 7.73 (d, J=4.29 Hz, 1 H) 6.96 (dd, J=17.56, 10.99 Hz, 1 H) 6.04 (d, J=17.43 Hz, 1 H) 5.55 (d, J=10.86 Hz, 1 H).
b) 6-Formyl-4-quinolinecarbonitrile. The title compound was made by following the method described in Example 22c) as a white solid (59%). The compound from Example 41a) was used in place of Example 22b). MS(ES+) m/e 183 [M+H]⁺.
c) 6-{(Z)-[2-[(2,6-Dichlorophenyl)amino]-4-oxo-1,3-thiazol-5(4H)-ylidene]methyl}-4-quinolinecarbonitrile. The title compound was made by following the method described in Example 6f) as a yellow solid (14%). The compound from Example 41b) was used in place of the compound from Example 6d). 1H NMR (400 MHz, DMSO-d₆) δ ppm 13.13 (s, 1 H) 9.13 (d, J=4.55 Hz, 1 H) 8.22-8.31 (m, 2 H) 8.17 (d, J=4.29 Hz, 1 H) 8.05 (s, 1 H) 8.00 (d, J=8.84 Hz, 1 H) 7.58 (d, J=8.34 Hz, 2 H) 7.24 (t, J=8.08 Hz, 1 H)

### EXAMPLE 42

### (5Z)-2-[(2,6-Dichlorophenyl)amino]-5-{[4-(4-pyridinyl)-6-quinolinyl]methylidene}-1,3-thiazol-4(5H)-one

The title compound was made by following the method described in Example 37 as a yellow solid (24%). 4-pyridinylboronic acid was used in place of phenylboronic acid. 1H NMR (400 MHz, DMSO-d₆) δ ppm 13.02 (br. s., 1 H) 9.06 (d, J=4.29 Hz, 1 H) 8.63 (d, J=4.80 Hz, 2 H) 8.21 (d, J=8.84 Hz, 1 H) 7.94-8.00 (m, 1 H) 7.87-7.94 (m, 2 H) 7.68 (d, J=5.31 Hz, 2 H) 7.59-7.65 (m, 3 H) 7.33 (t, J=8.21 Hz, 1 H)

### EXAMPLE 43

### (5Z)-2-[(2,6-Dichlorophenyl)amino]-5-{[4-(3-pyridinyl)-6-quinolinyl]methylidene}-1,3-thiazol-4(5H)-one

The title compound was made by following the method described in Example 37 as a yellow solid (24%). 3-pyridinylboronic acid was used in place of phenylboronic acid. 1H NMR (400 MHz, DMSO-d₆) δ ppm 12.99 (br. s., 1 H) 9.02 (d, J=4.55 Hz, 1 H) 8.70 (d, J=1.52 Hz, 1 H) 8.65 (dd, J=4.80, 1.52 Hz, 1 H) 8.18 (d, J=8.84 Hz, 1 H) 7.93-8.03 (m, 2 H) 7.89 (s, 1 H) 7.85 (d, J=1.52 Hz, 1 H) 7.66 (d, J=8.08 Hz, 2 H) 7.61 (d, J=4.29 Hz, 1 H) 7.38 (t, J=8.21 Hz, 1 H) 7.17 (dd, J=7.71, 4.93 Hz, 1 H)

### EXAMPLE 44

### Ethyl 6-{(Z)-[2-[(2,6-dichlorophenyl)amino]-4-oxo-1,3-thiazol-5(4H)-ylidene]methyl}-4-(4-morpholinyl)-3-quinolinecarboxylate

a) Ethyl 6-bromo-4-(4-morpholinyl)-3-quinolinecarboxylate. The mixture of the compound from Example 21a) (200 mg, 0.64 mmol) and morpholine (111 mg, 1.28 mmol) in methanol (2 mL) was heated to 120° C. for 5 minutes in a Biotage Initiator microwave synthesizer. The product was diluted with ethyl acetate and washed with 1N HCl, saturated NaHCO₃ and brine subsequently. The organic layer was dried over MgSO₄, filtered, concentrated under vacuo and purified via flash chromatography (0-10% methanol in methylene chloride) to afford a white solid (150 mg, 64%). MS(ES+) m/e 365 [M+H]⁺.
b) Ethyl 6-ethenyl-4-(4-morpholinyl)-3-quinolinecarboxylate. The title compound was made by following the method described in Example 22b) as a yellow solid (70%). The compound from Example 44a) was used in place of the compound from Example 22a). 1H NMR (400 MHz, CHLOROFORM-d) δ ppm 8.86 (s, 1 H) 8.09 (d, J=2.02 Hz, 1 H) 8.04 (d, J=8.84 Hz, 1 H) 7.89 (dd, J=8.84, 2.02 Hz, 1 H) 6.92 (dd, J=1 7.56, 10.99 Hz, 1 H) 5.92 (d, J=1 7.68 Hz, 1 H) 5.44 (d, J=11.12 Hz, 1 H) 4.48 (d, J=7.07 Hz, 2 H) 3.99 (t, J=4.29 Hz, 4 H) 3.36 (t, J=4.80 Hz, 4 H) 1.47 (t, J=7.07 Hz, 3 H).
c) Ethyl 6-formyl-4-(4-morpholinyl)-3-quinolinecarboxylate. The title compound was made by following the method described in Example 22c) as a white solid (71%). The compound from Example 44b) was used in place of the compound from Example 22b). 1H NMR (400 MHz, CHLOROFORM-d) δ ppm 10.10 (s, 1 H) 8.89 (s, 1 H) 8.59 (d, J=1.01 Hz, 1 H) 8.08 (dd, J=8.59, 1.52 Hz, 1 H) 8.05 (d, J=8.59 Hz, 1 H) 4.43 (q, J=7.16 Hz, 2 H) 3.95 (t, J=4.80 Hz, 4 H) 3.34 (t, J=4.55 Hz, 3 H) 1.41 (t, J=7.20 Hz, 3 H).
d) Ethyl 6-{(Z)-[2-[(2,6-dichlorophenyl)amino]-4-oxo-1,3-thiazol-5(4H)-ylidene]methyl}-4-(4-morpholinyl)-3-quinolinecarboxylate. The title compound was made by following the method described in Example 22d) as a white solid (24%). The compound from Example 44c) was used in place of the compound from Example 22c). 1H NMR (400 MHz, DMSO-d₆) δ ppm 13.09 (s, 1 H) 8.83 (s, 1 H) 8.25 (d, J=1.52 Hz, 1 H) 8.04-8.10 (m, 1 H) 7.97-8.03 (m, 1 H) 7.96 (s, 1 H) 7.52-7.62 (m, 2 H) 7.24 (t, J=8.08 Hz, 1 H) 4.41 (q, J=7.07 Hz, 2 H) 3.49-3.61 (m, 4 H) 3.07-3.18 (m, 4 H) 1.37 (t, J=7.20 Hz, 3 H)

### EXAMPLE 45

### Ethyl 6-{(Z)-[2-[(2,6-dichlorophenyl)amino]-4-oxo-1,3-thiazol-5(4H)-ylidene]methyl}-4-(4-methyl-1-piperazinyl)-3-quinolinecarboxylate

The title compound was made by following the method described in Example 44 as a yellow solid (70%). 1-methylpiperazine was used in place of morpholine. 1H NMR (400 MHz, DMSO-d₆) δ ppm 12.59 (br. s., 1 H) 8.79 (s, 1 H) 8.12 (s, 1 H) 8.05 (d, J=8.59 Hz, 1 H) 7.88-7.96 (m, 2 H) 7.55 (d, J=8.08 Hz, 2 H) 7.22 (t, J=7.96 Hz, 1 H) 4.38 (q, J=7.07 Hz, 2 H) 3.19 (s, 4 H) 2.31 (s, 4 H) 1.36 (t, J=7.07 Hz,3 H)

### EXAMPLE 46

### 6-{(Z)-[2-[(2,6-Dichlorophenyl)amino]-4-oxo-1,3-thiazol-5(4H)-ylidene]methyl}-4-(4-morpholinyl)-3-quinolinecarboxamide trifluoroacetate

a) 6-{(Z)-[2-[(2,6-Dichlorophenyl)amino]-4-oxo-1,3-thiazol-5(4H)-ylidene]methyl}-4-(4-morpholinyl)-3-quinolinecarboxylic acid. NaOH (6N in water, 2 mL, 12 mmol) was added to the solution of the compound from Example 44d) (50 mg, 0.09 mmol) in methanol (2 mL). The mixture was kept stirring at ambient temperature for 4 hours and acidified with 1 N HCl. The precipitate was collected, washed with water and dried under vaccuo to afford the title compound as a yellow solid (40 mg, 85%). MS(ES+) m/e 429 [M+H]⁺.
b) 6-{(Z)-[2-[(2,6-Dichlorophenyl)amino]-4-oxo-1,3-thiazol-5(4H)-ylidene]methyl}-4-(4-morpholinyl)-3-quinolinecarboxamide trifluoroacetate. 1,1′-Carbonyidiimidazole (30.7 mg, 0.19 mmol) was added to the solution of the compound from Example 46a) (50 mg, 0.095 mmol) in DMF (2 mL). The mixture was heated to 50° C. for half hour, then ammonium hydroxide (2 mL) was added. The product was kept at 50° C. for 2 hours, concentrated under vacuo and purified via preparative HPLC (YMC 75×30 mm column, 0.1% TFA in water and 0.1% TFA in acetonitrile ) to afford the title compound as yellow solid (15 mg, 29%). 1H NMR (400 MHz, DMSO-d₆) δ ppm 13.08 (br. s., 1 H) 8.68 (s, 1 H) 8.23 (s, 1 H) 8.16 (s, 1 H) 8.01-8.06 (m, 1 H) 7.92-8.00 (m,2 H) 7.84 (s, 1 H) 7.57 (d, J=8.08 Hz, 2 H) 7.25 (t, J=8.08 Hz, 1 H) 3.56 (s, 4 H) 3.21 (s, 4 H)

### EXAMPLE 47

### N-{4-Chloro-3-[((5Z)-5-{[4-(4-morpholinyl)-6-quinolinyl]methylidene}-4-oxo-4,5-dihydro-1,3-thiazol-2-yl)amino]phenyl}cyclobutanecarboxamide

a) N-(3-Amino-4-chlorophenyl)cyclobutanecarboxamide. Cyclobutylcarbonyl chloride (3.64 mL, 31.9 mmol) was added dropwise to an ice-cooled, stirred solution of 4-chloro-3-nitroaniline (5.00 g, 29.0 mmol) and pyridine (3.5 mL, 43.2 mmol) in dichloromethane (30 mL) under argon. The mixture was allowed to warm to room temperature and stirred 1 h, then the solevent removed under reduced pressure. 0.5 M aqueous potassium carbonate (10 mL) and methanol (30 mL) was added and the mixture stirred 0.5 h, then diluted with water (200 mL) and extracted with ethyl acetate. The extracts were washed (1M aq HCl, water, brine), dried (MgSO₄) and evaporated to dryness under reduced pressure. A solution of the crude amide in methanol (150 mL) was stirred with Raney® nickel (˜0.5 g) under 1 atm of hydrogen for 18 h. After removal of the hydrogen, the mixture was filtered through a PTFE micropore filter, then evaporated under reduced pressure to give the title compound (6.52 g, 100%) as an oil. 1H NMR (400 MHz, DMSO-d₆) δ 1.80 (m,1H), 1.92 (m,1H), 2.03-2.11 (m, 2H), 2.14-2.24 (m, 2H), 3.18 (m, 1H), 5.32 (s, 2H), 6.73 (dd, J=8.6,2.5 Hz, 1H), 7.05 (d, J=8.6 Hz, 1H), 7.20 (d, J=2.5 Hz, 1H), 9.56 (s, 1H).
b) N-{4-Chloro-3-[(4-oxo-4,5-dihydro-1,3-thiazol-2-yl)amino]phenyl}cyclobutane-carboxamide. A mixture of the compound from example 47(a) (6.55 g, 29.0 mmol), 2-(methylthio)-1,3-thiazol-4(5H)-one (A. I. Khodair, J. Heterocyclic Chem., 2002, 39, 1153; 5.30 g, 36.0 mmol) and ethanol (36 mL) was heated under reflux for 18 h, then cooled. The solid was filtered, washed with ethanol and dried to give the title compound (8.33 g, 89%) as a brown solid. 1H NMR (400MHz, DMSO-d₆) δ 1.82 (m, 1H), 1.95 (m, 1H), 2.10 (m, 2H), 2.21 (m, 2H), 3.21 (m, 1H), 4.03 (s, 2H), 7.30 (d, J=8.8 Hz, 1H), 7.38 (d, J=8.8 Hz, 1H), 7.45 (s, 1H), 9.85 (s, 1H), 11.99 (br s,1 H).
c) N-{4-Chloro-3-[((5Z)-5-{[4-(4-morpholinyl)-6-quinolinyl]methylidene}-4-oxo-4,5-dihydro-1,3-thiazol-2-yl)amino]phenyl}cyclobutanecarboxamide. The title compound was made by following the method described in Example 22d) as a yellow solid (11%). The compound from example 47(b) was used in place of 2-[(2,6-dichlorophenyl)amino]-1,3-thiazol-4(5H)-one and morpholine was used in place of sodium acetate. 1H NMR (400 MHz, DMSO-d₆) δ ppm 12.79 (br. s., 1 H) 9.95 (s, 1 H) 8.74 (d, J=5.05 Hz, 1 H) 8.06 (d, J=1.52 Hz, 1 H) 7.98-8.03 (m, 1 H) 7.86-7.94 (m, 2 H) 7.62 (d, J=2.27 Hz, 1 H) 7.43-7.50 (m, 1 H) 7.39 (dd, J=8.00, 4.00 Hz, 1 H) 7.03 (d, J=5.05 Hz, 1 H) 3.52-3.60 (m, 4 H) 3.16-3.27 (m, 2 H) 3.02-3.13 (m, J=3.79 Hz, 4 H) 2.15-2.27 (m, 2 H) 2.04-2.13 (m, 2 H) 1.87 -1.98 (m, 1 H)

### EXAMPLE 48

### N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}cyclobutanecarboxamide

The title compound was made by following the method described in Example 22d) as a yellow solid (12%). The compound from example 47(b) was used in place of 2-[(2,6-dichlorophenyl)amino]-1,3-thiazol-4(5H)-one and 1-methylpiperazine was used in place of sodium acetate. 1H NMR (400 MHz, DMSO-d₆) δ ppm 9.93 (s, 1 H) 8.71 (d, J=5.05 Hz, 1 H) 7.95-8.03 (m, 2 H) 7.84-7.92 (m, 2 H) 7.50 (d, J=2.02 Hz, 1 H) 7.42-7.46 (m, 2 H) 7.01 (d, J=5.05 Hz, 1 H) 3.16-3.24 (m, 1 H) 3.11 (br. s.,4 H) 2.36 (br. s.,4 H) 2.17-2.22 (m, 1 H) 2.15 (s, 3 H) 2.01-2.10 (m, 2 H) 1.86-1.96 (m, 1 H) 1.72-1.83 (m, 1 H)

### EXAMPLE 49

### 6-{(Z)-[2-[(2,6-Dichlorophenyl)amino]-4-oxo-1,3-thiazol-5(4H)-ylidene]methyl}-4-(4-methyl-1-piperazinyl)-3-quinolinecarboxamide trifluoroacetate

The title compound was made by following the method described in Example 46 as a white solid (50%). The compound from Example 45 was used in place of the compound from Example 44d). 1H NMR (400 MHz, DMSO-d₆) δ ppm 13.10 (br. s., 2 H) 10.06 (br. s., 3 H) 8.79 (s, 3 H) 8.30 (d, J=14.15 Hz, 2 H) 8.04-8.13 (m, 2 H) 7.98 (s, 1 H) 7.84 (dd, J=8.84, 1.52 Hz, 1 H) 7.71 (d, J=1.26 Hz, 1 H) 7.60 (d, J=8.34 Hz, 2 H) 7.25 (t, J=8.21 Hz, 1 H) 3.52 (s, 4 H) 3.48 (s, 4 H) 2.92 (s, 3 H)

### EXAMPLE 50

### (5Z)-2-[(2,6-Dichlorophenyl)amino]-5-{[4-(1-piperazinyl)-6-quinolinyl]methylidene}-1,3-thiazol-4(5H)-one

The title compound was made by following the method described in Example 22d) as a yellow solid (30%). Piperazine was used in place of sodium acetate. 1H NMR (400 MHz, DMSO-d₆) δ ppm 8.71 (d, J=4.55 Hz, 1 H) 8.09 (s, 1 H) 7.98 (d, J=8.34 Hz, 1 H) 7.81 (br. s., 1 H) 7.70 (br. s., 1 H) 7.29-7.59 (m, 2 H) 6.86-7.22 (m, 2 H) 3.21 (br. s., 4 H) 3.05 (br. s., 4 H)

### EXAMPLE 51

### N-{4-Chloro-3-[((5Z)-4-oxo-5-{[4-(1-piperazinyl)-6-quinolinyl]methylidene}-4,5-dihydro-1,3-thiazol-2-yl)amino]phenyl}cyclobutanecarboxamide

The title compound was made by following the method described in Example 22d) as a yellow solid (19%). The compound from example 47(b) was used in place of 2-[(2,6-dichlorophenyl)amino]-1,3-thiazol-4(5H)-one and piperazine was used in place of sodium acetate. 1H NMR (400 MHz, DMSO-d₆) δ ppm 9.92 (s, 1 H) 8.70 (d, J=5.05 Hz, 1 H) 8.08 (s, 1 H) 7.97 (d, J=8.59 Hz, 1 H) 7.84 (d, J=8.59 Hz, 1 H) 7.74 (s, 1 H) 7.34-7.47 (m, 3 H) 7.01 (d, J=4.80 Hz, 1 H) 3.20-3.29 (m, 1 H) 3.17 (s, 3 H) 3.13 (br. s.,4 H) 2.89 (br. s., 4 H) 2.16-2.28 (m, 2 H) 2.03-2.14 (m, 2 H) 1.87-1.98 (m, 1 H) 1.78 (q, J=9.60 Hz, 1 H)

### EXAMPLE 52

### Ethyl 6-{(Z)-[2-[(2,6-dichlorophenyl)amino]-4-oxo-1,3-thiazol-5(4H)-ylidene]methyl}-4-(dimethylamino)-3-quinolinecarboxylate

The title compound was made by following the method described in Example 21d) as a yellow solid (17%). Dimethylamine was used in place of sodium acetate. 1H NMR (400 MHz, DMSO-d₆) δ ppm 13.07 (br. s., 1 H) 8.84 (s, 1 H) 8.63 (s, 1 H) 7.96-8.05 (m, 2 H) 7.83-7.96 (m, 1 H) 7.58 (d, J=8.08 Hz, 2 H) 7.25 (t, J=8.21 Hz, 1 H) 4.36 (q, J=7.07 Hz, 2 H) 3.44 (s, 6 H) 1.35 (t, J=7.07 Hz, 3 H)

### EXAMPLE 53

### Ethyl 6-{(Z)-[2-[(2,6-dichlorophenyl)amino]-4-oxo-1,3-thiazol-5(4H)-ylidene]methyl}-4-(methylamino)-3-quinolinecarboxylate

The title compound was made by following the method described in Example 21d) as a yellow solid (52%). Methylamine was used in place of sodium acetate. 1H NMR (400 MHz, DMSO-d₆) δ ppm 12.99 (br. s., 1 H) 9.06 (br. s., 1 H) 8.82 (s, 1 H) 8.63 (d, J=1.26 Hz, 1 H) 7.92 (s, 1 H) 7.86 (d, J=8.84 Hz, 1 H) 7.72 (d, J=8.84 Hz, 1 H) 7.58 (d, J=8.08 Hz, 2 H) 7.24 (t, J=8.08 Hz, 1 H) 4.33 (q, J=7.07 Hz, 2 H) 3.28 (d, J=5.05 Hz, 3 H) 1.34 (t, J=7.07 Hz, 3 H)

### EXAMPLE 54 (GSK918678A, DC205761-110A1)

### EXAMPLE 54

### Ethyl 6-{(Z)-[2-({2-chloro-5-[(cyclobutylcarbonyl)amino]phenyl}amino)-4-oxo-1,3-thiazol-5(4H)-ylidene]methyl}-4-(dimethylamino)-3-quinolinecarboxylate trifluoroacetate

The title compound was made by following the method described in Example 53 as a yellow solid (27%). Dimethylamine was used in place of sodium acetate and the compound from example 47(b) was used in place of 2-[(2,6-dichlorophenyl)amino]-1,3-thiazol-4(5H)-one. 1H NMR (400 MHz, DMSO-d₆) δ ppm 12.82 (br. s., 1 H) 9.98 (s, 1 H) 8.83 (s, 1 H) 8.56 (s, 1 H) 7.99-8.05 (m, 1 H) 7.91-7.97 (m, 2 H) 7.55 (d, J=2.27 Hz, 1 H) 7.44-7.49 (m, 1 H) 7.35-7.43 (m, 1 H) 4.36 (q, J=7.07 Hz, 2 H) 3.35 (s, 6 H) 3.19-3.27 (m, 1 H) 2.16-2.28 (m,2 H) 2.05-2.15 (m,2 H) 1.88-2.01 (m, 1 H) 1.72-1.84 (m, 1 H)

### EXAMPLE 55

### Ethyl 6-{(Z)-[2-({2-chloro-5-[(cyclobutylcarbonyl)amino]phenyl}amino)-4-oxo-1,3-thiazol-5(4H)-ylidene]methyl}-4-(methylamino)-3-quinolinecarboxylate

The title compound was made by following the method described in Example 52 as a yellow solid (41%). Methylamine was used in place of sodium acetate and the compound from example 47(b) was used in place of 2-[(2,6-dichlorophenyl)amino]-1,3-thiazol-4(5H)-one. 1H NMR (400 MHz, DMSO-d₆) δ ppm 12.74 (br. s., 1 H) 9.94 (s, 1 H) 9.04 (br. s., 1 H) 8.82 (s, 1 H) 8.62 (d, J=1.26 Hz, 1 H) 7.83-7.90 (m, 2 H) 7.76 (dd, J=8.84, 1.52 Hz, 1 H) 7.52 (d, J=2.02 Hz, 1 H) 7.37-7.48 (m, 2 H) 4.32 (q, J=7.07 Hz, 2 H) 3.28 (d, J=5.31 Hz, 3 H) 3.17-3.25 (m, 1 H) 2.16-2.29 (m, 2 H) 2.04-2.15 (m,2 H) 1.86-1.99 (m,1 H) 1.68-1.85 (m,1 H)

### EXAMPLE 56

### Ethyl 6-{(Z)-[2-({2-chloro-5-[(cyclobutylcarbonyl)amino]phenyl}amino)-4-oxo-1,3-thiazol-5(4H)-ylidene]methyl}-4-(4-morpholinyl)-3-quinolinecarboxylate trifluoroacetate

The title compound was made by following the method described in Example 55 as a yellow solid (32%). Morpholine was used in place of methylamine. 1H NMR (400 MHz, DMSO-d₆) δ ppm 12.85 (br. s., 1 H) 9.92 (s, 1 H) 8.84 (s, 1 H) 8.23 (d, J=1.77 Hz, 1 H) 7.99-8.11 (m, 2 H) 7.92 (s, 1 H) 7.62 (d, J=2.27 Hz, 1 H) 7.40-7.47 (m, 1 H) 7.35 (dd, J=8.84, 2.53 Hz, 1 H) 4.40 (q, J=7.24 Hz, 2 H) 3.50-3.62 (m,4 H) 3.08-3.23 (m, 4 H) 2.13-2.26 (m, 2 H) 2.01-2.12 (m, 2 H) 1.84-1.97 (m,1 H) 1.69-1.82 (m, 1 H) 1.36 (t, J=7.07 Hz, 3 H)

### EXAMPLE 57

### Ethyl 6-{(Z)-[2-[(2,6-dichlorophenyl)amino]-4-oxo-1,3-thiazol-5(4H)-ylidene]methyl}-4-(4-pyridinyl)-3-quinolinecarboxylate trifluoroacetate

a) Ethyl 6-ethenyl-4-(4-pyridinyl)-3-quinolinecarboxylate. The mixture of the compound from Example 21b) (180 mg, 0.69 mmol), tetrakis(triphenylphosphine)palladium (32 mg, 0.0028 mmol), sodium carbonate (219 mg, 2.07 mmol) and 4-pyridinylboronic acid (340 mg, 2.77 mmol) in DME/H₂O=3/1(2 mL) was heated to 150° C. in a Biotage Initiator microwave synthesizer for half hour. The product was concentrated and purified via flash chromatography (0-100% ethyl acetate in hexane) to afford the title compound as yellow solid (80 mg, 38%). 1H NMR (400 MHz, CHLOROFORM-d) δ ppm 9.40 (s, 1 H) 8.63-8.91 (m, 2 H) 8.19 (d, J=8.84 Hz, 1 H) 8.01 (dd, J=8.84, 2.02 Hz, 1 H) 6.74 (dd, J=17.68, 10.86 Hz, 1 H) 5.84 (d, J=17.43 Hz, 1 H) 5.40 (d, J=11.12 Hz, 1 H) 4.18 (q, J=7.24 Hz, 2 H) 1.09 (t, J=7.20 Hz, 3 H).
b) Ethyl 6-formyl-4-(4-pyridinyl)-3-quinolinecarboxylate. The title compound was made by following the method described in Example 22c) as a white solid (85%).

The compound from Example 57a) was used in place of 4-chloro-6-ethenylquinoline (Example 22b). MS(ES+) m/e 307 [M+H]⁺.
c) Ethyl 6-{(Z)-[2-[(2,6-dichlorophenyl)amino]-4-oxo-1,3-thiazol-5(4H)-ylidene]methyl}-4-(4-pyridinyl)-3-quinolinecarboxylate trifluoroacetate. The title compound was made by following the method described in Example 22(d) as a yellow solid (17%). The compound from Example 57b) was used in place of the compound from Example 22c). 1H NMR (400 MHz, DMSO-d₆) δ ppm 13.01 (br. s., 1 H) 9.36 (s, 1 H) 8.60 (d, J=4.80 Hz, 2 H) 8.25 (d, J=8.84 Hz, 1 H) 8.05 (dd, J=8.84, 1.77 Hz, 1 H) 7.85 (s, 1 H) 7.64 (d, J=8.08 Hz, 2 H) 7.45-7.55 (m, 3 H) 7.35 (t, J=8.08 Hz, 1 H) 4.09 (q, J=6.99 Hz, 2 H) 0.96 (t, J=7.07 Hz, 3 H)

### EXAMPLE 58

### Ethyl 6-{(Z)-[2-({2-chloro-5-[(cyclobutylcarbonyl)amino]phenyl}amino)-4-oxo-1,3-thiazol-5(4H)-ylidene]methyl}-4-(4-pyridinyl)-3-quinolinecarboxylate trifluoroacetate

The title compound was made by following the method described in Example 57c) as a yellow solid (41%). Dimethylamine was used in place of sodium acetate and the compound from example 47(b) was used in place of 2-[(2,6-dichlorophenyl)amino]-1,3-thiazol-4(5H)-one. 1H NMR (400 MHz, DMSO-d₆) δ ppm 12.75 (br. s., 1 H) 10.00 (s, 1 H) 9.35 (s,1 H) 8.61 (d, J=5.56 Hz, 1 H) 8.25 (d, J=8.84 Hz, 1 H) 8.05 (dd, J=8.84, 1.77 Hz, 1 H) 7.81 (s, 1 H) 7.44-7.62 (m, 6 H) 4.09 (q, J=7.24 Hz, 2 H) 3.13-3.33 (m, 1 H) 2.17-2.29 (m, 2 H) 2.04-2.14 (m,2 H) 1.87-1.99 (m, 1 H) 1.73-1.83 (m, 1 H) 0.96 (t, J=7.07 Hz, 3 H)

### EXAMPLE 59

### 6-{(Z)-[2-[(2,6-Dichlorophenyl)amino]-4-oxo-1,3-thiazol-5(4H)-ylidene]methyl}-4-(4-pyridinyl)-3-quinolinecarboxamide trifluoroacetate

The title compound was made by following the method described in Example 46 as a yellow solid (70%). The compound from Example 57 was used in place of the compound from Example 44d). 1H NMR (400 MHz, DMSO-d₆) δ ppm 13.00 (br. s., 1 H) 9.04 (s, 1 H) 8.52 (d, J=5.30 Hz, 2 H) 8.21 (d, J=8.84 Hz, 1 H) 7.92-8.03 (m, 2 H) 7.86 (s, 1 H) 7.64 (d, J=8.08 Hz, 3 H) 7.56 (d, J=1.77 Hz, 1 H) 7.32-7.41 (m, 3 H)

### EXAMPLE 60

### 6-{(Z)-[2-[(2,6-Dichlorophenyl)amino]-4-oxo-1,3-thiazol-5(4H)-ylidene]methyl}-4-quinolinecarboxylic acid trifluoroacetate.

a) 6-Ethenyl-4-quinolinecarboxylic acid. NaOH (6N, 0.5 mL, 3 mmol) was added to the solution of the compound from Example 41a) (200 mg, 1.1 mmol) in methanol (0.5 mL). The mixture was heated to 150° C. for 5 minutes in a Biotage Initiator microwave synthesizer. The product was neutralized. The precipitate was collected and dried under vacuo to afford the title compound as a grey solid (140 mg, 64%). 1H NMR (400 MHz, DMSO-d₆) δ ppm 13.94 (br. s., 1 H) 9.00 (d, J=4.29 Hz, 1 H) 8.64 (s, 1 H) 8.09 (s, 2 H) 7.93 (d, J=4.29 Hz, 1 H) 6.99 (dd, J=17.68, 10.86 Hz, 1 H) 6.05 (d, J=17.43 Hz, 1 H) 5.47 (d, J=11.12 Hz, 1 H).
b) 6-Formyl-4-quinolinecarboxylic acid. The title compound was made by following the method described in Example 22c) as a white solid (64%). The compound from Example 60a) was used in place of the compound from Example 22b). MS(ES+) m/e 203 [M+H].
c) 6-{(Z)-[2-[(2,6-Dichlorophenyl)amino]-4-oxo-1,3-thiazol-5(4H)-ylidene]methyl}-4-quinolinecarboxylic acid trifluoroacetate. The title compound was made by following the method described in Example 24 as a white solid (9%). The compound from Example 60b) was used in place of the compound from Example 22d). 1H NMR (400 MHz, DMSO-d₆) d ppm 14.01 (br. s., 1 H) 13.05 (br. s., 1 H) 9.08 (d, J=4.29 Hz, 1 H) 8.96 (d, J=1.01 Hz, 1 H) 8.18 (d, J=8.59 Hz, 1 H) 8.00 (d, J=4.55 Hz, 1 H) 7.89-7.97 (m, 2 H) 7.58 (d, J=8.08 Hz, 2 H) 7.25 (t, J=8.08 Hz, 1 H)

### EXAMPLE 61

### 6-{(Z)-[2-[(2,6-Dichlorophenyl)amino]-4-oxo-1,3-thiazol-5(4H)-ylidene]methyl}-4-quinolinecarboxamide trifluoroacetate

The title compound was made by following the method described in Example 46b) as a yellow solid (75%). The compound from Example 60c) was used in place of the compound from Example 46a). 1H NMR (400 MHz, DMSO-d₆) δ ppm 13.03 (br. s., 1 H) 9.01 (d, J=4.29 Hz, 1 H) 8.45 (s, 1 H) 8.28 (s, 1 H) 8.14 (d, J=8.84 Hz, 1 H) 7.96 (s, 1 H) 7.91 (s, 1 H) 7.84-7.89 (m, 1 H) 7.64 (d, J=4.29 Hz, 1 H) 7.58 (d, J=8.08 Hz, 1 H)

### EXAMPLE 62

### Methyl 6-{(Z)-[2-[(2,6-dichlorophenyl)amino]-4-oxo-1,3-thiazol-5(4H)-ylidene]methyl}-4-quinolinecarboxylate trifluoroacetate

The title compound was made by following the method described in Example 61 as a yellow solid (46%). Methanol was used in place of ammonium hydroxide. 1H NMR (400 MHz, DMSO-d₆) δ ppm 13.11 (br. s.,1 H) 9.10 (d, J=4.29 Hz, 1 H) 8.72 (d, J=1.52 Hz, 1 H) 8.19 (d, J=8.84 Hz, 1 H) 8.01 (d, J=1.77 Hz, 1 H) 7.99 (d, J=4.29 Hz, 1 H) 7.94 (s, 1 H) 7.60 (d, J=8.08 Hz, 2 H) 7.25 (t, J=8.21 Hz, 1 H) 3.75 (s, 3 H)

### EXAMPLE 63

### 6-{(Z)-[2-[(2,6-Dichlorophenyl)amino]-4-oxo-1,3-thiazol-5(4H)-ylidene]methyl}-N-methyl-4-quinolinecarboxamide trifluoroacetate

The title compound was made by following the method described in Example 61 as a brown solid (26%). Methylamine was used in place of ammonia hydroxide. 1H NMR (400 MHz, DMSO-d₆) δ ppm 13.06 (br. s., 1 H) 8.99 (d, J=4.29 Hz, 1 H) 8.74 (d, J=4.80 Hz, 1 H) 8.20 (d, J=1.52 Hz, 1 H) 8.14 (d, J=8.84 Hz, 1 H) 7.96 (dd, J=8.84, 1.77 Hz, 1 H) 7.92 (s, 1 H) 7.54-7.63 (m, 3 H) 7.25 (t, J=8.21 Hz, 1 H) 2.62 (d, J=4.55 Hz, 3 H)

### EXAMPLE 64

### 6-{(Z)-[2-[(2,6-Dichlorophenyl)amino]-4-oxo-1,3-thiazol-5(4H)-ylidene]methyl}-N,N-dimethyl-4-quinolinecarboxamide trifluoroacetate

The title compound was made by following the method described in Example 61 as a yellow solid (47%). Dimethylamine was used in place of ammonia hydroxide. 1H NMR (400 MHz, DMSO-d₆) δ ppm 13.08 (br. s., 1 H) 9.00 (d, J=4.29 Hz, 1 H) 8.16 (d, J=8.84 Hz, 1 H) 7.94-8.02 (m, 2 H) 7.79 (d, J=1.52 Hz, 1 H) 7.60 (d, J=8.08 Hz, 2 H) 7.53 (d, J=4.29 Hz, 1 H) 7.27 (t, J=8.21 Hz, 1 H) 2.85 (s, 3 H) 2.62 (s, 3 H)

### EXAMPLE 65

### (5Z)-2-[(2,6-Dichlorophenyl)amino]-5-{[4-(4-morpholinylcarbonyl)-6-quinolinyl]methylidene}-1,3-thiazol-4(5H)-one trifluoroacetate

The title compound was made by following the method described in Example 61 as a grey solid (70%). Morpholine was used in place of ammonium hydroxide. MS(ES+) m/e 513 [M+H].

### EXAMPLE 66

### (5Z)-2-[(2,6-Dichlorophenyl)amino]-5-({4-[(4-methyl-1-piperazinyl)carbonyl]-6-quinolinyl}methylidene)-1,3-thiazol-4(5H)-one trifluoroacetate

The title compound was made by following the method described in Example 61 as a yellow solid (44%). 1-methylpiperazine was used in place of ammonium hydroxide. 1H NMR (400 MHz, DMSO-d₆) δ ppm 13.08 (br. s., 1 H) 9.97 (br. s., 1 H) 9.04 (d, J=4.04 Hz, 1 H) 8.18 (d, J=8.84 Hz, 1 H) 8.07 (d, J=1.52 Hz, 1 H) 8.00 (s, 1 H) 7.88 (dd, J=8.97, 1.64 Hz, 1 H) 7.55-7.67 (m, 3 H) 7.27 (t, J=8.08 Hz, 1 H) 4.60 (br. s., 2 H) 3.53-3.69 (m, 2 H) 3.08-3.31 (m, 4 H) 2.84 (s, 3 H)

### EXAMPLE 67

### (5Z)-2-[(2,6-Dichlorophenyl)amino]-5-{[3-(dimethylamino)-6-quinoxalinyl]methylidene}-1,3-thiazol-4(5H)-one

Following the procedures of Examples 1c), 1d), 1e), and 1f), except substituting dimethylamine for morpholine, the title compound was obtained as a yellow solid. ¹H NMR (400 MHz, CHLOROFORM-d) δ ppm 8.80 (s, 1 H) 8.53 (s, 1 H) 7.88 (d, J=7.0 Hz, 2 H) 7.73 (s, 1 H) 7.39 (d, J=8.1 Hz, 2 H) 7.34-7.39 (m, 1 H) 7.09 (t, J=8.1 Hz, 1 H) 3.29 (s, 6 H).

### EXAMPLE 68

### (5Z)-2-[(2,6-Dichlorophenyl)amino]-5-({3-[2-(dimethylamino)ethyl]-6-quinoxalinyl}methylidene)-1,3-thiazol-4(5H)-one

a) 2-(2-Chloroethyl)-7-ethenylquinoxaline. A solution of 7-bromo-2(1H-quinoxalinone (prepared by the method of Linda, P; Marino, G. Ric. Sci. Rend., Ser. A. 1963, 3, 225-228) (3.00 g; 13.3 mmol), vinylboronic anhydride pyridine complex (1.60 g; 6.67 mmol), potassium carbonate (1.84 g; 13.3 mmol), and tetrakis(triphenylphosphine)palladium (1.54 g; 1.33 mmol) in 1,2-dimethoxyethane (21.0 mL) and water (7.0 mL) was heated to 85° C. overnight. Upon cooling to ambient temperature, water was added and the reaction mixture was extracted thrice with ethyl acetate. The combined organic layers were dried over MgSO₄, filtered, and concentrated in vacuo. The residue was treated with phosphorus oxychloride (3.0 mL) and heated under reflux overnight. Upon cooling to ambient temperature, water was added, the solution was neutralized with solid NaHCO₃, and the reaction mixture was extracted twice with ethyl acetate. The combined organic layers were dried over MgSO₄, filtered, and concentrated in vacuo. Purification via flash column chromatography (silica gel, 0-20% ethyl acetate in hexanes) afforded a mixture of the title compound and 2-chloro-7-ethenylquinoxaline as a white solid. MS(ES+) m/e 219 [M+H]⁺.
b) [2-(7-Ethenyl-2-quinoxalinyl)ethyl]dimethylamine. Following the procedure of Example 1c), except substituting dimethylamine for morpholine and the compound from Example 68a) for the compound from Example 1b), the title compound was obtained as a white solid. ¹H NMR (400 MHz, CHLOROFORM-d) δ ppm 8.45 (s, 1 H) 7.78 (d, J=8.3 Hz, 1 H) 7.54 (s, 1 H) 7.22 (dd, J=8.5, 1.9 Hz, 1 H) 5.78-5.96 (m, 1 H) 5.02-5.11 (m, 1 H) 4.94-5.01 (m, 1 H) 3.28 (s, 6 H) 2.81-2.90 (m, 2 H) 2.38-2.53 (m, 2 H). MS(ES+) m/e 228 [M+H]⁺.
c) (5Z)-2-[(2,6-Dichlorophenyl)amino]-5-({3-[2-(dimethylamino)ethyl]-6-quinoxalinyl}methylidene)-1,3-thiazol-4(5H)-one. Following the procedures of Examples 1e) and 1f), except substituting the compound from Example 68b) for the compound from Example 1d), the title compound was obtained as a light yellow solid. ¹H NMR (400 MHz, DMSO-d₆) δ ppm 12.6 (s, 1 H) 8.61 (s, 1 H) 7.68 (d, J=8.3 Hz, 1 H) 7.49 (d, J=8.3 Hz, 2 H) 7.41 (s, 1 H) 7.19 (dd, J=6.7, 1.4 Hz, 1 H) 7.16 (d, J=8.1 Hz, 1 H) 6.79 (t, J=7.5 Hz, 1 H) 3.20 (s, 6 H) 2.90 (t, J=7.2 Hz, 2 H) 2.53 (t, J=7.2 Hz, 2 H). MS(ES+) m/e 472 [M+H]⁺.

### EXAMPLE 69

### Ethyl 6-{(Z)-[2-({2-chloro-5-[(2-methylpropanoyl)amino]phenyl}amino)-4-oxo-1,3-thiazol-5(4H)-ylidene]methyl}-4-(4-pyridinyl)-3-quinolinecarboxylate

a) N-(3-Amino-4-chlorophenyl)-2-methylpropanamide. Isobutyric anhydride (2.33 mL, 14.1 mmol) was injected into a stirred mixture of 4-chloro-1,3-phenylenediamine (2.00 g, 14.0 mmol) and dichloromethane (30 mL) and stirring continued for 2 h. Solvent was removed under reduced pressure until a precipitate had formed. The precipitate was filtered, washed with a little dichloromethane and dried to give the title compound (2.06 g, 69%) as a purple-white powder. 1H NMR (400 MHz, CHLOROFORM-d) δ ppm 1.26 (d, J=6.82 Hz, 6 H) 2.49 (septet, J=6.86 Hz, 1 H) 4.09 (s, 2 H) 6.61 (dd, J=8.59, 2.53 Hz, 1 H) 7.12 (s, 1 H) 7.16 (d, J=8.59 Hz, 1 H) 7.38 (d, J=2.27 Hz, 1 H)
b) N-{4-Chloro-3-[(4-oxo-4,5-dihydro-1,3-thiazol-2-yl)amino]phenyl}-2-methylpropanamide. A mixture of the compound from example 69(a) (2.06 g, 9.71 mmol), 2-(methylthio)-1,3-thiazol-4(5H)-one (1.43 g, 9.71 mmol) and ethanol (10 mL) was heated under reflux for 21 h, then cooled and diluted with dichloromethane (10 mL). The solid was filtered and dried to give the title compound (2.12 g, 70%) as a solid. 1H NMR (400 MHz, DMSO-d₆) δ ppm 1.09 (d, J=6.82 Hz, 6 H) 2.57 (septet, J=6.82 Hz, 1 H) 4.03 (s, 2 H) 7.30 (dd, J=8.84, 2.02 Hz, 1 H) 7.38 (d, J=8.59 Hz, 1 H) 7.45 (s, 1 H) 9.97 (s, 1 H) 11.99 (s, 1 H).
c) The title compound was made by following the method described in Example 57c) as a yellow solid (31%). The compound from example 69(b) was used in place of 2-[(2,6-dichlorophenyl)amino]-1,3-thiazol-4(5H)-one. 1H NMR (400 MHz, DMSO-d₆) δ ppm 12.72 (br. s., 1 H) 10.12 (s, 1 H) 9.32 (s, 1 H) 8.45 (d, J=5.81 Hz, 1 H) 8.24 (d, J=8.59 Hz, 1 H) 8.08 (dd, J=8.84, 1.77 Hz, 1 H) 7.82 (s, 1 H) 7.46-7.55 (m, 2 H) 7.32 (d, J=5.81 Hz, 1 H) 4.07 (q, J=7.16 Hz, 2 H) 2.58-2.71 (m, 1 H) 1.11 (d, J=6.82 Hz, 6 H) 0.92 (t, J=7.07 Hz, 3 H)

### EXAMPLE 70

### (5Z)-2-[(2,6-Dichlorophenyl)amino]-5-{[3-(methyloxy)-6-quinoxalinyl]methylidene}-1,3-thiazol-4(5H)-one

a) 2-Chloro-7-ethenylquinoxaline. A solution of the compound from Example 2a) (0.850 g; 4.94 mmol) in phosphorus oxychloride (5.0 mL) was heated under reflux overnight. Upon cooling to ambient temperature, water was added, the solution was neutralized with solid NaHCO₃, and the reaction mixture was extracted twice with ethyl acetate. The combined organic layers were dried over MgSO₄, filtered, and concentrated in vacuo. Purification via flash column chromatography (silica gel, 0-20% ethyl acetate in hexanes) afforded the title compound (0.169 g, 18%) as a white solid. ¹H NMR (400 MHz, CHLOROFORM-d) δ ppm 8.73 (s, 1 H) 8.06 (d, J=8.6 Hz, 1 H) 7.94 (d, J=1.8 Hz, 1 H) 7.91 (dd, J=8.6, 1.8 Hz, 1 H) 6.92 (dd, J=17.7, 10.9 Hz, 1 H) 6.01 (d, J=17.7 Hz, 1 H) 5.52 (d, J=10.9 Hz, 1 H).
b) 3-(Methyloxy)-6-quinoxalinecarbaldehyde. A suspension of the compound from Example 70a) (0.130 g; 0.682 mmol) and potassium carbonate (0.188 g; 1.36 mmol) in methanol (2.0 mL) was irradiated at 150° C. for 20 min. in a Biotage Initiator microwave synthesizer. Upon cooling, the reaction mixture was filtered and concentrated in vacuo. To a solution of the residue in dioxane (3.0 mL) and water (1.0 mL) was added osmium tetroxide (2.5% soln. in t-BuOH; 0.139 g; 0.014 mmol), sodium periodate (0.583 g; 2.73 mmol), and 2,6-lutidine (0.159 mL; 1.36 mmol). After stirring 30 min. at ambient temperature, water was added and the reaction mixture was extracted twice with CH₂Cl₂. The combined organic layers were dried over MgSO₄, filtered, and concentrated in vacuo. Purification via flash column chromatography (silica gel, 10-20% ethyl acetate in hexanes) afforded the title compound (0.109 g; 85%) as a white solid. ¹H NMR (400 MHz, CHLOROFORM-d) δ ppm 10.2 (s,1 H) 8.55 (s, 1 H) 8.32 (d, J=1.5 Hz, 1 H) 8.11 (d, J=8.7 Hz, 1 H) 8.04 (dd, J=8.4, 1.8 Hz, 1 H) 4.12 (s, 3 H).
c) (5Z-2-[(2,6-Dichlorophenyl)amino]-5-{[3-(methyloxy)-6-quinoxalinyl]methylidene}-1,3-thiazol-4(5H)-one. Following the procedure of Example 1f), except substituting the compound from Example 70b) for the compound from Example 1e), the title compound was obtained as a beige solid. ¹H NMR (400 MHz, DMSO-d₆) δ ppm 13.1 (s, 1 H) 8.63 (s, 1 H) 8.07 (d, J=8.6 Hz, 1 H) 7.94 (s,1 H) 7.91 (d, J=1.3 Hz, 1 H) 7.73 (dd, J=8.7, 1.4 Hz, 1 H) 7.59 (d, J=8.1 Hz, 2 H) 7.25 (t, J=8.2 Hz, 1 H) 4.03 (s, 3 H).

### EXAMPLE 71

### (5Z)-2-[(2,6-Dichlorophenyl)amino]-5-{[3-(4-methyl-1-piperazinyl)-6-quinoxalinyl]methylidene}-1,3-thiazol-4(5H)-one

a) A solution of the compound from Example 70a) (0.100 g; 0.525 mmol) and 1-methylpiperazine (0.116 mL; 1.05 mmol) in methanol (2.0 mL) was irradiated at 150° C. for 20 min. in a Biotage Initiator microwave synthesizer. Upon cooling, the reaction mixture was concentrated in vacuo. To a solution of the residue in dioxane (3.0 mL) and water (1.0 mL) was added osmium tetroxide (2.5% soln. in t-BuOH; 0.107 g; 0.011 mmol), sodium periodate (0.449 g; 2.10 mmol), and 2,6-lutidine (0.122 mL; 1.05 mmol). After stirring 30 min. at ambient temperature, water was added and the reaction mixture was extracted twice with CH₂Cl₂. The combined organic layers were dried over MgSO₄, filtered, and concentrated in vacuo. Purification via flash column chromatography (silica gel, 60-100% ethyl acetate in hexanes) afforded the title compound (0.086 g; 64%) as a yellow solid. MS(ES+) m/e 257 [M+H]⁺.
b) (5Z)-2-[(2,6-Dichlorophenyl)amino]-5-{[3-(4-methyl-1-piperazinyl)-6-quinoxalinyl]methylidene}-1,3-thiazol-4(5H)-one. Following the procedure of Example 1f), except substituting the compound from Example 71a) for the compound from Example 1e), the title compound was obtained as a yellow solid. ¹H NMR (400 MHz, MeOD) δ ppm 8.69 (s, 1 H) 7.83 (d, J=8.6 Hz, 1 H) 7.75 (s, 1 H) 7.67 (s, 1 H) 7.48 (dd, J=8.7, 1.6 Hz, 1 H) 7.44 (d, J=8.1 Hz, 2 H) 7.15 (t, J=8.1 Hz, 1 H) 3.81-3.88 (m, J=3.8 Hz, 4 H) 2.58-2.65 (m, 4 H) 2.38 (s, 3 H).

### EXAMPLE 72

### (5Z)-2-[(2,6-Dichlorophenyl)amino]-5-[(3-methyl-6-quinoxalinyl)methylidene]-1,3-thiazol-4(5H)-one

a) 7-Ethenyl-2-quinoxalinyl trifluoromethanesulfonate. To a solution of the compound from Example 2a) (0.260 g; 1.51 mmol) in CH₂Cl₂ (7.0 mL) was added trifluoromethanesulfonic anhydride (0.254 mL; 1.51 mmol) and triethylamine (0.232 mL; 1.66 mmol). After stirring 30 min. at ambient temperature, the solution was concentrated onto silica gel and purified via flash column chromatography (silica gel, 0-20% ethyl acetate in hexanes) to afford the title compound (0.409 g; 89%) as a white solid. ¹H NMR (400 MHz, CHLOROFORM-d) δ ppm 8.71 (s, 1 H) 8.14 (d, J=9.3 Hz, 1 H) 7.99 (dd, J=6.5, 1.8 Hz, 1 H) 7.98 (s, 1 H) 6.93 (dd, J=17.6, 11.0 Hz, 1 H) 6.05 (d, J=17.4 Hz, 1 H) 5.56 (d, J=10.9 Hz, 1 H).
b) 7-Ethenyl-2-methylquinoxaline. A solution of the compound from Example 72a) (0.200 g; 0.657 mmol), trimethylboroxine (0.091 mL; 0.657 mmol), potassium carbonate (0.273 g; 1.972 mmol), and tetrakis(triphenylphosphine)palladium (0.076 g; 0.066 mmol) in dioxane (2.0 mL) was irradiated at 150° C. for 30 min. in a Biotage Initiator microwave synthesizer. Upon cooling, the solution was filtered through celite which was subsequently washed with CH₂Cl₂. Purification via flash column chromatography (silica gel, 10-30% ethyl acetate in hexanes) afforded the title compound (0.073 g; 65%) as a yellow solid. MS(ES+) m/e 171 [M+H]⁺.
c) (5Z)-2-[(2,6-Dichlorophenyl)amino]-5-[(3-methyl-6-quinoxalinyl)methylidene]-1,3-thiazol-4(5H)-one. Following the procedures of Examples 1e) and 1f), except substituting the compound from Example 72b) for the compound from Example 1d), the title compound was obtained as a light orange solid. ¹H NMR (400 MHz, DMSO-d₆) δ ppm 13.1 (s, 1 H) 8.87 (s, 1 H) 8.11 (d, J=8.6 Hz, 1 H) 8.05 (d, J=1.5 Hz, 1 H) 7.96 (s, 1 H) 7.87 (dd, J=8.7, 1.9 Hz, 1 H) 7.58 (d, J=8.1 Hz, 2 H) 7.25 (t, J=8.2 Hz, 1 H) 2.70 (s, 3 H).

### EXAMPLE 73

### (5Z)-2-[(2,6-Dichlorophenyl)amino]-5-[(3-phenyl-6-quinoxalinyl)methylidene]-1,3-thiazol-4(5H)-one

Following the procedures of Examples 72b) and 72c), except substituting triphenylboroxine for trimethylboroxine, the title compound was obtained as a yellow solid. ¹H NMR (400 MHz, DMSO-d₆) δ ppm 13.1 (s, 1 H) 9.60 (s,1 H) 8.27-8.40 (m, 3 H) 8.15-8.23 (m, 2 H) 7.96 (m, 2 H) 7.60 (m, 4 H) 7.23 (t, J=8.2 Hz, 1 H).

### EXAMPLE 74

### (5Z)-2-[(2,6-Dichlorophenyl)amino]-5-({3-[(2-hydroxyethyl)(methyl)amino]-6-quinoxalinyl}methylidene)-1,3-thiazol-4(5H)-one

Following the procedures of Examples 1c), 1d), 1e), and 1f), except substituting 2-(methylamino)ethanol for morpholine, the title compound was obtained as a yellow solid. ¹H NMR (400 MHz, DMSO-d₆) δ ppm 13.0 (s, 1 H) 8.73 (s, 1 H) 7.85 (s, 1 H) 7.84 (s, J=8.6 Hz, 1 H) 7.63 (d, J=1.0 Hz, 1 H) 7.58 (d, J=8.1 Hz, 2 H) 7.41 (dd, J=8.6, 1.0 Hz, 1 H) 7.24 (t, J=8.1 Hz, 1 H) 4.81 (s, 1 H) 3.75 (t, J=5.2 Hz, 2 H) 3.64 (t, J=4.5 Hz, 2 H) 3.19 (s, 3 H).

### EXAMPLE 75

### (5Z)-2-[(2,6-Dichlorophenyl)amino]-5-({3-[[2-(dimethylamino)ethyl](methyl)amino]-6-quinoxalinyl}methylidene)-1,3-thiazol-4(5H)-one

a) N-(7-Ethenyl-2-quinoxalinyl)-N,N′,N′-trimethyl-1,2-ethanediamine. A solution of the compound from Example 1b) (0.300 g; 1.23 mmol) and N,N′,N′-trimethylethylenediamine (0.176 mL; 1.36 mmol) in dioxane (2.0 mL) was irradiated at 150° C. for 20 min. in a Biotage Initiator microwave synthesizer. Upon cooling, tributyl(vinyl)tin (0.395 mL; 1.36 mmol), N,N-dimethylformamide (3 drops), and tetrakis(triphenylphosphine)palladium (0.014 g; 0.012 mmol) were added to the reaction mixture, which was further irradiated at 150° C. for 20 min. in a Biotage Initiator microwave synthesizer. Upon cooling, the solution was concentrated onto silica gel and purified via flash column chromatography (silica gel, 0-10% methanol in CH₂Cl₂) to afford the title compound (0.306 g; 97%) as a light yellow solid. ¹H NMR (400 MHz, CHLOROFORM-d) δ ppm 8.39 (s, 1 H) 7.76 (d, J=8.3 Hz, 1 H) 7.57 (d, J=1.8 Hz, 1 H) 7.44 (dd, J=8.5, 1.9 Hz, 1 H) 6.80 (dd, J=17.6, 11.0 Hz, 1 H) 5.86 (d, J=1 6.9 Hz, 1 H) 5.33 (d, J=11.4 Hz, 1 H) 3.83 (t, J=7.2 Hz, 2 H) 3.23 (s, 3 H) 2.67 (t, J=7.2 Hz, 2 H) 2.39 (s, 6 H).
b) (5Z)-2-[(2,6-Dichlorophenyl)amino]-5-({3-[[2-(dimethylamino)ethyl](methyl)amino]-6-quinoxalinyl}methylidene)-1,3-thiazol-4(5H)-one. Following the procedures of Examples 1e) and 1f), except substituting the compound from Example 75a) for the compound from Example 1d), the title compound was obtained as an orange solid. ¹H NMR (400 MHz, DMSO-d₆) δ ppm 8.67 (s, 1 H) 7.83 (d, J=8.6 Hz, 1 H) 7.59-7.73 (m, 2 H) 7.46-7.56 (m, J=9.2, 6.2 Hz, 2 H) 7.39-7.45 (m, 1 H) 7.16 (t, J=8.2 Hz, 1 H) 3.78-3.89 (m, 2 H) 3.34 (s, 6 H) 3 H) 2.71 (t, J=7.6 Hz, 2 H).

### EXAMPLE 76

### (5Z)-2-[(2,6-Dichlorophenyl)amino]-5-{[3-(phenylamino)-6-quinoxalinyl]methylidene}-1,3-thiazol-4(5H)-one

Following the procedures of Examples 75a) and 75b), except substituting aniline for N,N′,N′-trimethylethylenediamine, the title compound was obtained as a light orange solid. ¹H NMR (400 MHz, DMSO-d₆) δ ppm 13.0 (s, 1 H) 10.1 (s, 1 H) 8.57 (s, 1 H) 7.93 (d, J=8.6 Hz, 2 H) 7.90 (s, 1 H) 7.86 (s, 1 H) 7.58 (d, J=8.1 Hz, 2 H) 7.50 (dd, J=8.1, 1.3 Hz, 1 H) 7.37 (t, J=7.7 Hz, 3 H) 7.25 (t, J=8.0 Hz, 1 H) 7.06 (t, J=7.3 Hz, 1 H).

### EXAMPLE 77

### N-{4-Chloro-3-[((5Z)-5-{[3-(4-morpholinyl)-6-quinoxalinyl]methylidene}-4-oxo-4,5-dihydro-1,3-thiazol-2-yl)amino]phenyl}cyclobutanecarboxamide

Following the procedure of Example 1f), except substituting N-{4-chloro-3-[(4-oxo-4,5-dihydro-1,3-thiazol-2-yl)amino]phenyl}cyclobutanecarboxamide (example 47b) for the compound from Example 1(a), the title compound was obtained as a light orange solid. ¹H NMR (400 MHz, DMSO-d₆) δ ppm 12.8 (s, 1 H) 9.93 (s, 1 H) 8.84 (s,1 H) 7.89 (d, J=8.6 Hz, 1 H) 7.82 (s, 1 H) 7.68 (d, J=1.5 Hz, 1 H) 7.52 (dd, J=8.6, 1.8 Hz, 1 H) 7.47 (s, 1 H) 7.45 (s, 1 H) 7.41 (dd, J=9.0, 2.1 Hz, 1 H) 3.73 (s, 8 H) 3.12-3.26 (m, 1 H) 2.14-2.25 (m, 2 H) 2.03-2.14 (m, 2 H) 1.86-1.99 (m, 1 H) 1.72-1.84 (m, 1 H).

### EXAMPLE 78

### (5Z)-5-[(3-Amino-6-quinoxalinyl)methylidene]-2-[(2,6-dichlorophenyl)amino]-1,3-thiazol-4(5H)-one

a) 8-Bromotetrazolo[1,5-a]quinoxaline. A solution of the compound from Example 1b) (0.430 g; 1.77 mmol) and sodium azide (0.138 g; 2.12 mmol) in N,N-dimethylformamide (5.0 mL) was heated to 120° C. for 2 h. Upon cooling to ambient temperature, water was added and the resulting precipitate was collected by filtration, washed with water, and dried in vacuo to afford the title compound (0.403 g; 91%) as a light orange solid. ¹H NMR (400 MHz, CHLOROFORM-d) δ ppm 9.58 (s, 1 H) 8.84 (d, J=2.0 Hz, 1 H) 8.20 (d, J=8.8 Hz, 1 H) 7.99 (dd, J=8.7, 2.1 Hz, 1 H).
b) 8-Ethenyltetrazolo[1,5-a]quinoxaline. Following the procedure of Example 1d), except substituting the compound from Example 78a) for the compound from Example 1c), the title compound was obtained as an off-white solid. ¹H NMR (400 MHz, CHLOROFORM-d) δ ppm 9.51 (s, 1 H) 8.60 (d, J=1.8 Hz, 1 H) 8.25 (d, J=8.6 Hz, 1 H) 7.92 (dd, J=8.6, 1.8 Hz, 1 H) 6.97 (dd, J=17.6, 11.0 Hz, 1 H) 6.12 (d, J=17.4 Hz, 1 H) 5.63 (d, J=10.9 Hz, 1 H).
c) Tetrazolo[1,5-a]quinoxaline-8-carbaldehyde. Following the procedure of Example 1e), except substituting the compound from Example 78b) for the compound from Example 1d), the title compound was obtained as a light yellow solid. ¹H NMR (400 MHz, CHLOROFORM-d) δ ppm 10.3 (s, 1 H) 9.70 (s, 1 H) 9.14 (d, J=1.5 Hz, 1 H) 8.51 (d, J=8.6 Hz, 1 H) 8.38 (dd, J=8.5, 1.6 Hz, 1 H).
d) (5Z)-5-[(3-Amino-6-quinoxalinyl)methylidene]-2-[(2,6-dichlorophenyl)amino]-1,3-thiazol-4(5H)-one. Following the procedure of Example 1f), except substituting the compound from Example 78c) for the compound from Example 1e), the title compound was obtained following purification via flash column chromatography (silica gel, 10-100% ethyl acetate in hexanes) as a light orange solid. ¹H NMR (400 MHz, DMSO-d₆) δ ppm 13.0 (s, 1 H) 8.29 (s, 1 H) 7.83 (s, 1 H) 7.81 (d, J=8.8 Hz, 1 H) 7.59 (d, J=8.3 Hz, 2 H) 7.55 (s,1 H) 7.44 (dd, J=8.5, 0.6 Hz, 1 H) 7.25 (t, J=8.2 Hz, 1 H) 7.17 (s, 2 H). MS(ES+) m/e 416 [M+H]⁺.

### EXAMPLE 79

### N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}-2-methylpropanamide trifluoroacetate.

The title compound was made by following the method described in Example 49 as a yellow solid (35%). The compound from example 69(b) was used in place of 2-[(2,6-dichlorophenyl)amino]-1,3-thiazol-4(5H)-one. 1H NMR (400 MHz, DMSO-d₆) δ ppm 12.85 (br. s., 1 H) 10.21 (br. s., 1 H) 10.08 (s, 1 H) 8.83 (d, J=5.81 Hz, 1 H) 8.27 (s, 1 H) 8.11 (d, J=8.84 Hz, 1 H) 8.04 (s, 1 H) 7.95 (dd, J=8.84, 1.26 Hz, 1 H) 7.45-7.54 (m, 1 H) 7.40 (dd, J=8.84, 2.53 Hz, 1 H) 7.29 (d, J=6.06 Hz, 1 H) 4.03 (br. s., 3 H) 3.14-3.65 (m, 5 H) 2.87 (s, 3 H) 2.52-2.62 (m, 1 H) 1.08 (d, J=6.82 Hz, 6 H)

### EXAMPLE 80

### N-{4-Chloro-3-[((5Z)-5-{[4-(4-morpholinyl)-6-quinolinyl]methylidene}-4-oxo-4,5-dihydro-1,3-thiazol-2-yl)amino]phenyl}-2-methylpropanamide trifluoroacetate

The title compound was made by following the method described in example 79 as a orange solid (20%). Morpholine was used in place of 1-methylpiperazine. 1H NMR (400 MHz, DMSO-d₆) δ ppm 12.87 (br. s., 1 H) 10.06 (s, 1 H) 8.73 (d, J=6.57 Hz, 1 H) 8.17 (s, 1 H) 8.02-8.10 (m, 2 H) 7.95 (s, 1 H) 7.59 (d, J=2.02 Hz, 1 H) 7.45-7.49 (m, 1 H) 7.37 (dd, J=8.84, 2.27 Hz, 1 H) 7.22 (d, J=6.82 Hz, 1 H) 3.67 (br. s., 8 H) 2.54-2.63 (m, 1 H) 1.09 (d, J=6.82 Hz, 6 H)

### EXAMPLE 81

### N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}ethanesulfonamide trifluoroacetate

a) 1,1-Dimethylethyl (3-amino-4-chlorophenyl)carbamate. A solution of di-t-butyl dicarbonate (3.37 g, 15.4 mmol) in dichloromethane (8 mL) was added over 5 min to a stirred suspension of 2-chloro-1,3-benzenediamine (2.00 g, 14.0 mmol) in dichloromethane (22 mL) at room temperature. After stirring 48 h, the solvent was removed under reduced pressure and the residue slurried in dichloromethane (15 mL), then filtered, washed with 20% dichloromethane/hexane and dried. The filtrate was evaporated to dryness under reduced pressure and a second crop of product obtained by recrystallisation from 2:1 hexane:ether. Both crops were combined to give the title compound (2.83 g, 83%) as a solid. 1H NMR (400 MHz, DMSO-d₆) δ 1.46 (s, 9H), 5.25 (br s, 2H), 6.56 (dd, J=8.7, 2.4 Hz, 1 H), 7.01 (d, J=8.6 Hz, 1 H), 7.08 (d, J=2.0 Hz, 1H), 9.18 (s, 1H).
b) 1,1-Dimethylethyl {4-chloro-3-[(4-oxo-4,5-dihydro-1,3-thiazol-2-yl)amino]phenyl}carbamate. A mixture of the compound from example 31(a) (1.75 g, 7.21 mmol), 2-(methylthio)-1,3-thiazol-4(5H)-one (1.27 g, 8.63 mmol) and ethanol (25 mL) was heated under reflux for 24 h, then cooled. The solid was filtered, washed with ethanol and dried to give the title compound (1.38 g, 56%) as a solid. 1H NMR (400 MHz, DMSO-d₆) δ 1.47 (s, 9H), 4.03 (s, 2H), 7.14-7.25 (m, 2H), 7.33 (d, J=8.6, 1 H), 9.51 (s, 1 H), 11.97 (br s,1 H).
c) 2-[(5-Amino-2-chlorophenyl)amino]-1,3-thiazol-4(5H)-one. A solution of the compound from example 31(b) (0.200 g, 0.585 mmol) in trifluoroacetic acid (3 mL) was stirred at room temperature for 20 min, then evaporated under reduced pressure. The residue was dissolved in water (20 mL), filtered, then adjusted to pH 7-8 with aq NaOH. The precipitate was filtered after 2 h, washed with water and dried to leave the title compound (0.105 g, 74%) as a pale orange powder. 1H NMR (400 MHz, DMSO-d₆) δ ppm 3.99 (s, 2 H) 5.27 (s, 2 H) 6.19 (s, 1 H) 6.33 (d, J=6.57 Hz, 1 H) 7.05 (d, J=8.59 Hz, 1 H) 11.84 (s, 1 H).
d) N-{4-Chloro-3-[(4-oxo-4,5-dihydro-1,3-thiazol-2-yl)amino]phenyl}ethanesulfonamide. A solution of ethanesulfonyl chloride (0.147 mL, 1.55 mmol) in dioxane (1 mL) was injected into a stirred mixture of the compound from example 81(c) (0.250 g, 0.830 mmol), 1,4-diazabicyclo[2.2.2]octane (0.060 g, 0.540 mmol) and dioxane (1 mL) and stirring continued for 1 h. The mixture was diluted with water (20 mL) and extracted with ethyl acetate. The extracts were dried (MgSO₄) and evaporated under reduced pressure. The residue was chromatographed (silica gel, 0-10% methanol/chloroform) to give the title compound (0.298 g, 87%) as a solid. 1H NMR (400 MHz, DMSO-d₆) δ ppm 1.19 (t, J=7.33 Hz, 3 H) 3.12 (q, J=7.33 Hz, 2 H) 4.04 (s, 2 H) 6.89 (d, J=2.27 Hz, 1 H) 6.97 (dd, J=8.72, 2.40 Hz, 1 H) 7.43 (d, J=8.59 Hz, 1 H) 10.01 (s, 1 H) 12.01 (s, 1 H).
e) The title compound was made by following the method described in Example 49 as a brown solid (24%). The compound from example 81(d) was used in place of 2-(2,6-dichlorophenyl)amino]-1,3-thiazol-4(5H)-one. 1H NMR (400 MHz, DMSO-d₆) δ ppm 12.88 (br. s., 1 H) 10.23 (br. s., 1 H) 10.08 (s, 1 H) 8.85 (d, J=6.06 Hz, 1 H) 8.32 (s, 1 H) 8.12 (d, J=8.84 Hz, 1 H) 8.05 (s, 1 H) 7.97 (d, J=9.09 Hz, 1 H) 7.53 (d, J=8.59 Hz, 1 H) 7.33 (d, J=6.32 Hz, 1 H) 7.05 (dd, J=8.59, 2.53 Hz, 1 H) 6.98 (d, J=2.53 Hz, 1 H) 3.53 (br. s., 8 H) 3.18 (q, J=7.33 Hz, 2 H) 2.90 (s, 3 H) 1.20 (t, J=7.33 Hz, 3 H)

### EXAMPLE 82

### N-{4-Chloro-3-[((5Z)-5-{[4-(4-morpholinyl)-6-quinolinyl]methylidene}-4-oxo-4,5-dihydro-1,3-thiazol-2-yl)amino]phenyl}ethanesulfonamide trifluoroacetate

The title compound was made by following the method described in example 81 as a orange solid (17%). Morpholine was used in place of 1-methylpiperazine. 1H NMR (400 MHz, DMSO-d₆) δ ppm 12.88 (br. s., 1 H) 10.08 (s, 1 H) 8.73 (d, J=6.82 Hz, 1 H) 8.20 (s, 1 H) 8.06 (s, 2 H) 7.97 (s, 1 H) 7.51 (d, J=8.84 Hz, 1 H) 7.24 (d, J=6.82 Hz, 1 H) 7.05 (dd, J=8.72, 2.65 Hz, 1 H) 6.98 (d, J=2.53 Hz, 1 H) 3.71 (s, 8 H) 3.17 (q, J=7.33 Hz, 2 H) 1.20 (t, J=7.33 Hz, 3 H)

### EXAMPLE 83

### Ethyl 6-{(Z)-[2-({2-chloro-5-[(2-methylpropanoyl)amino]phenyl}amino)-4-oxo-1,3-thiazol-5(4H)-ylidene]methyl}-4-(4-morpholinyl)-3-quinolinecarboxylate trifluoroacetate

The title compound was made by following the method described in Example 56 as a orange solid (11%). The compound from example 69(b) was used in place of the compound from example 47(b). 1H NMR (400 MHz, DMSO-d₆) δ ppm 12.83 (br. s., 1 H) 10.02 (s, 1 H) 8.84 (s, 1 H) 8.24 (d, J=1.77 Hz, 1 H) 8.05-8.10 (m, 1 H) 7.99-8.05 (m, 1 H) 7.92 (s, 1 H) 7.60 (d, J=2.02 Hz, 1 H) 7.42-7.46 (m, 1 H) 7.36 (dd, J=8.00, 4.00 Hz, 1 H) 4.40 (q, J=6.99 Hz, 2 H) 3.51-3.60 (m, 4 H) 3.12-3.22 (m, 4 H) 1.36 (t, J=7.07 Hz, 3 H) 1.07 (d, J=6.57 Hz, 6 H)

### EXAMPLE 84

### Ethyl 6-{(Z)-[2-({2-chloro-5-[(2-methylpropanoyl)amino]phenyl}amino)-4-oxo-1,3-thiazol-5(4H)-ylidene]methyl}-3-quinolinecarboxylate trifluoroacetate

The title compound was made by following the method described in Example 28 as a brown solid (5%). The compound from example 69(b) was used in place of 2-[(2,6-dichlorophenyl)amino]-1,3-thiazol-4(5H)-one. 1H NMR (400 MHz, DMSO-d₆) δ ppm 12.79 (br. s., 1 H) 10.03 (s, 1 H) 9.32 (d, J=2.27 Hz, 1 H) 9.09 (d, J=1.77 Hz, 1 H) 8.37 (d, J=1.52 Hz, 1 H) 8.16 (d, J=8.84 Hz, 1 H) 8.00 (dd, J=8.72, 2.15 Hz, 1 H) 7.85 (s, 1 H) 7.39-7.51 (m, 3 H) 4.41 (q, J=7.07 Hz, 2 H) 2.52-2.63 (m, 1 H) 1.38 (t, J=7.20 Hz, 3 H) 1.09 (d, J=6.82 Hz, 6 H)

### EXAMPLE 85

### Ethyl 6-{(Z)-[2-({2-chloro-5-[(2-methylpropanoyl)amino]phenyl}amino)-4-oxo-1,3-thiazol-5(4H)-ylidene]methyl}-4-(4-methyl-1-piperazinyl)-3-quinolinecarboxylate trifluoroacetate

The title compound was made by following the method described in Example 45 as a yellow solid (5%). The compound from example 69(b) was used in place of 2-[(2,6-dichlorophenyl)amino]-1,3-thiazol-4(5H)-one. 1H NMR (400 MHz, DMSO-d₆) δ ppm 12.84 (br. s., 1 H) 10.05 (s, 1 H) 9.85 (br. s., 1 H) 8.90 (s, 1 H) 8.32 (s, 1 H) 8.12 (d, J=8.59 Hz, 1 H) 8.04 (s, 1 H) 7.91 (dd, J=8.84, 1.52 Hz, 1 H) 7.50 (d, J=9.09 Hz, 2 H) 7.40 (dd, J=12.00, 4.00 Hz, 1 H) 4.41 (q, J=7.07 Hz, 2 H) 3.34-3.49 (m, 8 H) 2.86 (s, 3 H) 2.54-2.62 (m, 1 H) 1.39 (t, J=7.07 Hz, 3 H) 1.08 (d, J=6.82 Hz, 6 H)

### EXAMPLE 86

### Ethyl 6-{(Z)-[2-({2-chloro-5-[(cyclobutylcarbonyl)amino]phenyl}amino)-4-oxo-1,3-thiazol-5(4H)-ylidene]methyl}-3-quinolinecarboxylate trifluoroacetate

The title compound was made by following the method described in Example 28 as a brown solid (21%). The compound from example 47(b) was used in place of 2-[(2,6-dichlorophenyl)amino]-1,3-thiazol-4(5H)-one. 1H NMR (400 MHz, DMSO-d₆) δ ppm 12.80 (br. s., 1 H) 9.93 (s, 1 H) 9.32 (d, J=1.77 Hz, 1 H) 9.11 (s, 1 H) 8.38 (d, J=1.52 Hz, 1 H) 8.17 (d, J=8.84 Hz, 1 H) 7.98-8.03 (m, 1 H) 7.86 (s, 1 H) 7.42-7.50 (m, 2 H) 4.42 (q, J=6.91 Hz, 2 H) 2.14-2.28 (m, 2 H) 2.02-2.14 (m, J=8.34, 8.34, 8.34 Hz, 2 H) 1.86-1.97 (m, 1 H) 1.70-1.84 (m, 1 H) 1.38 (t, J=7.07 Hz, 3 H)

### EXAMPLE 87

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| (5Z)-2-[(2,6-Dichlorophenyl)amino]-5-{[3-(4-morpholinyl)- | 25 mg |
| 6-quinoxalinyl]methylidene}-1,3-thiazol-4(5H)-one | |
| Lactose | 55 mg |
| Talc | 16 mg |
| Magnesium Stearate | 4 mg |

### EXAMPLE 88

### Injectable Parenteral Composition

An injectable form for administering the present invention is produced by stirring 1.5% by weight of 7-{(Z)-[2-[(2,6-Dichlorophenyl)amino]-4-oxo-1,3-thiazol-5(4H)-ylidene]methyl}-2(1 H)-quinoxalinone in 10% by volume propylene glycol in water.

### EXAMPLE 89

### Tablet Composition

The sucrose, calcium sulfate dihydrate and an hYAK inhibitor as shown in Table II below, are mixed and granulated in the proportions shown with a 10% gelatin solution. The wet granules are screened, dried, mixed with the starch, talc and stearic acid;, screened and compressed into a tablet.

TABLE II

| INGREDIENTS | AMOUNTS |
|---|---|
| (5Z)-2-[(2,6-Dichlorophenyl)amino]-5-{[2-(methylamino)- | 20 mg |
| 6-quinolinyl]methylidene}-1,3-thiazol-4(5H)-one | |
| calcium sulfate dehydrate | 30 mg |
| Sucrose | 4 mg |
| starch | 2 mg |
| talc | 1 mg |
| stearic acid | 0.5 mg |

### Biological Methods and Data

Because the compounds of the present invention are active as inhibitors of hYAK3 they exhibit therapeutic utility in treating diseases associated with hYAK3 activity, including but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia.

Substrate phosphorylation assays were carried out as follows:

### YAK3 Scintillation Proximity Assays Using Ser164 of Myelin Basic Protein as the Phosphoacceptor

The source of Ser164 substrate peptide The biotinylated Ser164, S164A peptide(LGGRDSRAGS*PMARRKK-ahx-Biotin-Amide), sequence derived from the C-terminus of bovine myelin basic protein (MBP) with Ser162 substituted as Ala 162, was purchased from California Peptide Research Inc. (Napa, Calif.), and its purity was determined by HPLC. Phosphorylation occurs at position 164 (marked S* above). The calculated molecular mass of the peptide is 2166 dalton. Solid sample was dissolved at 10 mM in DMSO, aliquoted, and stored at −20 C until use.

The source of enzyme:

hYAK3: Glutathione-S-Transferase (GST)-hYak3-His6 containing amino acid residues 142-526 of human YAK3 (aa 142-526 of SEQ ID NO 2. in U.S. Pat. No. 6,323,318) was purified from baculovirus expression system in Sf9 cells using Glutathione Sepharose 4B column chromatography followed by Ni-NTA-Agarose column chromatography. Purity greater than 65% typically is achieved. Samples, in 50 mM Tris, 150 mM NaCl, 10% glycerol, 0.1% Triton, 250 mM imidazole, 10 mM-mercapto ethanol, pH 8.0 were stored at −80 C until use.

Kinase assay of purified hYAK3: Assays were performed in 96 well (Costar, Catalog No.3789) or 384 well plates (Costar, Catalog No.3705). Reaction (in 10, 20, 25, or 40 μl volume) mix contained in final concentrations 25 mM Hepes buffer, pH 7.4; 10 mM MgCl₂; 10 mM-mercapto ethanol; 0.0025% Tween-20; 1 μM ATP, 0.1 μCi of [γ-³³P]ATP; purified hYAK3 (3.6-14 ng/assay; 4 nM final); and 4 μM Ser164 peptide. Compounds, titrated in DMSO, were evaluated at concentrations ranging from 50 μM to 0.2 nM. Final assay concentrations of DMSO did not exceed 5%, resulting in less than 15% loss of YAK3 activity relative to controls without DMSO. Reactions were incubated for 2 hours at room temperature and were stopped by addition of Streptavidin Scintillation Proximity beads (Amersham Pharmacia Biotech, Catalog No. RPNQ 0007 or Amersham Biosciences Catalog no. RPQ0626) in PBS, pH 7.4, 150 mM EDTA, and 0.1% Triton X-100. Under the assay conditions defined above, the Km(apparent) for ATP is determined to be 7.2+/−2.4 μM.

The data for compound dose responses were plotted as % Inhibition, calculated with the data reduction formula 100*(1-[(U1-C2)/(C1-C2)]), versus concentration of compound, where U is the unknown value, C1 is the average control value obtained for DMSO, and C2 is the average control value obtained for 0.05M EDTA. Data were fitted to the curve described by: y=((Vmax* x)/(K+x)) where Vmax is the upper asymptote and K is the IC50. The results for each compound were recorded as pIC50 calculated as follows: pIC50=−Log 10(K).

The compound of Example 1 was tested in the above assay and has pIC₅₀=8.9. The compounds of Example 2-5 were tested in the above assays and have pIC₅₀>7.

Utility of the Present Invention

The compounds of Formula I or II are useful for treating or preventing disease states in which hYAK3 proteins are implicated, especially diseases of the erythroid and hematopoietic systems, including but not limited to, anemias due to renal insufficiency or to chronic disease, such as autoimmunity, HIV, or cancer, and drug-induced anemias, myelodysplastic syndrome, aplastic anemia, myelosuppression, and cytopenia.

The compounds of Formula I or II are useful in treating diseases of the hematopoietic system, particularly anemias. Such anemias include an anemia selected from the group comprising: aplastic anemia and myelodysplastic syndrome. Such anemias also include those wherein the anemia is a consequence of a primary disease selected from the group consisting of: cancer, leukemia and lymphoma. Such anemias also include those wherein the anemia is a consequence of a primary disease selected from the group consisting of: renal disease, failure or damage. Such anemias include those wherein the anemia is a consequence of chemotherapy or radiation therapy, in particular wherein the chemotherapy is chemotherapy for cancer or AZT treatment for HIV infection. Such anemias include those wherein the anemia is a consequence of a bone marrow transplant or a stem cell transplant. Such anemias also include anemia of newborn infants. Such anemias also include those which are a consequence of viral, fungal, microbial or parasitic infection.

The compounds of Formula I or II are also useful for enhancing normal red blood cell numbers. Such enhancement is desirable for a variety of purposes, especially medical purposes such as preparation of a patient for transfusion and preparation of a patient for surgery.

What is claimed is:

1. A compound of Formula (I): 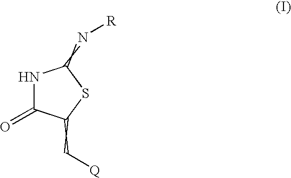
in which
R is selected form: aryl and substituted aryl; and
Q is 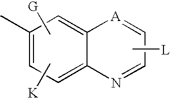
wherein
A is selected from CR⁵⁰ and N,
where R⁵⁰, G, K and L are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, —C(O)OR¹⁰, —C(O)NR¹¹R¹², oxo and cyano,
where, R¹⁰ is selected form hydrogen, C₁-C₄alkyl, aryl and trifluoromethyl, and R¹¹ and R¹² are independently selected form hydrogen, C₁-C₄alkyl, aryl and trifluoromethyl, provided that at least one of G, K, L and R⁵⁰, when R⁵⁰ is present, is not hydrogen.

2. A pharmaceutically acceptable salt, hydrate, solvate or pro-drug of a compound of Formula (I), as described in claim 1.

3. A compound according to claim 1 wherein A is nitrogen.

4. A pharmaceutically acceptable salt, hydrate, solvate or pro-drug of a compound of Formula (I), as described in claim 3.

5. A compound of claim 1 represented by the following Formula (II): 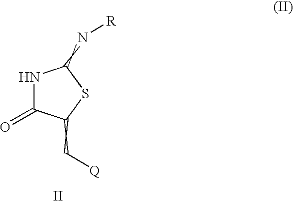
in which
R is selected form: C₁-C₁₂aryl and substituted C₁-C₁₂aryl; and
Q is 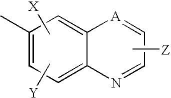
wherein
A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
where R¹⁰ is selected form hydrogen, C₁-C₄alkyl, aryl and trifluoromethyl, and R¹¹ and R¹² are independently selected form hydrogen, C₁-C₄alkyl, aryl and trifluoromethyl,
provided that at least one of X, Y, Z and R⁵¹, when R⁵¹ is present, is not hydrogen.

6. A pharmaceutically acceptable salt, hydrate, solvate or pro-drug of a compound of Formula (II), as described in claim 5.

7. A compound according to claim 5 wherein A is nitrogen.

8. A pharmaceutically acceptable salt, hydrate, solvate or pro-drug of a compound of Formula (II), as described in claim 7.

9. A compound of claim 1 selected from:
(5Z)-2-[(2,6-Dichlorophenyl)amino]-5-{[3-(4-morpholinyl)-6-quinoxalinyl]methylidene}-1,3-thiazol-4(5H)-one;
7-{(Z)-[2-[(2,6-Dichlorophenyl)amino]-4-oxo-1,3-thiazol-5(4H)-ylidene]methyl}-2(1H)-quinoxalinone;
(5Z)-2-[(2,6-Dichlorophenyl)amino]-5-{[2-(methylamino)-6-quinolinyl]methylidene}-1,3-thiazol-4(5H)-one;
(5Z)-2-[(2,6-Dichlorophenyl)amino]-5-{[4-(dimethylamino)-6-quinolinyl]methylidene}-1,3-thiazol-4(5H)-one;
(5Z)-2-[(2,6-Dichlorophenyl)amino]-5-{[4-(4-methyl-1-piperazinyl)-6-quinolinyl]methylidene}-1,3-thiazol-4(5H)-one;
(5Z)-5-[(2-Chloro-6-quinolinyl)methylidene]-2-[(2,6-dichlorophenyl)amino]-1,3-thiazol-4(5H)-one;
(5Z)-2-[(2,6-dichlorophenyl)amino]-5-[(2-methyl-6-quinolinyl)methylidene]-1,3-thiazol-4(5H)-one;
(5Z)-5-[(7-Chloro-6-quinolinyl)methylidene]-2-[(2,6-dichlorophenyl)amino]-1,3-thiazol-4(5H)-one trifluoroacetate;
(5Z)-5-[(8-Chloro-6-quinolinyl)methylidene]-2-[(2,6-dichlorophenyl)amino]-1,3-thiazol-4(5H)-one, trifluoroacetate salt;
(5Z)-2-[(2-Chlorophenyl)amino]-5-[(8-methyl-6-quinolinyl)methylidene]-1,3-thiazol-4(5H)-one;
(5Z)-5-[(5-Chloro-6-quinolinyl)methylidene]-2-[(2,6-dichlorophenyl)amino]-1,3-thiazol-4(5H)-one;
(5Z)-2-[(2,6-Dichlorophenyl)amino]-5-[(2-pentyl-6-quinolinyl)methylidene]-1,3-thiazol-4(5H)-one;
6-{(Z)-[2-[(2,6-Dichlorophenyl)amino]-4-oxo-1,3-thiazol-5(4H)-ylidene]methyl}-2(1 H)-quinolinone;
(5Z)-2-[(2,6-Dichlorophenyl)amino]-5-[(2-ethyl-6-quinolinyl)methylidene]-1,3-thiazol-4(5H)-one;
(5Z)-2-[(2,6-Dichlorophenyl)amino]-5-{[2-(methyloxy)-6-quinolinyl]methylidene}-1,3-thiazol-4(5H)-one;
(5Z)-2-[(2,6-Dichlorophenyl)amino]-5-{[2-(dimethylamino)-6-quinolinyl]methylidene}-1,3-thiazol-4(5H)-one;
(5Z)-2-[(2,6-Dichlorophenyl)amino]-5-[(4-hydroxy-6-quinolinyl)methylidene]-1,3-thiazol-4(5H)-one;
(5Z)-2-[(2,6-Dichlorophenyl)amino]-5-[(3-methyl-6-quinolinyl)methylidene]-1,3-thiazol-4(5H)-one;
(5Z)-2-[(2,6-Dichlorophenyl)amino]-5-{[3-(1-methylethyl)-6-quinolinyl]methylidene}-1,3-thiazol-4(5H)-one;
(5Z)-2-[(2,6-Dichlorophenyl)amino]-5-{[4-(methylamino)-6-quinolinyl]methylidene}-1,3-thiazol-4(5H)-one;
Ethyl-4-chloro-6-{(Z)-[2-[(2,6-dichlorophenyl)amino]-4-oxo-1,3-thiazol-5(4H)-ylidene]methyl}-3-quinolinecarboxylate;
(5Z)-5-[(4-Chloro-6-quinolinyl)methylidene]-2-[(2,6-dichlorophenyl)amino]-1,3-thiazol-4(5H)-one;
(5Z)-2-[(2,6-Dichlorophenyl)amino]-5-{[4-(methyloxy)-6-quinolinyl]methylidene}-1,3-thiazol-4(5H)-one;
(5Z)-2-[(2,6-Dichlorophenyl)amino]-5-{[4-(1-piperidinyl)-6-quinolinyl]methylidene}-1,3-thiazol-4(5H)-one trifluoroacetate;
(5Z)-2-[(2,6-Dichlorophenyl)amino]-5-{[4-(4-morpholinyl)-6-quinolinyl]methylidene}-1,3-thiazol-4(5H)-one;
(5Z)-2-[(2,6-Dichlorophenyl)amino]-5-[(8-fluoro-6-quinolinyl)methylidene]-1,3-thiazol-4(5H)-one;
(5Z)-2-[(2,6-Dichlorophenyl)amino]-5-({4-[(3,3-dimethylbutyl)amino]-6-quinolinyl}methylidene)-1,3-thiazol-4(5H)-one trifluoroacetate;
Ethyl-6-{(Z)-[2-[(2,6-dichlorophenyl)amino]-4-oxo-1,3-thiazol-5(4H)-ylidene]methyl}-3-quinolinecarboxylate;
6-{(Z)-[2-[(2,6-Dichlorophenyl)amino]-4-oxo-1,3-thiazol-5(4H)-ylidene]methyl}-3-quinolinecarboxylic acid;
6-{(Z)-[2-[(2,6-Dichlorophenyl)amino]-4-oxo-1,3-thiazol-5(4H)-ylidene]methyl}-3-quinolinecarboxamide;
(5Z)-5-({4-[(2-Cyclopropylethyl)amino]-6-quinolinyl}methylidene)-2-[(2,6-dichlorophenyl)amino]-1,3-thiazol-4(5H)-one trifluoroacetate;
(5Z)-2-[(2,6-Dichlorophenyl)amino]-5-{[4-(1-pyrrolidinyl)-6-quinolinyl]methylidene}-1,3-thiazol-4(5H)-one;
(5Z)-2-[(2,6-Dichlorophenyl)amino]-5-{[3-(hydroxymethyl)-6-quinolinyl]methylidene}-1,3-thiazol-4(5H)-one;
6-{(Z)-[2-[(2,6-Dichlorophenyl)amino]-4-oxo-1,3-thiazol-5(4H)-ylidene]methyl}-N-methyl-3-quinolinecarboxamide hydrochloride;
6-{(Z)-[2-[(2,6-Dichlorophenyl)amino]-4-oxo-1,3-thiazol-5(4H)-ylidene]methyl}-N,N-dimethyl-3-quinolinecarboxamide;
(5Z)-2-[(2,6-Dichlorophenyl)amino]-5-[(4-phenyl-6-quinolinyl)methylidene]-1,3-thiazol-4(5H)-one trifluoroacetate;
(5Z)-2-[(3-Chloro-2-biphenylyl)amino]-5-[(4-phenyl-6-quinolinyl)methylidene]-1,3-thiazol-4(5H)-one trifluoroacetate;
(5Z)-2-[(2,6-Dichlorophenyl)amino]-5-[(4-methyl-6-quinolinyl)methylidene]-1,3-thiazol-4(5H)-one;
(5Z)-2-[(2,6-Dichlorophenyl)amino]-5-{[4-(1-methylethyl)-6-quinolinyl]methylidene}-1,3-thiazol-4(5H)-one;
(5Z)-2-[(2,6-Dichlorophenyl)amino]-5-[(4-ethyl-6-quinolinyl)methylidene]-1,3-thiazol-4(5H)-one;
6-{(Z)-[2-[(2,6-Dichlorophenyl)amino]-4-oxo-1,3-thiazol-5(4H)-ylidene]methyl}-4-quinolinecarbonitrile;
(5Z)-2-[(2,6-Dichlorophenyl)amino]-5-{[4-(4-pyridinyl)-6-quinolinyl]methylidene}-1,3-thiazol-4(5H)-one;
(5Z)-2-[(2,6-Dichlorophenyl)amino]-5-{[4-(3-pyridinyl)-6-quinolinyl]methylidene}-1,3-thiazol-4(5H)-one;
Ethyl-6-{(Z)-[2-[(2,6-dichlorophenyl)amino]-4-oxo-1,3-thiazol-5(4H)-ylidene]methyl}-4-(4-morpholinyl)-3-quinolinecarboxylate;
Ethyl-6-{(Z)-[2-[(2,6-dichlorophenyl)amino]-4-oxo-1,3-thiazol-5(4H)-ylidene]methyl}-4-(4-methyl-1-piperazinyl)-3-quinolinecarboxylate;
6-{(Z)-[2-[(2,6-Dichlorophenyl)amino]-4-oxo-1,3-thiazol-5(4H)-ylidene]methyl}-4-(4-morpholinyl)-3-quinolinecarboxamide trifluoroacetate;
N-{4-Chloro-3-[((5Z)-5-{[4-(4-morpholinyl)-6-quinolinyl]methylidene}-4-oxo-4,5-dihydro-1,3-thiazol-2-yl)amino]phenyl}cyclobutanecarboxamide;
N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}cyclobutanecarboxamide;
6-{(Z)-[2-[(2,6-Dichlorophenyl)amino]-4-oxo-1,3-thiazol-5(4H)-ylidene]methyl}-4-(4-methyl-1-piperazinyl)-3-quinolinecarboxamide trifluoroacetate;
(5Z)-2-[(2,6-Dichlorophenyl)amino]-5-{[4-(1-piperazinyl)-6-quinolinyl]methylidene}-1,3-thiazol-4(5H)-one;
N-{4-Chloro-3-[((5Z)-4-oxo-5-{[4-(1-piperazinyl)-6-quinolinyl]methylidene}-4,5-dihydro-1,3-thiazol-2-yl)amino]phenyl}cyclobutanecarboxamide;
Ethyl-6-{(Z)-[2-[(2,6-dichlorophenyl)amino]-4-oxo-1,3-thiazol-5(4H)-ylidene]methyl}-4-(dimethylamino)-3-quinolinecarboxylate;
Ethyl-6-{(Z)-[2-[(2,6-dichlorophenyl)amino]-4-oxo-1,3-thiazol-5(4H)-ylidene]methyl}-4-(methylamino)-3-quinolinecarboxylate;
Ethyl-6-{(Z)-[2-({2-chloro-5-[(cyclobutylcarbonyl)amino]phenyl}amino)-4-oxo-1,3-thiazol-5(4H)-ylidene]methyl}-4-(dimethylamino)-3-quinolinecarboxylate trifluoroacetate;
Ethyl-6-{(Z)-[2-({2-chloro-5-[(cyclobutylcarbonyl)amino]phenyl}amino)-4-oxo-1,3-thiazol-5(4H)-ylidene]methyl}-4-(methylamino)-3-quinolinecarboxylate;
Ethyl-6-{(Z)-[2-({2-chloro-5-[(cyclobutylcarbonyl)amino]phenyl}amino)-4-oxo-1,3-thiazol-5(4H)-ylidene]methyl}-4-(4-morpholinyl)-3-quinolinecarboxylate trifluoroacetate;
Ethyl-6-{(Z)-[2-[(2,6-dichlorophenyl)amino]-4-oxo-1,3-thiazol-5(4H)-ylidene]methyl}-4-(4-pyridinyl)-3-quinolinecarboxylate trifluoroacetate;
Ethyl-6-{(Z)-[2-({2-chloro-5-[(cyclobutylcarbonyl)amino]phenyl}amino)-4-oxo-1,3-thiazol-5(4H)-ylidene]methyl}-4-(4-pyridinyl)-3-quinolinecarboxylate trifluoroacetate;
6-{(Z)-[2-[(2,6-Dichlorophenyl)amino]-4-oxo-1,3-thiazol-5(4H)-ylidene]methyl}-4-(4-pyridinyl)-3-quinolinecarboxamide trifluoroacetate;
6-{(Z)-[2-[(2,6-Dichlorophenyl)amino]-4-oxo-1,3-thiazol-5(4H)-ylidene]methyl}-4-quinolinecarboxylic acid trifluoroacetate;
6-{(Z)-[2-[(2,6-Dichlorophenyl)amino]-4-oxo-1,3-thiazol-5(4H)-ylidene]methyl}-4-quinolinecarboxamide trifluoroacetate;
Methyl-6-{(Z)-[2-[(2,6-dichlorophenyl)amino]-4-oxo-1,3-thiazol-5(4H)-ylidene]methyl}-4-quinolinecarboxylate trifluoroacetate;
6-{(Z)-[2-[(2,6-Dichlorophenyl)amino]-4-oxo-1,3-thiazol-5(4H)-ylidene]methyl}-N-methyl-4-quinolinecarboxamide trifluoroacetate;
6-{(Z)-[2-[(2,6-Dichlorophenyl)amino]-4-oxo-1,3-thiazol-5(4H)-ylidene]methyl}-N,N-dimethyl-4-quinolinecarboxamide trifluoroacetate;
(5Z)-2-[(2,6-Dichlorophenyl)amino]-5-{[4-(4-morpholinylcarbonyl)-6-quinolinyl]methylidene}-1,3-thiazol-4(5H)-one trifluoroacetate;
(5Z)-2-[(2,6-Dichlorophenyl)amino]-5-({4-[(4-methyl-1-piperazinyl)carbonyl]-6-quinolinyl}methylidene)-1,3-thiazol-4(5H)-one trifluoroacetate;
(5Z)-2-[(2,6-Dichlorophenyl)amino]-5-{[3-(dimethylamino)-6-quinoxalinyl]methylidene}-1,3-thiazol-4(5H)-one;
(5Z)-2-[(2,6-Dichlorophenyl)amino]-5-({3-[2-(dimethylamino)ethyl]-6-quinoxalinyl}methylidene)-1,3-thiazol-4(5H)-one;
Ethyl-6-{(Z)-[2-({2-chloro-5-[(2-methylpropanoyl)amino]phenyl}amino)-4-oxo-1,3-thiazol-5(4H)-ylidene]methyl}-4-(4-pyridinyl)-3-quinolinecarboxylate;
(5Z)-2-[(2,6-Dichlorophenyl)amino]-5-{[3-(methyloxy)-6-quinoxalinyl]methylidene}-1,3-thiazol-4(5H)-one;
(5Z)-2-[(2,6-Dichlorophenyl)amino]-5-{[3-(4-methyl-1-piperazinyl)-6-quinoxalinyl]methylidene}-1,3-thiazol-4(5H)-one;
(5Z)-2-[(2,6-Dichlorophenyl)amino]-5-[(3-methyl-6-quinoxalinyl)methylidene]-1,3-thiazol-4(5H)-one;
(5Z)-2-[(2,6-Dichlorophenyl)amino]-5-[(3-phenyl-6-quinoxalinyl)methylidene]-1,3-thiazol-4(5H)-one;
(5Z)-2-[(2,6-Dichlorophenyl)amino]-5-({3-[(2-hydroxyethyl)(methyl)amino]-6-quinoxalinyl}methylidene)-1,3-thiazol-4(5H)-one;
(5Z)-2-[(2,6-Dichlorophenyl)amino]-5-({3-[[2-(dimethylamino)ethyl](methyl)amino]-6-quinoxalinyl}methylidene)-1,3-thiazol-4(5H)-one;
(5Z)-2-[(2,6-Dichlorophenyl)amino]-5-{[3-(phenylamino)-6-quinoxalinyl]methylidene}-1,3-thiazol-4(5H)-one;
N-{4-Chloro-3-[((5Z)-5-{[3-(4-morpholinyl)-6-quinoxalinyl]methylidene}-4-oxo-4,5-dihydro-1,3-thiazol-2-yl)amino]phenyl}cyclobutanecarboxamide;
(5Z)-5-[(3-Amino-6-quinoxalinyl)methylidene]-2-[(2,6-dichlorophenyl)amino]-1,3-thiazol-4(5H)-one;
N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}-2-methylpropanamide trifluoroacetate;
N-{4-Chloro-3-[((5Z)-5-{[4-(4-morpholinyl)-6-quinolinyl]methylidene}-4-oxo-4,5-dihydro-1,3-thiazol-2-yl)amino]phenyl}-2-methylpropanamide trifluoroacetate;
N-{4-Chloro-3-[((5Z)-5-{[4-(4-methyl-1-piperazinyl)-6-quinolinyl]methylidene}-4-oxo-4,5-dihydro-1,3-thiazol-2-yl)amino]phenyl}ethanesulfonamide trifluoroacetate;
N-{4-Chloro-3-[((5Z)-5-{[4-(4-morpholinyl)-6-quinolinyl]methylidene}-4-oxo-4,5-dihydro-1,3-thiazol-2-yl)amino]phenyl}ethanesulfonamide trifluoroacetate;
Ethyl-6-{(Z)-[2-({2-chloro-5-[(2-methylpropanoyl)amino]phenyl}amino)-4-oxo-1,3-thiazol-5(4H)-ylidene]methyl}-4-(4-morpholinyl)-3-quinolinecarboxylate trifluoroacetate;
Ethyl-6-{(Z)-[2-({2-chloro-5-[(2-methylpropanoyl)amino]phenyl}amino)-4-oxo-1,3-thiazol-5(4H)-ylidene]methyl}-3-quinolinecarboxylate trifluoroacetate;
Ethyl-6-{(Z)-[2-({2-chloro-5-[(2-methylpropanoyl)amino]phenyl}amino)-4-oxo-1,3-thiazol-5(4H)-ylidene]methyl}-4-(4-methyl-1-piperazinyl)-3-quinolinecarboxylate trifluoroacetate; and
Ethyl-6-{(Z)-[2-({2-chloro-5-[(cyclobutylcarbonyl)amino]phenyl}amino)-4-oxo-1,3-thiazol-5(4H)-ylidene]methyl}-3-quinolinecarboxylate trifluoroacetate.

10. A pharmaceutically acceptable salt, hydrate, solvate or pro-drug of a compound of claim 9.

11. A pharmaceutical composition comprising a compound according to claim 1, and/or a pharmaceutically acceptable salt, hydrate, solvate or pro-drug thereof and a pharmaceutically acceptable carrier.

12. A process for preparing a pharmaceutical composition containing a pharmaceutically acceptable carrier or diluent and an effective amount of a compound of Formula (I) as described in claim 1 and/or a pharmaceutically acceptable salt, hydrate, solvate or pro-drug thereof, which process comprises bringing the compound of Formula (I) and/or a pharmaceutically acceptable salt, hydrate, solvate or pro-drug thereof into association with a pharmaceutically acceptable carrier or diluent.

13. A method of inhibiting hYAK3 in a mammal; comprising, administering to the mammal a therapeutically effective amount of a compound of the Formula I, as described in claim 1, or a pharmaceutically acceptable salt, hydrate, solvate or pro-drug thereof.

14. A method of treating or preventing diseases of the erythroid and hematopoietic systems, caused by hYAK3 activity; comprising, administering to a mammal a therapeutically effective amount of a compound of claim 1, or a pharmaceutically acceptable salt, hydrate, solvate or pro-drug thereof and one or more of pharmaceutically acceptable carriers, diluents and excipients.

15. A method of claim 14 in which diseases of the erythroid and hematopoietic systems are selected from the group consisting of: anemia, aplastic anemia, myelodysplastic syndrome, myelosuppression, and cytopenia.

16. A method of treating or preventing diseases selected from the group consisting of: anemia, aplastic anemia, myelodysplastic syndrome, myelosuppression, and cytopenia; comprising, administering to a mammal a therapeutically effective amount of a compound of claim 1, or a pharmaceutically acceptable salt, hydrate, solvate or pro-drug thereof and one or more of pharmaceutically acceptable carriers, diluents and excipients.

17. The method of claim 14 wherein the mammal is a human.

18. A method of treating diseases of the hematopoietic system, in a mammal in need thereof, which comprises: administering to such mammal a therapeutically effective amount of
a) a compound of Formula (I), as described in claim 1 and/or a pharmaceutically acceptable salt, hydrate, solvate or pro-drug thereof; and
b) EPO or a derivative thereof.
